(19) United States
(12) Patent Application Publication
(10) Pub. No.: US 2003/0064426 A1
(43) Pub. Date: Apr. 3, 2003

(54) Reagents and methods for identifying and modulating expression of genes regulated by CDK inhibitors

(76) Inventors: Jason Poole, Chicago, IL (US); Bey-Dih Chang, Lombard, IL (US); Igor B. Roninson, Wilmette, IL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/861,925

(22) Filed: May 21, 2001

Related U.S. Application Data
(60) Provisional application No. 60/265,840, filed on Feb. 1, 2001.

(51) Int. Cl. C12Q 1/66; C12N 9/99; C12P 21/02; C12N 5/06

(52) U.S. Cl. 435/8; 435/184; 435/320.1; 435/325; 435/69.1

(57) ABSTRACT

This invention provides methods and reagents for identifying compounds that inhibit the induction of genes involved in cancer and age-related diseases, such genes being induced by cyclin-dependent kinase inhibitors.

This application claims priority to U.S. Provisional Application Ser. No. 60/______, filed Feb. 1, 2001.

This application was supported by a grant from the National Institutes of Health, No. R01CA62099. The government may have certain rights in this invention.

### BACKGROUND OF THE INVENTION

1. Field of the Invention

This invention is related to cellular senescence and changes in cellular gene expression that accompany senescence. In particular, the invention is related to the identification of genes the expression of which is modulated by a class of cellular gene products termed cyclin dependent kinase (CDK) inhibitors, induced in cells at the onset of senescence. More specifically, the invention provides markers of cellular senescence that are genes whose expression in induced by such CDK inhibitors. The invention provides methods for identifying compounds that inhibit pathological consequences of cellular senescence by detecting inhibition of induction of these marker genes by CDK inhibitors in the presence of such compounds. Also provided are reagents that are recombinant mammalian cells containing recombinant expression constructs encoding different cellular CDK inhibitors, such as p21 and p16 that are experimentally-inducible, and recombinant mammalian cells containing a recombinant expression construct that expresses a reporter gene under the transcriptional control of a promoter for a gene whose expression is induced by endogenous or exogenous, experimentally-inducible, CDK inhibitors.

2. Summary of the Related Art

Cell cycle progression is regulated to a large extent by a set of serine/threonine kinases, known as cyclin-dependent kinases (CDKs). A special group of proteins, known as CDK inhibitors, interact with and inhibit CDKs, thus causing cell cycle arrest in a variety of physiological situations (see Sielecki et al., 2000, J Med. Chem. 43: 1-18 and references therein). There are two families of CDK inhibitors. The first one, known as Cip/Kip, includes p21^{Waf1/Cip1/Sdi1}, p27^{Kip1}, and p57^{Kip2}. The second family, Ink4, includes p16^{Ink4A}, p15^{Ink4b}, p18^{Ink4c}, and p19^{Ink4d}. Expression of specific CDK inhibitors is activated by different factors. For example, contact inhibition induces p27 and p16 expression (Dietrich et al., 1997, Oncogene 15: 2743-2747), extracellular anti-mitogenic factors such as TGFα induce p15 expression (Reynisdottir et al., 1995, Genes Dev. 9: 1831-1845), serum starvation induces p27 expression (Polyak et al., 1994, Genes Dev. 8: 9-22), and UV radiation induces p16 expression (Wang et al., 1996, Cancer Res. 56: 2510-2514). In addition, all of the above treatments, as well as different forms of DNA damage induce expression of p21, the most pleiotropic of the known CDK inhibitors (Dotto, 2000, BBA Rev. Cancer 1471: M43-M56).

Of special importance to the field of this invention, two of the CDK inhibitors, p21 and p16, have been intimately associated with the process of senescence in mammalian cells. At the onset of replicative senescence (Alcorta et al., 1996, Proc. Natl. Acad. Sci. USA 93:13742-13747) and damage-induced accelerated senescence (Robles & Adami, 1998, Oncogene 16: 1113-1123), p21 induction results in cell growth arrest. This surge of p21 expression is transient, however, and is followed by stable activation of p16, which is believed to be responsible for the maintenance of growth arrest in senescent cells. The knockout of p21 (Brown et al., 1997, Science 277: 831-834) or p16 (Serrano et al., 1996, Cell 85: 27-37) delays or prevents the onset of senescence. Furthermore, ectopic overexpression of either p21 or p16 induces growth arrest accompanied by phenotypic markers of senescence in both normal and tumor cells (Vogt et al., 1998, Cell Growth Differ. 9: 139-146; McConnell et al., 1998, Curr. Biol. 8: 351-354; Fang et al., 1999, Oncogene 18: 2789-2797).

p21 has been independently identified by several groups as a protein that binds and inhibits CDKs (Harper et al., 1993, Cell 75: 805-816), as a gene upregulated by wild-type p53 (el-Deiry et al., 1993, Cancer Res. 55: 2910-2919), and as a growth-inhibitory gene overexpressed in senescent fibroblasts (Noda et al., 1994, Exp. Cell. Res. 211: 90-98). Because of its pivotal role in p53-regulated growth arrest, p21 is usually regarded as a tumor suppressor. Nevertheless, p21 mutations in human cancer are rare (Hall & Peters, 1996, Adv. Cancer Res. 68: 67-108), and p21 knockout mice develop normally and do not show an increased rate of tumorigenesis (Deng et al., 1995, Cell 82: 675-684).

Cellular levels of p21 are increased in response to a variety of stimuli, including DNA-damaging and differentiating agents. Some of these responses are mediated through transcriptional activation of the p21 gene by p53, but p21 is also regulated by a variety of p53-independent factors (reviewed in Gartel & Tyner, 1999, Exp. Cell Res. 227: 171-181).

Transient induction of p21 mediates different forms of damage-induced growth arrest, including transient arrest that allows cells to repair DNA damage, as well as permanent growth arrest (also termed “accelerated senescence”), which is induced in normal fibroblasts (DiLeonardo et al., 1994, Genes Develop. 8: 2540-2551; Robles & Adami, 1998, Oncogene 16: 1113-1123) and tumor cells (Chang et al., 1999, Cancer Res. 59: 3761-3767) by DNA damage or introduction of oncogenic RAS (Serrano et al., 1997, Cell 88: 593-602). A surge of p21 expression also coincides with the onset of terminal growth arrest during replicative senescence of aging fibroblasts (Noda et al., 1994, ibid.; Alcorta et al., 1996, Proc. Natl. Acad. Sci USA 93:13742-13747; Stein et al., 1999, Mol Cell. Biol. 19: 2109-2117) and terminal differentiation of postmitotic cells (E1-Deiry et al., 1995, ibid.; Gartel et al, 1996, Exp. Cell Res. 246: 280-289).

While p21 is not a transcription factor per se, it has indirect effects on cellular gene expression that may play a role in its cellular functions (Dotto, 2000, BBA Rev. Cancer 1471:M43-M56 and references therein). One of the consequences of CDK inhibition by p21 is dephosphorylation of Rb, which in turn inhibits E2F transcription factors that regulate many genes involved in DNA replication and cell cycle progression (Nevins, 1998, Cell Growth Differ. 9: 585-593). A comparison of p21-expressing cells (p21 +/+) and p21 -nonexpressing cells (p21 −/−) has implicated p21 in radiation-induced inhibition of several genes involved in cell cycle progression (de Toledo et al., 1998, Cell Growth Differ. 9: 887-896). Another result of CDK inhibition by p21 is stimulation of the transcription cofactor histone acetyltransferase p300, that enhances many inducible transcription factors including NFκB (Perkins et al., 1988, Science 275: 523-527). Activation of p300 may have a pleiotropic effect on gene expression (Snowden & Perkins, 1988, Biochem. Pharmacol. 55: 1947-1954). p21 may also affect gene expression through its interactions with many transcriptional regulators and coregulators other than CDK, such as JNK kinases, apoptosis signal-regulating kinase 1, Myc and others (Dotto, 2000, BBA Rev. Cancer 1471 :M43-M56). These interactions may affect the expression of genes regulated by the corresponding pathways.

Another CDK inhibitor of particular relevance to the present invention is p16^{INK4A}; the human protein has been described by Serrano et al. (1993, Nature 366:

704-707). As mentioned above, p16 is an essential regulator of senescence in mammalian cells. It is also a bonafide tumor suppressor and one of the most commonly mutated genes in human cancers (Hall & Peters, 1996, Adv. Cancer Res. 68: 67-108). p16 is known to directly inhibit CDK4 and CDK6, and may indirectly inhibit CDK2 as well (McConnell et al., 1999, Molec. Cell. Biol. 19: 1981-1989).

There remains a need in this art to identify genes whose expression is modulated by induction of CDK inhibitor genes such as p21 and p16. There is also a need in this art to develop targets for assessing the effects of compounds on cellular senescence, carcinogenesis and age-related diseases.

### SUMMARY OF THE INVENTION

This invention provides reagents and methods for identifying genes whose expression is modulated by induction of CDK inhibitor gene expression. The invention also provides reagents and methods for identifying compounds that inhibit the effects of CDK inhibitors such as p21 and p16 on cellular gene expression, as a first step in rational drug design for preventing pathogenic consequences of cellular senescence, such as carcinogenesis and age-related diseases.

In a first aspect, the invention provides a mammalian cell containing an inducible CDK inhibitor gene. In preferred embodiments, the CDK inhibitor gene encodes p21 or p16. In preferred embodiments, the mammalian cell is a recombinant mammalian cell comprising a recombinant expression construct encoding an inducible p21 gene or an inducible p16 gene. More preferably, the construct comprises a nucleotide sequence encoding p21, most preferably human p21, under the transcriptional control of an inducible promoter. In alternative embodiments, the construct comprises a nucleotide sequence encoding the amino-terminal portion of p21 comprising the CDK binding domain, more preferably comprising amino acids 1 through 78 of the p21 amino acid sequence. In additional embodiments, the construct comprises a nucleotide sequence encoding p16, most preferably human p16, under the transcriptional control of an inducible promoter. In preferred embodiments, the inducible promoter in each such construct can be induced by contacting the cells with an inducing agent, most preferably a physiologically-neutral inducing agent, that induces transcription from the inducible promoter or by removing an agent that inhibits transcription from such promoter. Preferred cells include mammalian cells, preferably rodent or primate cells, and more preferably mouse or human cells. In a particularly preferred embodiment are fibrosarcoma cells, more preferably human fibrosarcoma cells and most preferably human HT1080 fibrosarcoma cell line and derivatives thereof.

In another embodiment of the first aspect of the invention are provided recombinant mammalian cells comprising a recombinant expression construct in which a reporter gene is under the transcriptional control of a promoter derived from a cellular gene whose expression is modulated by a CDK inhibitor, most preferably p21 or p 16. In a preferred embodiment, the promoter is derived from a cellular gene whose expression induced by a CDK inhibitor such as p21 or p 16. In these embodiments, the promoter is most preferably derived from a gene identified in Table II; however, those with skill in the art will recognize that a promoter from any gene whose expression is induced by CDK inhibitor gene expression can be advantageously used in such constructs. Most preferably, the promoter is derived from serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p₆₆^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), prostacyclin stimulating factor (SEQ ID NO.: 18), vascular endothelial growth factor-C (SEQ ID NO.19) and tissue inhibitor of metalloproteinases-1 (SEQ ID NO. 20). Preferred reporter genes comprising the recombinant expression constructs of the invention include firefly luciferase, Renilla luciferase, chloramphenicol acetyltransferase, beta-galactosidase, green fluorescent protein, or alkaline phosphatase.

In additional preferred embodiments, the invention provides a mammalian cell comprising a first recombinant expression construct encoding a reporter gene under the transcriptional control of a promoter for a mammalian gene whose expression is modulated by a CDK inhibitor, most preferably p21 or p16, and a second recombinant expression construct encoding a mammalian CDK inhibitor gene, wherein expression of the CDK inhibitor is experimentally-induced in the mammalian cell thereby. In preferred embodiments, the CDK inhibitor gene is p16 or p21. In preferred embodiments, the recombinant expression construct encoding a mammalian CDK inhibitor gene is under the transcriptional control of an inducible heterologous promoter, wherein expression of the CDK inhibitor from the recombinant expression construct is mediated by contacting the recombinant cell with an inducing agent that induces transcription from the inducible promoter or by removing an agent that inhibits transcription from such promoter. Preferably, the construct comprises a nucleotide sequence encoding p21, most preferably human p21. In other embodiments, the construct comprises a nucleotide sequence encoding the amino-terminal portion of p21 comprising the CDK binding domain, more preferably comprising amino acids 1 through 78 of the p21 amino acid sequence. In alternative preferred embodiments, the construct comprises a nucleotide sequence encoding p 16, most preferably human p16. In a preferred embodiment of the second recombinant expression construct encoding a reporter gene, the promoter is derived from a cellular gene whose expression is induced by a CDK inhibitor such as p21 or p16. In these embodiments, the promoter is most preferably derived from a gene identified in Table II. Preferred reporter genes comprising the second recombinant expression constructs of the invention include firefly luciferase, Renilla luciferase, chloramphenicol acetyltransferase, beta-galactosidase, green fluorescent protein, or alkaline phosphatase. In a particularly preferred embodiment are fibrosarcoma cells, more preferably human fibrosarcoma cells and most preferably human HT1080 fibrosarcoma cell line and derivatives thereof. The product of the reporter gene or an endogenous gene that is induced by the CDK inhibitor is preferably detected using an immunological reagent, by assaying for an activity of the gene product, or by hybridization to a complementary nucleic acid.

In a second aspect, provides a screening method for identifying compounds that inhibit CDK inhibitor-induced expression of mitogenic or anti-apoptotic factors in mammalian cells. In preferred embodiments, the method comprises the steps of inducing the expression of a CDK inhibitor, most preferably p21 or p16, in the cells in the presence or absence of a compound, and comparing expression of a mitogen or anti-apoptotic compound, or a plurality thereof, in the conditioned media. Inhibitors of CDK inhibitor effects are identified by having a lesser amount of the mitogen or anti-apoptotic compound, or a plurality thereof, in the conditioned media in the presence of the compound than in the absence of the compound. In the methods provided in this aspect of the invention, any CDK inhibitor-expressing cell is useful, most preferably cells expressing p21 or p16, and p21 or p16 expression in such cells can be achieved by inducing endogenous p21 or p16, or by using cells containing an inducible expression construct encoding p21 or p16 according to the invention. Preferred cells include mammalian cells, preferably rodent or primate cells, and more preferably mouse or human cells. In a particularly preferred embodiment are fibrosarcoma cells, more preferably human fibrosarcoma cells and most preferably human HT1080 fibrosarcoma cell line and derivatives thereof. Mitogen or anti-apoptosis compound expression is detected using an immunological reagent, by assaying for an activity of the gene product, or by hybridization to a complementary nucleic acid.

In alternative embodiments, the invention provides methods for identifying compounds that inhibit CDK inhibitor-induced expression of mitogenic or anti-apoptotic factors in mammalian cells, wherein the cells comprise a recombinant expression construct encoding a reporter gene under the transcriptional control of a promoter of a cellular gene encoding a mitogenic or anti-apoptotic factor that is induced by a CDK inhibitor such as p21 or p16. In preferred embodiments, promoters include the promoters for connective tissue growth factor (CTGF; SEQ ID NO. 3), activin A (SEQ ID NO. 5), epithelin/granulin (SEQ ID NO. 11), galectin-3 (SEQ ID NO. 9), prosaposin (SEQ ID NO. 7), clusterin (SEQ ID NO. 17), prostacyclin stimulating factor (SEQ ID NO.: 18), vascular endothelial growth factor-C (SEQ ID NO.: 19) and tissue inhibitor of metalloproteinases (SEQ ID NO.: 20). Preferred reporter genes include but are not limited to firefly luciferase, Renilla luciferase, β-galactosidase, alkaline phosphatase and green fluorescent protein. In these embodiments, inhibition of CDK inhibitor-mediated induction of reporter gene expression is used to identify compounds that inhibit induction of mitogens or anti-apoptotic factors in CDK inhibitor-expressing cells.

In this aspect, the invention also provides a method for inhibiting production of mitogenic or anti-apoptotic factors or compounds in a mammalian cell, the method comprising the steps of contacting the cell with a compound that inhibits production of mitogenic or anti-apoptotic factors, wherein said compound is identified by the aforesaid methods of this aspect of the invention. In preferred embodiments, the mammalian cells contacted with the inhibitory compounds in which production of mitogenic or anti-apoptotic factors is inhibited are fibroblasts, most preferably stromal fibroblasts. In preferred embodiments, the compounds are inhibitors of nuclear factor kappa-B (NFκB) activity or expression.

In a third aspect, the invention provides methods for identifying compounds that inhibit CDK inhibitor-mediated induction of cellular gene expression. These methods comprise the steps of inducing or otherwise producing expression of a CDK inhibitor gene in a mammalian cell; assaying the cell in the presence of the compound for changes in expression of cellular genes whose expression is induced by the CDK inhibitor; and identifying compounds that inhibit CDK inhibitor-mediated induction of cellular gene expression if expression of the cellular genes is changed to a lesser extent in the presence of the compound than in the absence of the compound. In preferred embodiments, the CDK inhibitor is p21 or p16. In preferred embodiments, the cellular genes are induced by a CDK inhibitor, and compounds that inhibit this induction of cellular gene expression are detected by detecting expression of the genes at levels less than those detected when the CDK inhibitor is expressed in the absence of the compound. In preferred embodiments of this aspect of the inventive methods, the CDK inhibitor is p21 or p16. In preferred embodiments, the genes are identified in Table II. In further alternative embodiments, the method is performed using a recombinant mammalian cell comprising a reporter gene under the transcriptional control of a promoter derived from a gene whose expression is induced by a CDK inhibitor. When using constructs comprising promoters derived from genes induced by a CDK inhibitor, the reporter gene product is produced at lesser levels in the presence than the absence of the compound when the compound inhibits or otherwise interferes with CDK inhibitor-mediated gene expression modulation. In preferred embodiments of this aspect of the inventive methods, the CDK inhibitor is p21 or p16. In these embodiments, the promoter is most preferably derived from a gene identified in Table II. Most preferably, the promoter is derived from serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), Prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p66^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), prostacyclin stimulating factor (SEQ ID NO.: 18), vascular endothelial growth factor-C (SEQ ID NO.19) and tissue inhibitor of metalloproteinases-1 (SEQ ID NO. 20). Preferred reporter genes comprising the recombinant expression constructs of the invention include firefly luciferase, Renilla luciferase, chloramphenicol acetyltransferase, beta-galactosidase, green fluorescent protein, or alkaline phosphatase. In other preferred embodiments, the cell comprises a first recombinant expression construct encoding a reporter gene under the transcriptional control of a promoter for a mammalian gene whose expression is induced by a CDK inhibitor, and a second recombinant expression construct encoding a mammalian CDK inhibitor gene, wherein expression of the CDK inhibitor is experimentally-induced in the mammalian cell thereby. The product of the reporter gene or the endogenous gene that is induced by the CDK inhibitor is preferably detected using an immunological reagent, by assaying for an activity of the gene product, or by hybridization to a complementary nucleic acid.

In a fourth aspect, the invention provides methods for identifying compounds that inhibit pathogenic consequences of senescence in a mammalian cell, wherein such pathogenic consequences are mediated at least in part by expression of genes induced by CDK inhibitors. These methods comprise the steps of treating the mammalian cell in the presence of the compound with an agent or culturing the mammalian cell under conditions that induce CDK inhibitor gene expression; assaying the mammalian cell for induction of genes that are induced by CDK inhibitors; and identifying the compound as an inhibitor of senescence or pathogenic consequences of senescence if expression of genes that are induced by the CDK inhibitor are induced to a lesser extent in the presence of the compound than in the absence of the compound. In preferred embodiments of this aspect of the inventive methods, the CDK inhibitor is p21 or p16. In preferred embodiments, the genes are identified in Table II. In further alternative embodiments, the method is performed using a recombinant mammalian cell comprising a reporter gene under the transcriptional control of a promoter derived from a gene whose expression is modulated by a CDK inhibitor. In these embodiments, production of the product of the reporter gene at lesser levels in the presence than the absence of the compound using constructs comprising promoter derived from genes induced by the CDK inhibitor, is detected when the compound is an inhibitor of pathogenic consequences of cell senescence. In preferred embodiments of this aspect of the inventive methods, the CDK inhibitor is p21 or p16. The promoters are preferably derived from genes identified in Table II. The promoter most preferably is derived from serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p66^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), prostacyclin stimulating factor (SEQ ID NO.: 18), vascular endothelial growth factor-C (SEQ ID NO. 19) and tissue inhibitor of metalloproteinases-1 (SEQ ID NO. 20). In other preferred embodiments, the cell comprises a first recombinant expression construct encoding a reporter gene under the transcriptional control of a promoter for a mammalian gene whose expression is induced by a CDK inhibitor, and a second recombinant expression construct encoding a mammalian CDK inhibitor gene, wherein expression of the CDK inhibitor is experimentally-induced in the mammalian cell thereby. In preferred embodiments of this aspect of the inventive methods, the CDK inhibitor is p21 or p 16. In a particularly preferred embodiment are fibrosarcoma cells, more preferably human fibrosarcoma cells and most preferably human HT1080 fibrosarcoma cell line and derivatives thereof. The product of the reporter gene or an endogenous gene that is induced by the CDK inhibitor is preferably detected using an immunological reagent, by assaying for an activity of the gene product, or by hybridization to a complementary nucleic acid.

In a fifth aspect, the invention provides methods for inhibiting pathogenic consequences of cellular senescence, such as carcinogenesis or age-related diseases, the method comprising the steps of contacting the cell with a compound that inhibits senescence or the pathogenic consequences of senescence as determined using the methods provided in the aforesaid aspects of the invention.

In a sixth aspect, the invention provides compounds that are identified using any of the methods of the invention as disclosed herein.

In a seventh aspect, the invention provides methods for inhibiting or preventing gene expression induction by CDK inhibitors. In preferred embodiments, the methods comprise the step of contacting a cell with a compound identified by the inventive methods for identifying compounds that inhibit or prevent gene expression induction by CDK inhibitors. In preferred embodiments, effective amounts of the compounds are formulated into pharmaceutical compositions using pharmaceutically-acceptable carriers or other agents and administered to an animal, most preferably an animal suffering from a disease caused by CDK inhibitor-induced gene expression. In preferred embodiments, the disease is cancer, Alzheimer's disease, renal disease, arthritis or atherosclerosis. In preferred embodiments, the methods employ compounds that are NFκB inhibitors.

Specific preferred embodiments of the present invention will become evident from the following more detailed description of certain preferred embodiments and the claims.

### BRIEF DESCRIPTION OF THE DRAWINGS

FIG. 1 is a schematic diagram of the IPTG-regulated retroviral vector LNp21CO3 used to produce the human HT1080 fibrosarcoma cell line variant p21-9.

FIG. 2A is a graph of the time course of p21 induction after the addition of 50 μM IPTG, where p21 levels were determined by ELISA.

FIG. 2B is a graph of the time course of p21 decay after removal of IPTG.

FIG. 3A are photographs of gel electrophoresis patterns of RT-PCR experiments (left), northern blot analysis of cellular mRNA expression (middle) and immunoblotting assays for IPTG-induced changes in expression of the denoted genes; C: control untreated p21-9 cells; I: cells treated for 3 days with 50 μM IPTG. β2-microglobulin (β2-M) was used as a normalization control for RT-PCR and S14 ribosomal protein gene for northern hybridization.

FIG. 3B are photographs of gel electrophoresis of RT-PCR experiments (left) and immunoblotting analysis (right) showing the time course of changes in the expression of the denoted p21 -inhibited genes upon IPTG addition and release.

FIG. 3C are photographs of gel electrophoresis patterns of RT-PCR experiments (left) and northern hybridization analysis (right) of the time course of changes in the expression of the denoted p21-induced genes upon IPTG addition.

FIG. 3D is a comparison of gene expression in untreated control p21-9 cells (C), serum-starved quiescent cells (Q) and IPTG-treated senescent cells (I).

FIG. 4 is a schematic diagram of the IPTG-regulated retroviral vector LNp16RO2 used to produce the human HT1080 fibrosarcoma cell line variant HT1080/LNp16RO2.

FIG. 5 is a graph of the time course of cell growth of HT1080 3′SS6 cells containing LNp16RO2 (as determined by light scattering at 600 nm) after the addition of 50 μM IPTG; - [symbol] -: untreated; - [symbol] -: IPTG-treated.

FIG. 6 are photographs of gel electrophoresis patterns of RT-PCR experiments using cells containing LNp16RO2 for detecting IPTG-induced changes in expression of the denoted genes; C: control untreated cells; I: cells treated for 3 days with 50 μM IPTG. β-actin was used as a normalization control for RT-PCR.

FIG. 7 illustrates the effects of p21 induction in HT1080 p21-9 cells on the expression of luciferase reporter genes driven by the promoters of the indicated p21-inducible genes. The assays were carried out following transient transfection, after two days (for prosaposin promoter) or three days of culture (for all the other promoters) in the presence or in the absence of 50 μM IPTG. The assays were carried out in triplicate (for prosaposin) or in quadruplicate (for all the other constructs).

FIGS. 8A and 8B are graphs showing IPTG dose dependence of luciferase expression in LuNK4p21 cell line after 24 hrs of IPTG treatment (FIG. 7A) and the time course of luciferase expression upon the addition of 50 μM IPTG (FIG. 7B).

FIG. 9 illustrates the effects of p21 induction in HT1080 p21-9 cells on the expression of luciferase reporter genes driven by the NFκB promoter (A) or by the promoters of the indicated p21-inducible genes (B,C). The promoter-reporter constructs were mixed at a molar ration 1:2 with vectors expressing a dominant inhibitor of NFκB (IKK), C-truncated E1A mutant that inhibits p300/CBP (E1AdeltaC), or non-functional N- and C-truncated version of E1A (E1AdeltaN/C). Luciferase levels were measured after 3 days in the presence or absence of IPTG and normalized by the levels of cellular protein.

FIG. 10 is a bar graph of luciferase activity in LuNK4p21 cells in the presence and absence of IPTG and incubated with different amounts of NSAIDs.

FIG. 11 is a photograph of gel electrophoresis patterns of RT-PCR experiments using LuNK4p21 for detecting inhibition of IPTG-induced changes in expression of the denoted genes by different amounts of sulindac; β-actin was used as a normalization control for RT-PCR.

### DETAILED DESCRIPTION OF THE PREFERRED EMBODIMENTS

This invention provides reagents and methods for identifying genes involved in mediating CDK inhibitor-induced cellular senescence and pathogenic consequences of senescence, and compounds capable of inhibiting senescence and pathogenic consequences of senescence in mammalian cells. Particularly provided are embodiments of such reagents and methods for identifying genes involved in cellular senescence and induced by CDK inhibitors p21 or p16.

For the purposes of this invention, the term “CDK inhibitor” is intended to encompass members of a family of mammalian genes having the biochemical activity of cyclin-dependent kinase inhibition. Explicitly contained in this definition are the CDK inhibitors p27, p15, p14, p18 and particularly p16 and p21, the latter two of which are particularly preferred embodiments of the reagents and methods of this invention.

For the purposes of this invention, reference to “a cell” or “cells” is intended to be equivalent, and particularly encompasses in vitro cultures of mammalian cells grown and maintained as known in the art.

For the purposes of this invention, reference to “cellular genes” in the plural is intended to encompass a single gene as well as two or more genes. It will also be understood by those with skill in the art that effects of modulation of cellular gene expression, or reporter constructs under the transcriptional control of promoters derived from cellular genes, can be detected in a first gene and then the effect replicated by testing a second or any number of additional genes or reporter gene constructs. Alternatively, expression of two or more genes or reporter gene constructs can be assayed simultaneously within the scope of this invention.

For the purposes of this invention, the term “senescence” will be understood to include permanent cessation of DNA replication and cell growth not reversible by growth factors, such as occurs at the end of the proliferative lifespan of normal cells or in normal or tumor cells in response to cytotoxic drugs, DNA damage or other cellular insult.

Senescence can be induced in a mammalian cell in a number of ways. The first is a natural consequence of normal cell growth, either in vivo or in vitro: there are a limited number of cell divisions, passages or generations that a normal cell can undergo before it becomes senescent. The precise number varies with cell type and species of origin (Hayflick & Moorhead, 1961, Exp. Cell Res. 25: 585-621). Another method for inducing senescence in any cell type is treatment with cytotoxic drugs such as most anticancer drugs, radiation, and cellular differentiating agents. See, Chang et al., 1999, Cancer Res. 59: 3761-3767. Senescence also can be rapidly induced in any mammalian cell by transducing into that cell a tumor suppressor gene (such as p53, p21, p16 or Rb) and expressing the gene therein. See, Sugrue et al., 1997, Proc. Natl. Acad. Sci. USA 94: 9648-9653; Uhrbom et al., 1997, Oncogene 15: 505-514; Xu et al., 1997, Oncogene 15: 2589-2596; Vogt et al., 1998, Cell Growth Differ. 9: 139-146

For the purposes of this invention, the term “pathological consequences of senescence” is intended to encompass diseases such as cancer, atherosclerosis, Alzheimer's disease, amyloidosis, renal disease and arthritis.

The reagents of the present invention include any mammalian cell, preferably a rodent or primate cell, more preferably a mouse cell and most preferably a human cell, that can induce expression of a CDK inhibitor gene, most preferably p21 or p 16, wherein such gene is either the endogenous gene or an exogenous gene introduced by genetic engineering. Although the Examples disclose recombinant mammalian cells comprising recombinant expression constructs encoding inducible p21 and p16 genes, it will be understood that these embodiments are merely a matter of experimental design choice and convenience, and that the invention fully encompasses induction of endogenous CDK inhibitor genes such as p21 and p 16.

In preferred embodiments, the invention provides mammalian cells containing a recombinant expression construct encoding an inducible mammalian p21 gene. In preferred embodiments, the p21 gene is human p21 having nucleotide and amino acid sequences as set forth in U.S. Pat. No.5,424,400, incorporated by reference herein. In alternative embodiments, the p21 gene is an amino-terminal portion of the human p21 gene, preferably comprising amino acid residues 1 through 78 of the native human p21 protein (as disclosed in U.S. Pat. No.5,807,692, incorporated by reference) and more preferably comprising the CDK binding domain comprising amino acids 21-71 of the native human p21 protein (Nakanishi et al., 1995, EMBO J. 14: 555-563). Preferred host cells include mammalian cells, preferably rodent or primate cells, and more preferably mouse or human cells. Particularly preferred embodiments are fibrosarcoma cells, more preferably human fibrosarcoma cells and most preferably cells of the human HT1080 fibrosarcoma cell line and derivatives thereof. A most preferred cell line is an HT 1080 fibrosarcoma cell line derivative identified as p21-9, deposited on Apr. 6, 2000 with the American Type Culture Collection, Manassas, Va. U.S.A. under Accession No. PTA 1664.

In alternative preferred embodiments, the invention provides mammalian cells containing a recombinant expression construct encoding an inducible mammalian p16 gene. In preferred embodiments, the p16 gene is human p16 having nucleotide and amino acid sequences as set forth in Serrano et al., 1993, Nature 366: 704-707, incorporated by reference herein. Preferred host cells include mammalian cells, preferably rodent or primate cells, and more preferably mouse or human cells. Particularly preferred embodiments are fibrosarcoma cells, more preferably human fibrosarcoma cells and most preferably cells of the human HT1080 fibrosarcoma cell line and derivatives thereof. A most preferred cell line is an HT 1080 fibrosarcoma cell line derivative identified as HT1080/LNp16RO2, deposited on Oct. 10, 2000 with the American Type Culture Collection, Manassas, Virginia U.S.A. under Accession No. PTA-2580.

Recombinant expression constructs can be introduced into appropriate mammalian cells as understood by those with skill in the art. Preferred embodiments of said constructs are produced in transmissible vectors, more preferably viral vectors and most preferably retrovirus vectors, adenovirus vectors, adeno-associated virus vectors, and vaccinia virus vectors, as known in the art. See, generally, MOLECULAR VIROLOGY: A PRACTICAL APPROACH, (Davison & Elliott, ed.), Oxford University Press: New York, 1993.

In additionally preferred embodiments, the recombinant cells of the invention contain a construct encoding an inducible CDK inhibitor gene, wherein the gene is under the transcriptional control of an inducible promoter. In more preferred embodiments, the inducible promoter is responsive to a trans-acting factor whose effects can be modulated by an inducing agent. The inducing agent can be any factor that can be manipulated experimentally, including temperature and most preferably the presence or absence of an inducing agent. Preferably, the inducing agent is a chemical compound, most preferably a physiologically-neutral compound that is specific for the trans-acting factor. In the use of constructs comprising inducible promoters as disclosed herein, expression of CDK inhibitor from the recombinant expression construct is mediated by contacting the recombinant cell with an inducing agent that induces transcription from the inducible promoter or by removing an agent that inhibits transcription from such promoter. In preferred embodiments of this aspect of the inventive methods, the CDK inhibitor is p21 or p 16. A variety of inducible promoters and cognate trans-acting factors are known in the prior art, including heat shock promoters than can be activated by increasing the temperature of the cell culture, and more preferably promoter/factor pairs such as the tet promoter and its cognate tet repressor and fusions thereof with mammalian transcription factors (as are disclosed in U.S. Pat. Nos. 5,654,168, 5,851,796, and 5,968,773), and the bacterial lac promoter of the lactose operon and its cognate lacI repressor protein. In a preferred embodiment, the recombinant cell expresses the lacI repressor protein and a recombinant expression construct encoding human p21 under the control of a promoter comprising one or a multiplicity of lac-responsive elements, wherein expression of p21 can be induced by contacting the cells with the physiologically-neutral inducing agent, isopropylthio-β-galactoside. In this preferred embodiment, the lacI repressor is encoded by a recombinant expression construct identified as 3′SS (commercially available from Stratagene, LaJolla, Calif.). In alternative preferred embodiments, the recombinant cell expresses the lacI repressor protein and a recombinant expression construct encoding human p16 under the control of a promoter comprising one or a multiplicity of lac-responsive elements, wherein expression of p16 can be induced by contacting the cells with the physiologically-neutral inducing agent, isopropylthio-β-galactoside. In this preferred embodiment, the lacI repressor is encoded by the 3′SS recombinant expression construct (Stratagene).

The invention also provides recombinant expression constructs wherein a reporter gene is under the transcriptional control of a promoter of a gene whose expression is modulated by a CDK inhibitor such as p21 or p 16. These include genes whose expression is induced by CDK inhibitors. In preferred embodiments of this aspect of the invention, the CDK inhibitor is p21 or p 16. In preferred embodiments, the promoters are derived from genes whose expression is induced or otherwise increased by CDK inhibitor expression, and are identified in Table II. Most preferably, the promoter is derived from serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p66^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), prostacyclin stimulating factor (SEQ ID NO.: 18), vascular endothelial growth factor-C (SEQ ID NO. 19) and tissue inhibitor of metalloproteinases- 1 (SEQ ID NO. 20). These reporter genes are then used as sensitive and convenient indicators of the effects of CDK inhibitor gene expression, and enable compounds that inhibit the effects of CDK inhibitor expression in mammalian cells to be easily identified. Host cells for these constructs include any cell in which CDK inhibitor gene expression can be induced, and preferably include cells also containing recombinant expression constructs containing an inducible CDK inhibitor gene as described above. Reporter genes useful in the practice of this aspect of the invention include but are not limited to firefly luciferase, Renilla luciferase, chloramphenicol acetyltransferase, beta-galactosidase, green fluorescent protein, and alkaline phosphatase. Particularly preferred embodiments are fibrosarcoma cells, more preferably human fibrosarcoma cells and most preferably cells of the human HT1080 fibrosarcoma cell line and derivatives thereof. A most preferred cell line is an HT 1080 fibrosarcoma cell line derivative identified as HT1080/LUNK4p21, deposited on May 17, 2001 with the American Type Culture Collection, Manassas, Va. U.S.A. under Accession No. PTA-3381.

In preferred embodiments, cells according to the invention comprise both a first recombinant expression construct encoding a reporter gene under the transcriptional control of a promoter for a mammalian gene whose expression is modulated by a CDK inhibitor, and a second recombinant expression construct encoding a mammalian CDK inhibitor gene, wherein CDK inhibitor expression is experimentally-inducible thereby in the mammalian cell. In preferred embodiments of this aspect of the invention, the CDK inhibitor is p21 or p16. In alternative embodiments, the invention provides a mammalian cell comprising a recombinant expression construct encoding a reporter gene under the transcriptional control of a promoter for a mammalian gene whose expression is induced by a CDK inhibitor, wherein the promoter is from the gene encoding connective tissue growth factor serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p66^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), prostacyclin stimulating factor (SEQ ID NO.: 18), vascular endothelial growth factor-C (SEQ ID NO. 19) and tissue inhibitor of metalloproteinases-1 (SEQ ID NO.20). In preferred embodiments of this aspect of the invention, the CDK inhibitor is p21 or p16.

The invention also provides screening methods for identifying compounds that inhibit CDK inhibitor-induced expression of mitogenic or anti-apoptotic factors in mammalian cells. In preferred embodiments, CDK inhibitor expression is induced in a mammalian cell culture in the presence or absence of compounds to be identified as inhibitors of CDK inhibitor-induced expression of mitogenic or anti-apoptotic factors. Compounds are identified as inhibitors by inducing expression of CDK inhibitor in the cells, and comparing the extent of expression of a mitogenic or anti-apoptotic factor, or a plurality thereof, in the presence of the compound with expression in the absence of the compound, and inhibitors identified as compounds that have a reduced amount of expression of a mitogenic or anti-apoptotic factor, or a plurality thereof, in the presence of the compound. In preferred embodiments of this aspect of the invention, the CDK inhibitor is p21 or p16. Any CDK inhibitor-expressing cell is useful for the production of said conditioned media, and CDK inhibitor expression in such cells can be achieved by inducing endogenous CDK inhibitors (such as by treatment with DNA damaging agents and other cytotoxic compounds, and ionizing or ultraviolet radiation, or contact inhibition) or by using cells containing an inducible CDK inhibitor expression construct according to the invention and culturing the cells in a physiologically-neutral inducing agent. In preferred embodiments of this aspect of the invention, the CDK inhibitor is p21 or p16. Preferred cells include mammalian cells, preferably rodent or primate cells, and more preferably mouse or human cells. Particularly preferred embodiments are fibrosarcoma cells, more preferably human fibrosarcoma cells and most preferably cells of the human HT1080 fibrosarcoma cell line and derivatives thereof. An exemplary cell line according to this particularly preferred embodiment of the invention is an HT 1080 fibrosarcoma cell line derivative identified as p21-9, deposited on Apr. 6, 2000 with the American Type Culture Collection, Manassas, Va. U.S.A. under Accession No. PTA 1664. Another exemplary cell line according to this particularly preferred embodiment of the invention is an HT 1080 fibrosarcoma cell line derivative identified as HT1080/LNp16RO2, deposited on Oct. 10, 2000 with the American Type Culture Collection, Manassas, Va. U.S.A. under Accession No. PTA-2580.

In alternative embodiments, the invention provides methods for identifying compounds that inhibit CDK inhibitor-induced expression of mitogenic or anti-apoptotic factors in mammalian cells, wherein the cells comprise a recombinant expression construct encoding a reporter gene under the transcriptional control of a promoter of a cellular gene that is induced by a CDK inhibitor. In preferred embodiments of this aspect of the invention, the CDK inhibitor is p21 or p16. Preferred promoters include the promoters for connective tissue growth factor (CTGF; SEQ ID NO. 3), activin A (SEQ ID NO. 5), epithelin/granulin (SEQ ID NO. 11), galectin-3 (SEQ ID NO. 9), prosaposin (SEQ ID NO. 7), clusterin (SEQ ID NO. 17), prostacyclin stimulating factor (SEQ ID NO.: 18), vascular endothelial growth factor-C (SEQ ID NO.: 19) and tissue inhibitor of metalloproteinases (SEQ ID NO.: 20). Preferred reporter genes include but are not limited to firefly luciferase, Renilla luciferase, β-galactosidase, alkaline phosphatase and green fluorescent protein, all of which are commercially available. In these embodiments, CDK inhibitor expression is induced in the cells, and the extent of expression of the reporter gene is compared in the presence of the compound with expression in the absence of the compound. Inhibitors are identified as compounds that provide a reduced amount of expression of the reporter gene in the presence of the compound than in the absence of the compound. Any CDK inhibitor-expressing cell is useful in this aspect of the invention, and CDK inhibitor expression in such cells can be achieved by inducing the endogenous inhibitor gene (for example, by treatment with DNA damaging agents or other cytotoxic compounds, ionizing or ultraviolet radiation, or contact inhibition) or by using cells containing an inducible CDK inhibitor expression construct according to the invention and culturing the cells in a physiologically-neutral inducing agent. In preferred embodiments of this aspect of the invention, the CDK inhibitor is p21 or p16. Preferred cells include mammalian cells, preferably rodent or primate cells, and more preferably mouse or human cells. A particularly preferred embodiment is fibrosarcoma cells, more preferably human fibrosarcoma cells and most preferably human HT1080 fibrosarcoma cell line and derivatives thereof. A most preferred cell line is an HT1080 fibrosarcoma cell line derivative identified as HT1080/LUNK4p21, deposited on May 17, 2001 with the American Type Culture Collection, Manassas, Va. U.S.A. under Accession No. PTA-3381.

The invention provides methods for identifying compounds that inhibit pathogenic consequences of cell senescence, whereby the effects of the compound are assayed by determining whether the compounds inhibit induction of genes whose expression is induced by a CDK inhibitor. In the practice of the methods of the invention, cultured mammalian cells in which a CDK inhibitor can be induced are treated to induce the inhibitor gene, for example, by ionizing or ultraviolet radiation, or contact inhibition treatment or treatment with cytotoxic drugs, or transduced with a transmissible vector encoding a CDK inhibitor. In preferred embodiments of this aspect of the invention, the CDK inhibitor is p21 or p16. More preferably, p21-9 cells are used in which p21 can be induced by contacting the cells with IPTG (deposited on Apr. 6, 2000 with the American Type Culture Collection, Manassas, Va. U.S.A. under Accession No. PTA 1664), or HT1080/LNp16RO2 cells (deposited on Oct. 10, 2000 with the American Type Culture Collection, Manassas, Va. U.S.A. under Accession No. PTA-2580) are used in which p16 can be induced with IPTG. Typically, cells are grown in appropriate culture media (e.g., DMEM supplemented with 10% fetal calf serum (FCS) for p21-9 cells). In p21-9 cells, p21 gene expression is induced by adding IPTG to the culture media at a concentration of about 50 μM. Typically, the CDK inhibitor is induced in these cells in the presence or absence of the compound to be tested according to the methods of the invention. mRNA is then isolated from cells in which the CDK inhibitor is induced, and expression of genes that are regulated by CDK inhibitors is analyzed. Expression is compared in cells in which the CDK inhibitor is induced in the presence of the compound with expression induced in the absence of the compound, and the differences used to identify compounds that affect cellular gene expression according to the methods set forth herein. In certain embodiments, cellular gene expression is analyzed using microarrays of oligonucleotides or cellular cDNAs such as are commercially available (for example, from Genome Systems, Inc., St. Louis, Mo.). In alternative embodiments, genes known to be induced by CDK inhibitors are assayed. Gene expression can be assayed either by analyzing cellular mRNA or protein for one or a plurality of CDK inhibitor-modulated genes. In preferred embodiments of this aspect of the invention, the CDK inhibitor is p21 or p16. Most preferably, the genes used in these assays are genes identified in Table II.

In alternative embodiments, such compounds are identified independently of CDK inhibitor-directed experimental manipulation. In such assays, cells are treated to induce senescence in any of the ways disclosed above, including but not limited to treatment with cytotoxic drugs, radiation or cellular differentiating agents, or introduction of a tumor suppressor gene. Expression of genes that are induced by CDK inhibitors is analyzed in the presence or absence of the test compound. Most preferably, the genes used in these assays are genes identified in Table II, using the types of mRNA and protein assays discussed above for gene expression analysis.

In alternative embodiments, the cells in which a CDK inhibitor is induced further comprise a recombinant expression construct encoding a reporter gene under the transcriptional control of a promoter of a cellular gene that is induced by a CDK inhibitor. In preferred embodiments of this aspect of the invention, the CDK inhibitor is p21 or p16. In preferred embodiments, the cellular gene is a gene that is induced by the CDK inhibitor, and the promoter is derived from a gene identified in Table II. Examples of known promoters for such genes include serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p66^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), prostacyclin stimulating factor (SEQ ID NO.: 18), vascular endothelial growth factor-C (SEQ ID NO. 19) and tissue inhibitor of metalloproteinases-1 (SEQ ID NO. 20). Preferred reporter genes include but are not limited to firefly luciferase, Renilla luciferase, β-galactosidase, alkaline phosphatase and green fluorescent protein, all of which are commercially available.

The invention also provides methods for identifying genes associated with cellular senescence and pathogenic consequences of senescence or that mediate the effects of CDK inhibitor-induced cellular senescence. Induction of CDK inhibitors turns out to be an integral part of cell growth arrest associated with senescence, terminal differentiation and response to cellular damage. As described in the Examples below, cDNA array hybridization showed that these effects were due to p21-induced changes in gene expression. p21 selectively induced genes that have been associated with cellular senescence and aging or have been implicated in age-related diseases, including atherosclerosis, Alzheimer's disease, amyloidosis, renal disease and arthritis. These findings suggested that cumulative effects of p21 induction in an organism may contribute to the pathogenesis of cancer and age-related diseases. In addition, a number of p21 -activated genes encode secreted proteins with potential paracrine effects on cell growth and apoptosis. In agreement with this observation, conditioned media from p21-induced cells showed mitogenic and anti-apoptotic activity.

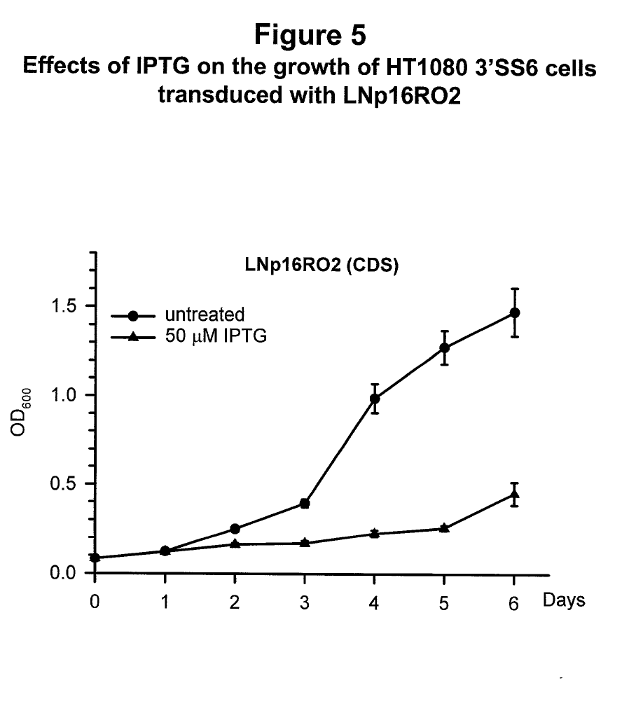

In addition, the results presented in the Examples below demonstrated that induced expression of p16 mimicked the effects of p21 gene expression, and that the same genes whose expression was modulated by p21 gene expression were also modulated by p16 gene expression (see FIG. 6). Thus, the methods of the invention have been extended to include cells in which p16 gene expression is induced, either by induction of the endogenous p16 gene or in recombinant cells comprising an inducible expression construct encoding p16.

The observed effects of CDK inhibitor induction, particularly p21 and p16 induction on gene expression show numerous correlations with the changes that have been associated with cell senescence and organism aging. Some of these correlations come from the analysis of genes that are inhibited by CDK inhibitors. Thus, senescent fibroblasts were reported to express lower levels of Rb (Stein et al., 1999, Mol. Cell. Biol. 19: 2109-2117), as was observed upon p21 induction. It is also interesting that three genes that are inhibited by CDK inhibitors, CHL1, CDC21 and RAD54 encode members of the helicase family. A deficiency in another protein of the helicase group has been identified as the cause of Werner syndrome, a clinical condition associated with premature aging and, at the cellular level, accelerated senescence of cells in culture (Gray et al., 1997, Nature Genet. 17: 100-103).

The strongest correlations with the senescent phenotype, however, come from identification of CDK inhibitor-induced genes, many of which are known to increase their levels during replicative senescence or organism aging. Overexpression of extracellular matrix (ECM) proteins is a known hallmark of replicative senescence, and two CDK inhibitor-induced genes in this group, fibronectin 1 and plasminogen activator inhibitor 1 (PAI-1), have been frequently associated with cellular senescence (reviewed in Crisofalo & Pignolo, 1996, Exp. Gerontol. 31: 111-123). Other CDK inhibitor-induced genes that were also reported to be overexpressed in senescent fibroblasts include tissue-type plasminogen activator (t-PA; West et al., 1996, Exp. Gerontol. 31: 175-193), cathepsin B (diPaolo et al., 1992, Exp. Cell Res. 201: 500-505), integrin β3 (Hashimoto et al., 1997, Biochem. Biophys. Res. Commun. 240: 88-92) and APP (Adler et al., 1991, Proc. Natl. Acad. Sci. USA 88: 16-20). Expression of several CDK inhibitor-induced proteins was shown to correlate with organism aging, including t-PA and PAI-1 (Hashimoto et al., 1987, Thromb. Res. 46: 625-633), cathepsin B (Bernstein et al., 1990, Brain Res. Bull. 24: 43-549) activin A (Loria et al., 1998, Eur. J. Endocrinol. 139: 487-492), prosaposin (Mathur et al., 1994, Biochem. Mol. Biol. Int. 34: 1063-1071), APP (Ogomori et al., 1988, J. Gerontol. 43: B157-B162), SAA (Rosenthal & Franklin, 1975, J. Clin. Invest. 55: 746-753) and t-TGase (Singhal et al., 1997, J. Investig. Med. 45: 567-575).

The most commonly used marker of cell senescence is the SA-β-gal activity (Dimri et al., 1995, Proc. Natl. Acad. Sci. USA 92: 9363-9367). This gene is strongly elevated in IPTG-treated p21-9 cells (Chang et al., 1999, Oncogene 18: 4808-4818). SA-β-gal was suggested to represent increased activity and altered localization of the lysosomal β-galactosidase (Dimri et al., 1995, ibid.), and other studies have described elevated lysosome activities in senescent cells (Cristofalo & Kabakijan, 1975, Mech. Aging Dev. 4: 19-28). Five lysosomal enzymes appear in Table II, including N-acetylgalactosamine-6-sulfate sulfatase (GALNS), cathepsin B, acid α-glucosidase, acid lipase A and lysosomal pepstatin-insensitive protease. p21 also upregulated genes for mitochondrial proteins SOD2, metazin and 2,4-dienoyl-CoA reductase, which correlates with reports of different mitochondrial genes overexpresssed in senescent cells (Doggett et al., 1992, Mech. Aging Dev. 65: 239-255; Kodama et al., 1995, Exp. Cell Res. 219: 82-86; Kumazaki et al., 1998, Mech. Aging Dev. 101: 91-99).

Strikingly, products of many genes that we found to be induced by both p16 and p21 have been linked to age-related diseases, including Alzheimer's disease, amyloidosis, atherosclerosis and arthritis. Thus, APP gives rise to β-amyloid peptide, the main component of Alzheimer's amyloid plaques. Complement C3 (Veerhuis et al., 1995, Virchows Arch. 426: 603-610) and AMP deaminase (Sims et al., 1998, Neurobiol. Aging 19: 385-391) were also suggested to play a role in Alzheimer's disease. It is especially interesting that t-TGase, which is most rapidly induced by p21 and which has been described as a pleiotropic mediator of cell differentiation, carcinogenesis, apoptosis and aging (Park et al., 1999, J. Gerontol. A Biol. Sci. 54: B78-B83), is involved in the formation of plaques associated with both Alzheimer's disease and amyloidosis (Dudek & Johnson, 1994, Brain Res. 651: 129-133). The latter disease is due to the deposition of another CDK inhibitor-induced gene product, SAA, which has also been implicated in atherosclerosis, osteoarthritis and rheumatoid arthritis (Jensen & Whitehead, 1998, Biochem. J. 334: 489-503). Two other CDK inhibitor upregulated secreted proteins, CTGF and galectin 3 are involved in atherosclerosis (Oemar et al., 1997, Circulation 95: 831-839; Nachtigal et al., 1998, Am. J. Pathol. 152: 1199-1208). In addition, cathepsin B (Howie et al., 1985, J. Pathol. 145: 307-314), PAI-1 (Cerinic et al., 1998, Life Sci. 63: 441-453), fibronectin (Chevalier, 1993, Semin. Arthritis Rheum. 22: 307-318), GALNS and Mac-2 binding protein (Seki et al., 1998, Arthritis Rheum. 41: 1356-1364) have been associated with osteoarthritis and/or rheumatoid arthritis. Furthermore, senescence-related changes in ECM proteins, such as increased PAI-1 expression, were proposed to result in age-specific deterioration in the structure of skin and other tissues (Campisi, 1998, J. Investig. Dermatol. Symp. Proc. 3: 1-5). Increased fibronectin production by aging cells was also suggested to increase the density of the fibronectin network in ECM, which may contribute to slower wound healing in aged individuals (Albini et al., 1988, Coll. Relat. Res. 8: 23-37).

p21 and p21-inducible genes have also been implicated in diabetic nephropathy and chronic renal failure. Kuan et al. (1998, J. Am. Soc. Nephrol. 9: 986-993) found that p21 is induced under the conditions of glucose-induced mesangial cell hypertrophy, an in vitro model of diabetic nephropathy. Megyesi et al. (1996, Am. J. Physiol. 271: F1211-1216) demonstrated that p21 is induced in vivo in several animal models of acute renal failure, and this p21 induction is independent of p53. The functional role of p21 in these pathogenic processes has been demonstrated by Al-Douahji et al. (1999, Kidney Int. 56: 1691-1699), who found that p21(−/−) mice do not develop glomerular hypertrophy under the conditions of experimental diabetes, and by Megyesi et al. (1999, Proc Natl Acad Sci USA. 96:10830-10835), who showed that p21(−/−) mice do not develop chronic renal failure after partial renal ablation. Remarkably, Murphy et al. (1999, J. Biol. Chem. 274: 5830-5834), working with the same in vitro model used by Kuan et al. (1998, J. Am. Soc. Nephrol. 9: 986-993), reported that mesangial cell hypertrophy involves upregulation of several genes that we have discovered to be inducible by p21. These include CTGF, fibronectin and plasminogen activator inhibitor 1. The latter study also showed that CTGF plays a functional role in mesangial matrix accumulation in this model system (Murphy et al., 1999, J. Biol. Chem. 274: 5830-5834). These results implicate p21 and p21-mediated induction of gene expression in the pathogenesis of renal failure.

Of special interest, p21 induced the expression of p66^{shc}, a gene recently found to potentiate oxidative damage, with p66(−/−) mice showing increased stress resistance and significantly extended lifespan (Migliaccio et al., 1999, Nature 402: 309-313). These observations suggest that the effects of p21 on gene expression may contribute to the pathogenesis of multiple diseases and overall restriction of the mammalian lifespan.

A major new class of anticancer drugs undergoing clinical trials is angiogenesis inhibitors. These agents target not the tumor cells, but rather the growth of stromal capillaries, stimulated by tumor-secreted angiogenic factors (see Kerbel, 2000, Carcinogenesis 21:505-515,for a recent review). The vasculature, however, is not the only stromal element required for tumor growth. It has been shown in multiple studies that stromal fibroblasts also support the growth of tumor cells in vitro and in vivo, and that normal and immortalized fibroblasts secrete paracrine factors that promote the tumorigenicity and inhibit the death of carcinoma cells (Gregoire and Lieubeau, 1995, Cancer Metastasis Rev. 14: 339-350; Camps et al., 1990, Proc. Natl. Acad. Sci. U.S.A. 87: 75-79; Noel et al., 1998, Int. J. Cancer 76: 267-273; Olumi et al., 1998, Cancer Res. 58: 4525-4530). Such factors have been identified in fibroblast-conditioned media (Chung, 1991, Cancer Metastasis Rev 10: 263-74) and in coculture studies. In particular, Olumi et al. (1998, Cancer Res. 58: 4525-4530) showed that coculture of prostate carcinoma cells with normal prostate fibroblasts strongly decreases carcinoma cell death and promotes xenograft tumor formation. The paracrine effects of fibroblasts also have a tumor-promoting activity in carcinogenesis, as has been demonstrated for initiated prostate epithelial cells (Olumi et al., 1999, Cancer Res. 59: 5002-5011). To the best of our knowledge, this paracrine carcinogenic and tumor-stimulating activity of tumor-associated fibroblasts has not yet been exploited as a target for pharmacological intervention. However, the present invention provides methods for detecting and identifying compounds capable of inhibiting mitogen production from such stromal fibroblasts, thus providing a way to inhibit tumor cell growth.

This paracrine tumor-promoting activity was recently shown to be selectively increased during replicative senescence of normal human fibroblasts (Krtolica et al., 2000, Proc. Amer. Assoc. Can. Res. 41, Abs. 448), a process that involves the induction of p21 and p16. The tumor-promoting effect of the stromal tissue was also shown in a mouse mammary carcinogenesis model to be induced by ionizing radiation (Barcellos-Hoff and Ravani, 2000, Cancer Res. 60: 1254-60), a treatment that produces high p21 levels in stromal fibroblasts (Meyer et al., 1999, Oncogene 18: 5795-5805). These results indicate that the paracrine anti-apoptotic and mitogenic activities that we have discovered in the conditioned media of p21-overexpressing cells are most likely to represent the same biological phenomenon.

The results disclosed herein indicate that CDK inhibitor induction affects cellular gene expression in a way that may increase the probability of the development of cancer or age-related diseases. A surge of CDK inhibitor expression occurs not only in normal replicative senescence but also in response to cellular damage; in both cases, the undesirable effects of CDK inhibitor induction would be expected to accumulate in an age-dependent manner.

Thus, the invention provides methods for identifying compounds that can inhibit induction of genes associated with the pathogenic consequences of cellular senescence, particularly genes that are induced during senescence, and particularly genes that are induced by CDK inhibitor expression. Such compounds would be expected to exhibit the capacity to prevent, retard or reverse age-related diseases by their effects on CDK inhibitor-mediated induction of gene expression.

In one embodiment this invention provides methods for inhibiting gene expression induced by CDK inhibitors such as p21 or p16. In preferred embodiments, such inhibiting is achieved by contacting cells with an effective amount of a compound that inhibits activity, expression or nuclear translocation of nuclear factor kappa-B (NFκB). It will be understood by those with skill in the art that NFκB activity can be inhibited in cells in at least three ways: first, down-regulating or inhibiting transcription, processing and/or translation of either of the genes making up the NFκB heterodimer; second, inhibiting translocation of NFκB from the cytoplasm to the nucleus, which can depend on inhibiting inactivation of IκB expression and/or activity in cells; and third, by inhibiting the activity of NFκB itself. This invention encompasses methods for inhibiting NFκB activity, and thereby inhibiting induction of genes by CDK inhibitors, in any and all of these ways. Examples of NFκB inhibitors known in the art include N-heterocycle carboximide derivatives (as disclosed, for example, in International Application Publication No. WO01/02359); anilide compounds (as disclosed, for example, in International Application Publication No. WO00/15603); 4-pyrimidinoaminoindane derivatives (as disclosed, for example, in International Application Publication No. WO00/05234); 4H-1-benzopyran-4-one derivatives (as disclosed, for example, in Japanese Application No. JP11193231); xanthine derivatives (as disclosed, for example, in Japanese Application No. JP9227561); carboxyalkenylkbenzoquinone and carboxyalkenylnaphthol derivatives (as disclosed,for example, in Japanese Application No. JP7291860); disulfides and derivatives thereof (as disclosed, for example, in International Application Publication No. WO99/40907); protease inhibitors (as disclosed,for example, in European Application Publication No. EP652290); flurbiprofen, thalidomide, dexamethasone, pyrrolidine dithiocarbamate, dimethylfumarate, mesalizine, pimobendan, sulfasalazine, methyl chlorogenate, chloromethylketone, alpha-tocopherol succinate, tepoxaline, and certain non-steroidal anti-inflammatory drugs (NSAIDs), including aspirin, sodium salicylate and sulindac

The following Examples are intended to further illustrate certain preferred embodiments of the invention and are not limiting in nature.

### EXAMPLE 1

### Production of a Mammalian Cell Comprising an Inducible p21 Gene

A recombinant derivative of human fibrosarcoma cell line HT1080, p21-9, was produced essentially according to Chang et al. (1999, Oncogene 18: 4808-4818, incorporated by reference herein). This cell line contained a p21 coding sequence under the transcriptional control of a promoter regulated by isopropyl-β-thiogalactoside (IPTG). Expression of p21 can be induced by culturing these cells in the presence of a sufficient amount of IPTG, thereby permitting the sequellae of p21 expression to be studied in the absence of any additional effects that induction of the endogenous p21 gene might provoke. This cell line has been deposited on Apr. 6, 2000 in the American Type Culture Collection (A.T.C.C.), Manassas, Va. and given Accession Number PTA 1664.

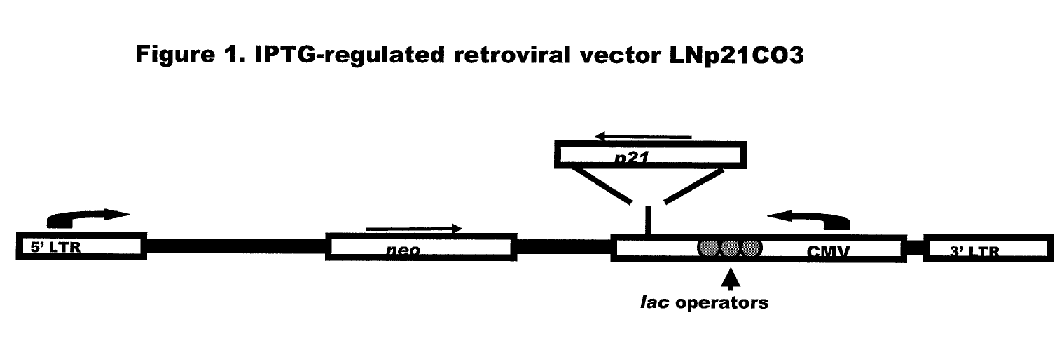

Briefly, a subline of HT1080 expressing a murine ecotropic retrovirus receptor and a modified bacterial lacI repressor encoded by the plasmid 3′SS (Stratagene) (described in Chang & Roninson, 1996, Gene 33: 703-709, incorporated by reference) was infected with retroviral particles containing recombinant retrovirus LNp21 CO3, the structure of which is shown in FIG. 1. This retroviral vector contains the bacterial neomycin resistance gene (neo) under the transcriptional control of the retroviral long terminal repeat promoter. p21-encoding sequences are cloned in the opposite orientation to the transcriptional direction of the neo gene, and under the control of a modified human cytomegalovirus promoter. Specifically, the CMV promoter contains a three-fold repeat of bacterial lac operator sequences that make expression from the promoter sensitive to the lacI repressor expressed in the cell. LNp21CO3 was constructed by cloning a 492bp fragment of DNA comprising the p21 coding sequence into the NotI and BglII sites of the parent vector, LNXCO3 (disclosed in Chang & Roninson, ibid.).

After infection, cells infected with the LNp21CO3X vector were selected by culturing the cells in the presence of 400 μg/mL G418 (obtained from BRL-GIBCO, Gaithersburg, Md.). Clonal line p21-9 was derived from LNp21CO3 transduced, G418-resistant cell lines by end-point dilution until a clonal cell line was obtained.

### EXAMPLE 2

### Cell Growth Assays

p21-9 cells produced as described in Example 1 were used in cell growth assays to determine what changes in cell growth occurred when p21 was expressed in the cell.

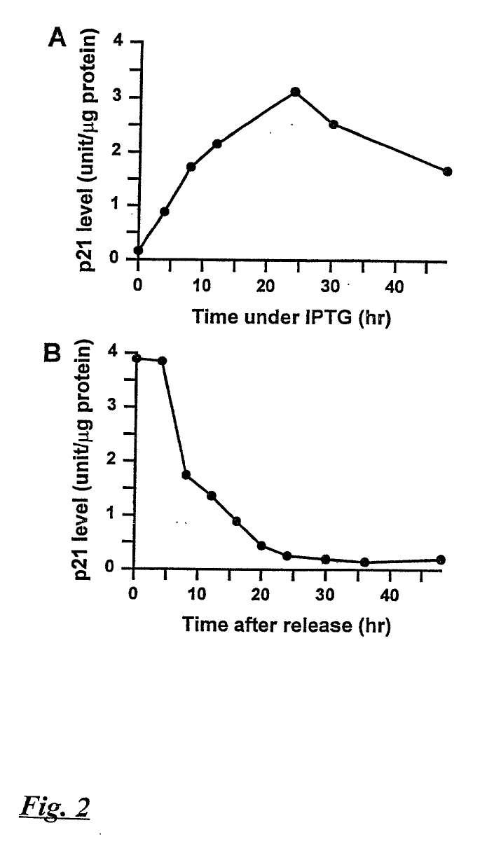

p21 expression from the LNp21CO3 vector in p21-9 cells was induced by culturing the cells in DMEM medium containing 10% fetal calf serum (Hyclone, Logan, Utah) and IPTG. Results of these assays are shown in FIGS. 2A and 2B. FIG. 2A shows the time course of p21 protein production in cells cultured in the presence of 50 μM IPTG. p21 gene expression increased between 6 and 12 hours after introduction of IPTG into the growth media, which expression peaked at about 24 hours post-induction. Upon removing the cells from IPTG-containing media, p21 expression fell about as rapidly as it had risen, returning to pre-induction levels at about 24 hours after IPTG was removed (FIG. 2B).

Cell growth in the presence of IPTG was assayed in three ways: measuring ³H-thymidine incorporation (termed the “labeling index”); observing the number of mitotic cells in the culture by microscopy (termed the “mitotic index”) and determining the distribution of the culture cells in different portions of the cell cycle (termed the “cell cycle distribution”).

³H-thymidine incorporation assays were performed substantially as described by Dimri et al. (1995, Proc. Natl. Acad. Sci. USA 92: 9363-9367). Cells were cultured in the presence of ³H-thymidine for 3h, and then analyzed by autoradiography. DNA replication was determined by autoradiography ceased entirely by 9 hours after addition of IPTG to the culture media. The mitotic index was determined by observing cells microscopically and calculating the number of cells in mitosis after staining with 5 μg/mL 4,6-diamino-2-phenylindole (DAPI), and images were collected using a Leica DMIRB fluorescence microscope and Vaytek (Fairfield, Iowa) imaging system. Microscopically-detectable mitotic cells disappeared from these cultures by 14 hrs of IPTG treatment.

Cell cycle distribution was determined using FACS analysis of DNA content after staining with propidium iodide as described by Jordan et al. (1996, Cancer Res. 56: 816-825) using Becton Dickinson FACSort. Cell cycle distribution stabilized after 24 hrs of IPTG treatment. By this time, 42-43% of IPTG-treated cells were arrested in G1 and G2, respectively, and about 15% of the cells were arrested with S-phase DNA content. IPTG-treated p21-9 cells also developed morphological senescence markers (enlarged and flattened morphology and increased granularity), as well as SA-β-gal activity (Chang et al, 1999, ibid.). These results indicated that induced expression of p21 produces both cell cycle arrest and a variety of other changes that are characteristic of cell senescence.

### EXAMPLE 3

### Analysis of Gene Expression Modulated by p21 Gene Expression

The results disclosed in Example 2 suggested that the morphological and cell cycle consequences of p21 induction could reflect multiple changes in gene expression. The effects of p21 induction on cellular gene expression were examined as follows.

Poly(A)⁺ RNA was isolated from untreated p21-9 cells and from cells that were treated for 3 days with 50 μm IPTG. cDNA was prepared from the poly(A)⁺ RNA and used as probes for differential hybridization with the Human UniGEM V cDNA microarray (as performed by Genome Systems, Inc., St. Louis, Mo.), which contains over 4,000 sequence-verified known human genes and 3,000 ESTs. More than 2,500 genes and ESTs showed measurable hybridization signals with probes from both untreated and IPTG-treated p21-9 cells. Genes that were downregulated with balanced differential expression≧2.5 or upregulated with balanced differential expression≧2.0 are listed in Tables I and II, respectively.

Expression of 69 of these genes was individually tested by RT-PCR or northern hybridization. RT-PCR analysis was carried out essentially as described by Noonan et al. (1990, Proc. Natl. Acad. Sci. USA 87: 7160-7164). Probes fornorthern hybridization were derived from inserts of the cDNA clones present in the microarray; these cDNAs were obtained from Genome Systems, Inc. In addition, changes in the expression of several p21 -regulated gene products were analyzed by immunoblotting. The following primary antibodies were used for immunoblotting: mouse monoclonal antibodies against Cdc2 (Santa Cruz), cyclin A (NeoMarkers), Plk 1 (Zymed) and Rb (PharMingen); rabbit polyclonal antibodies against MAD2 (BadCo), p107 (Santa Cruz), CTGF (Fisp-12; a gift of Dr. L. Lau), Prc 1 (a gift of Drs. W. Jiang and T. Hunter), and topoisomerase IIα (Ab0284; a gift of Dr. W. T. Beck), and sheep polyclonal antibody against SOD2 (Calbiochem). Horse radish peroxidase (HRP)-conjugated secondary antibodies used were goat anti-mouse and goat anti-rabbit IgG (Santa Cruz) and rabbit anti-sheep IgG (KPL). Protein concentrations in all samples were equalized after measurement with BioRad protein assay kit. Immunoblotting was carried out by standard procedures, and the signal was detected by chemiluminescence using LumiGlo (KPL).

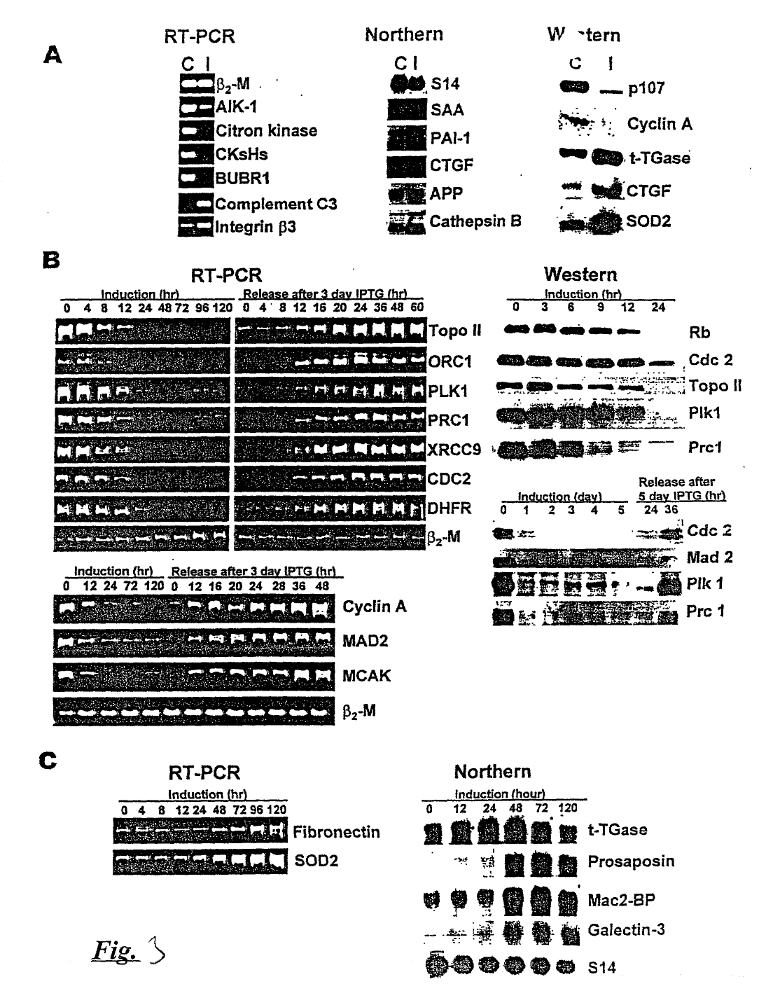

These results are shown in FIGS. 3A through 3C. The changes in gene expression predicted by the microarray assays described above were confirmed for 38/39 downregulated and 27/30 upregulated genes. The observed signal differences in northern hybridization or RT-PCR for most of the tested genes (FIG. 3A through 3C) appeared to be higher than the values of balanced differential expression determined from the cDNA array (Tables I and II), suggesting that cDNA array hybridization tends to underestimate the magnitude of p21 effects on gene expression. Changes in the expression of 6 downregulated and 4 upregulated genes were also tested at the protein level by immunoblotting (FIG. 3B) or zymography (not shown) and were confirmed in all cases tested.

It was recognized that p21 -mediated changes in gene expression were comprised of near-term effects and longer-term effects that followed p21-induced cell growth arrest. For this purpose, the time course of changes in the RNA levels of a subset of p21 -inhibited (FIG. 3B) and p21 -induced genes (FIG. 3C) after the addition and removal of IPTG was determined. Immunoblotting was used to analyze the time course of p21-induced changes in Rb phosphorylation (as indicated by electrophoretic mobility) and in the cellular levels of Rb and several proteins that were inhibited by p21 according to the cDNA array; these results are shown in FIG. 3B. Rb was found to become dephosphorylated as early as 6 hrs after the addition of IPTG. Furthermore, Rb protein levels decreased sharply between 12-24 hrs (shown in FIG. 3B), but no significant changes were detected in RB mRNA levels (data not shown). A similar decrease was observed for a Rb-related protein p107 (shown in FIG. 3A).

1. Gene Expression Inhibited by p21

All the tested p21-inhibited genes showed a rapid response to p21 induction and release. Five of these genes (topoisomerase IIα, ORC1, PLK1, PRC1 and XRCC9) showed significant inhibition at both RNA and protein levels between 4 and 8 hrs after the addition of IPTG (FIG. 3B). This pattern has been termed an “immediate response,” which parallels the kinetics of cell growth arrest and Rb dephosphorylation. Other p21-inhibited genes (such as CDC2 or DHFR) showed an “early response” pattern that lags slightly behind the cessation of DNA replication and mitosis, with a major decrease in mRNA levels detectable only 12 hrs after the addition of IPTG. All p21 -inhibited genes, however, resumed their expression 12-16 hrs after the removal of IPTG, when the cells were still growth-arrested and before the resumption of DNA replication and mitosis (FIG. 3B). This analysis indicated that changes in the expression of p21 -inhibited genes were near-term effects of p21 induction and release and were not a consequence of cell growth arrest and recovery.

In summary, 69 genes and 3 ESTs were identified by the cDNA microarray as downregulated in p21-induced cells, with balanced differential expression of 2.5-12.6 (Table IA); five additional genes that are associated with cell cycle progression and have been identified by our separate assays as downregulated in IPTG-treated cells are listed in Table IB. A strikingly high fraction of downregulated genes identified by the cDNA array (43 of 69) were associated with mitosis, DNA replication, segregation and repair and chromatin assembly, indicating a highly selective nature of p21-mediated inhibition of gene expression.

The largest group of p21-downregulated genes are that have been implicated in the signaling, execution and control of mitosis. Many p21-inhibited genes are involved in DNA replication and segregation, chromatin assembly and DNA repair. Some of these genes encode enzymes involved in nucleotide biosynthesis, other proteins are involved in DNA replication. Several p21-inhibited genes are associated with DNA repair. These results suggest opportunities for discovering components of the cellular program ofp21-induced growth arrest that would be targets for therapeutic intervention.

2. Gene Expression Induced by p21

In addition to genes repressed by p21 expression, the assays described above detected genes induced by p21. The pattern of gene expression of p21-induced genes is shown in FIG. 3C. In contrast to p21-inhibited genes, p21-upregulated genes increased their expression only 48 hrs after the addition of IPTG, i.e. after the onset of growth arrest in all cells. Only one tested gene, tissue transglutaminase (t-TGase), showed a detectable increase 12 hrs after the addition of IPTG, but its expression reached a maximum only by 48 hrs (as shown in FIG. 3C). Furthermore, elevated expression of all the tested genes (except for t-TGase) persisted for at least three days after release from IPTG, well after resumption of the cell cycle (not shown). This “late response” kinetics indicated that p21 induction of such genes was a delayed effect relative to p21 -mediated growth arrest.

48 known genes and 6 ESTs or genes with unknown functions were identified as upregulated in p21 -induced cells, with balanced differential expression of 2.0-7.8 (Table II). A very high fraction (20/48) of identifiable genes in this group encode extracellular matrix (ECM) components (e.g. fibronectin 1, laminin α2, Mac-2 binding protein), other secreted proteins (e.g. activin A, connective tissue growth factor, serum amyloid A), or ECM receptors (such as integrin β3). Several of these secreted proteins, as well as a large group of p21 -induced intracellular proteins (Table II), are known to be induced in different forms of stress response or to play a role in stress-associated signal transduction. Remarkably, many genes that we found to be induced by p21 are also upregulated in cellular senescence, organism aging, or different age-related diseases, indicating that suppression of p21-mediated gene induction may provide a way to prevent the development of such diseases. As disclosed in Example 5 below, several p21 -induced genes encode secreted factors with paracrine anti-apoptotic and mitogenic activities, and conditioned media from p21-induced cells exhibits two biological effects predicted by the nature of p21-upregulated genes: stimulation of cell growth and suppression of apoptosis. This finding, suggests that “paracrine” effects of p21 may contribute to carcinogenesis through a tumor-promoting effect on neighboring cells. This raises the possibility that suppression of p21-mediated gene induction may also provide a way to achieve an anti-carcinogenic effect.

### EXAMPLE 4

### Identifying the Specificity of p21 Induction by Comparing IPTG-treated and Serum-Starved p21-9 Cells

The identity of p21-induced changes in cellular gene expression that are likely to be a consequence of cell growth arrest was determined as follows.

Growth arrest (quiescence) was induced in p21-9 cells by serum starvation produced by culturing the cells in serum-free media for 4 days. In serum-starved cells, unlike IPTG-treated p21-9 cells, the cells did not develop a senescent morphology and showed only very weak SA-β-gal expression. p21 levels in serum-starved cells were increased only about 2-fold, as opposed to the 15-20 fold increase seen in IPTG-treated cells. FIG. 3D shows RT-PCR analysis performed as described above of the expression of a group of p21-inhibited and p21-induced genes in p21-9 cells that were growth-arrested after 4 days in serum-free media or 3 days in the presence of 50 μM IPTG. Genes that were completely inhibited in p21-9 cells when the culture media contained 50 μM IPTG were also inhibited in serum-starved cells, but most of these genes were inhibited to a lesser extent than in IPTG-treated cells.

Genes whose expression is induced by p21 showed three distinct patterns. The first group are genes whose expression is induced as strongly in quiescent cells as in senescent cells. These include galectin-3, superoxide dismutase 2, complement C3 and prosaposin, indicating that their induction was a consequence of cell growth arrest or that such genes were exquisitely sensitive to slightly elevated p21 levels. The second group are genes that were up-regulated in quiescent cells but not as strongly as in senescent cells. These genes include fibronectin-1, Mac2 binding protein and the Alzheimer precursor protein serum amyloid A. The third group are genes that are not detectably induced in quiescent cells but are strongly induced in senescent cells. These genes include CTGF, plasminogen activator inhibitor 1, tissue transglutaminase or natural killer cell marker protein NK4, integrin beta 3 and activin A.

The difference between the response of certain genes to induction of quiescence by serum starvation and cellular senescence through IPTG-induced overexpression of p21 identified these genes as diagnostic markers of senescence. Furthermore, novel senescence markers can now be identified by comparing their expression between p21-expressing and quiescent cells.

### EXAMPLE 5

### Production of Conditioned Media Containing Mitogenic Factors and Mitogenic Activity Assays

Several p21-upregulated genes (Table II) encode secreted proteins that act as growth factors, including CTGF (Bradham et al., 1991, J. Cell Biol. 114: 1285-1294), activin A (Sakurai et al., 1994, J. Biol. Chem. 269: 14118-14122), epithelin/granulin (Shoyab et al., 1990, Proc. Natl. Acad. Sci. USA 87: 7912-7916) and galectin-3 (Inohara et al., 1998, Exp Cell Res. 245: 294-302). In addition, galectin-3 (Akahani et al, 1997, Cancer Res. 57: 5272-5276) and prosaposin (Hiraiwa et al., 1997, Proc. Natl. Acad. Sci. USA 94: 4778-4781) were shown to have anti-apoptotic activity. Paracrine anti-apoptotic or mitogenic activities have also been reported for several p21-inducible gene products that are not listed in Table II, since their balanced differential expression values in cDNA microarray hybridization were 1.8-1.9. This is below the arbitrarily chosen minimum value of 2.0 that we have used for inclusion into this Table or verification by RT-PCR. These proteins are clusterin (Koch-Brandt and Morgans, 1996, Prog. Mol. Subcell. Biol. 16: 130-149), prostacyclin-stimulating factor (PSF) (Yamauchi et al., 1994, Biochem. J. 303: 591-598), vascular endothelial growth factor-C (VEGF-C) (Joukov et al., 1996, EMBO J. 15: 290-298), gelsolin (Ohtsu et al., 1996, EMBO J 16: 4650-4656) and tissue inhibitor of metalloproteinases-1 (TIMP-1) (Li et al., 1999, Cancer Res. 59: 6267-6275).

To verify the induction of secreted mitogenic and anti-apoptotic factors by p21, conditioned media from IPTG-treated p21-9 cells were tested to investigate whether they would have an effect on cell growth and apoptosis. In these experiments, conditioned media were prepared by plating 10⁶ p21-9 cells per 15 cm plate in the presence of DMEM/10% FCS. The next day, IPTG was added to a final concentration of 50 μM, and this media was replaced three days later with DMEM supplemented with 0.5% FCS and 50 μM IPTG. Two days later (days 3-5 of IPTG treatment), this conditioned media was collected and stored at 4° C. up to 15 days before use. Control media were prepared by adding IPTG-free DMEM/0.5% FCS to untreated cells grown to the same density as IPTG-treated cells and collecting the media two days thereafter.

The slow-growing human fibrosarcoma cell line HS 15.T was used to detect mitogenic activity in these conditioned media. For mitogenic activity assays, both types of conditioned media, as well as fresh media and 1:1 mixtures of conditioned media and fresh media were used to test mitogenic activity. In these experiments, the conditioned media were supplemented with 1% or 2% FCS. Briefly, HS 15.T cells were plated in 12-well plates at 15,000 cells per well. Two days later, these cells were cultured in different types of media. The cells were grown in conditioned media for 60 hr, and the ³H-thymidine at a concentration of 3.13 μCi/mL was added and incubated for 24 hrs. Cells were then collected and their ³H-thymidine incorporation determined as described by Mosca et al. (1992, Mol. Cell. Biol. 12: 4375-4383).

The addition of IPTG to fresh media had no effect in this assay. There was no significant difference between cell growth in fresh media and in conditioned media from untreated p21-9 cells. In contrast, conditioned media from IPTG-treated cells increased ³H-thymidine incorporation up to three-fold. Growth stimulation of HS 15.T by conditioned media from IPTG-treated cells was also detectable by methylene blue staining.

The effect of this conditioned media on apoptosis was also determined. These experiments used a mouse embryo fibroblast line C8, immortalized by E1A. This cell line is highly susceptible to apoptosis induced by different stimuli (Lowe et al., 1994, Science 266: 807-810; Nikiforov et al., 1996, Oncogene 13: 1709-1719), including serum starvation (Lowe et al., 1994, Proc. Natl. Acad. Sci. USA 91: 2026-2030). Apoptosis was analyzed by plating 3×10⁵ C8 cells per 6-cm plate, and replacing the media on the following day with fresh media supplemented with 0.4% serum or with conditioned media (no fresh serum added). DNA content analysis and DAPI staining were carried out after 24 hrs and 48 hrs, and relative cell numbers were measured by methylene blue staining (Perry et al., 1992, Mutat. Res. 276: 189-197) after 48 hrs in low-serum media.

The addition of low-serum fresh media or conditioned media from IPTG-treated or untreated cells rapidly induced apoptosis in C8 cells, as evidenced by cell detachment and apoptotic morphology detectable in the majority of cells after DAPI staining (not shown). Conditioned media from IPTG-treated cells, however, strongly increased cell survival relative to fresh media and conditioned media from untreated cells, as measured by methylene blue staining of cells that remained attached after 48 hrs. The effect of the conditioned media from p21 -induced cells was even more apparent in FACS analysis of cellular DNA content, which was carried out on combined attached and floating C8 cells 24 hrs and 48 hrs after media change. Unlike many other cell lines, apoptosis of C8 cells produces only a few cells with decreased (sub-GI) amount of DNA, and it is characterized by selective disappearance of cells with G2/M DNA content (Nikiforov et al., 1996, ibid.). Serum-starved cells in conditioned media from IPTG-treated cells retained the G2/M fraction and showed cell cycle profiles that resembled control cells growing in serum-rich media. The addition of IPTG by itself had no effect on apoptosis in C8 cells. Thus, p21 induction in HT1080 cells results in the secretion of mitogenic and anti-apoptotic factors, as predicted by the nature of p21 -unregulated genes.

### EXAMPLE 6

### Production of a Mammalian Cell Comprising an Inducible p16 Gene

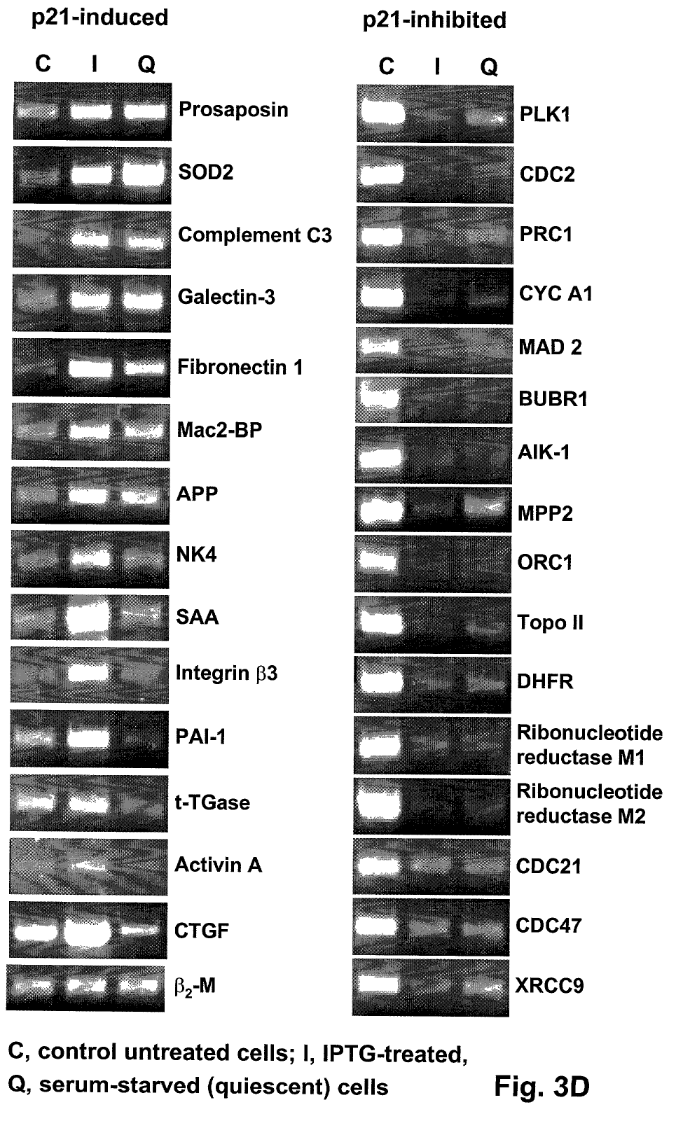

A mammalian cell line comprising an inducible p16 gene was produced generally as described in Example 1 for production of an inducible p21 containing cell line. A recombinant derivative of human HT1080 fibrosarcoma cell line containing a recombinant expression construct encoding the bacterial lacI gene and expressing a murine ecotropic retrovirus receptor (HT1080 3′SS6; Chang & Roninson, 1996, Gene 183:137-142) were used to make the inducible p16-containing cells. A DNA fragment containing a 471bp coding sequence of human p16 (as disclosed in U.S. Pat. No. 5,889,169, incorporated by reference) was cloned into the IPTG-regulated retroviral vector LNXRO2 (Chang & Roninson, 1996, Gene 183: 137-142). This retroviral vector contains the bacterial neomycin resistance gene (neo) under the transcriptional control of the retroviral long terminal repeat promoter, permitting selection using G418 (BRL-GIBCO). The resulting construct, designated LNp16RO2, is depicted schematically in FIG. 4. This construct was introduced into HT1080 3′SS cells using conventional retroviral infection methods. After infection, cells infected with the LNp 16RO2 vector were selected by culturing the cells in the presence of 400 μg/mL G418 (obtained from BRL-GIBCO). The G418-selected population of LNp16RO2 transduced cells was designated HT 1080/LNp 16RO2. This cell population has been deposited on Oct. 10, 2000 in the American Type Culture Collection (A.T.C.C.), Manassas, Va. and given Accession Number PTA-2580.

### EXAMPLE 7

### Cell Growth and Gene Expression Assays

The HT1080 derivatives carrying a human p16 gene inducible with IPTG as described in Example 6 were used in cell growth and gene expression assays as follows.

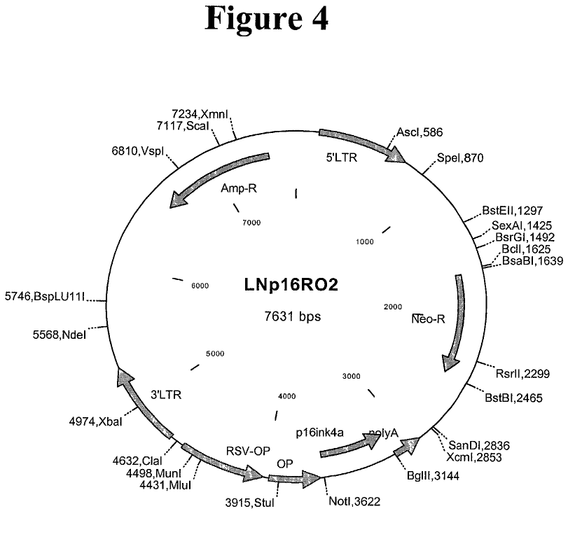

Cells were grown in the presence and absence of 50 μM IPTG over the course of 6 days and the number of cells in the culture determined by light scattering at 600 nm. These results are shown in FIG. 5. Culturing these cells in the presence of 50 μM IPTG, thus inducing p16 gene expression, resulted in growth inhibition relative to cells grown in the absence of IPTG.

RNA was then obtained from these cells, cultured in the presence or absence of 50 μM IPTG for three days. These RNA samples were then used in RT-PCR assays performed essentially as described above in Example 3, except that β-actin rather than β₂-microglobulin was used for normalization. Nine genes shown above to be inhibited by p21 and eighteen genes shown above to be induced by p21 were analyzed for the effects of p16 gene expression induced by IPTG treatment of these cells. These results are shown in FIG. 6. All the tested p21 -inhibited genes were also inhibited by IPTG-induced expression of p16, and all the tested p21-induced genes were also induced by IPTG-induced p16 expression. The tested inhibited genes were genes involved in cell cycle progression, and the tested induced genes were genes involved in Alzheimer's disease, amyloidosis, arthritis, atherosclerosis and paracrine apoptotic and mitogenic effects as described above with regard to induced p21 expression. The results shown in FIG. 6 also illustrate that p16 expression has no detected effect on p21 expression.

### EXAMPLE 8

### Production of Recombinant Expression Constructs Containing a Reporter Gene Expressed by a p21-responsive Promoter

Promoter-reporter constructs were prepared from promoters of several p21-inducible human genes, including NK4, SAA, Complement C3 (CC3), prosaposin, βAPP and t-TGase as follows. The promoter region of the CC3 gene was identified in the human genome sequence (NCBI Accession number M63423.1) as adjacent to the 5′ end of CC3 cDNA (Vik et al., 1991, Biochemistry 3: 1080-1085). The promoter region of the NK4 gene was identified in the human genome sequence (Accession number AJ003147) as adjacent to the 5′ end of NK4 cDNA (Accession number M59807). The previously described promoter of the SAA gene (Edbrooke et al., 1989, Mol. Cell. Biol. 9: 1908-1916) was identified in the human genome sequence (Accession number M26698). The promoter region of the βAPP gene was identified in the human genome sequence (Accession number X12751) as adjacent to the 5′ end of βAPP cDNA (Accession number XM009710). The promoter region of the t-TGase gene was identified in the human genome sequence (Accession number Z46905) as adjacent to the 5′ end of t-TGase CDNA (Accession number M55153). Polymerase chain reaction (PCR) amplification of promoter-specific DNA was performed using genomic DNA from HT1080 p21-9 cells as the template. PCR was carried out using PfuTurbo DNA Polymerase (Stratagene) and primer sets listed in Table IIIa. The PCR conditions for each primer set are described in Table IIIb. Primer sets for amplifying promoter sequences from several genes induced by CDK inhibitors, including the gene promoters used as disclosed in this Examiner, are set forth in Table IIIc.

PCR products were obtained and cloned into the TOPO TA cloning vectors pCR2.1/TOPO (for SAA, CC3, βAPP and t-TGase) or pCRII/TOPO (for NK4). These constructs were verified by sequencing, and the Kpn I-Xho I fragments containing promoters in the correct orientation were then inserted into the Kpn I and Xho I sites in a firefly luciferase-reporter vector pGL2 basic (Promega, Madison, Wis.) using standard recombinant genetic techniques (Sambrook et al., ibid.). The clone containing a 480 bp sequence of the prosaposin promoter, driving firefly luciferase expression has been described by Sun et al. (1999, Gene 218, 37-47) and provided by Dr. Grabowski (Children's Hospital Medical Center, Cincinnati, Ohio).

Plasmid clones for each promoter construct were tested for p21-regulation by a transient transfection assay. Transient transfection of HT1080 p21-9 cells was carried out by electroporation, essentially as described in the Bio-Rad protocols. For each electroporation, p21-9 cells were grown to confluence in 15 cm plates using DMEM supplemented with 10% FC2 serum and containing penicillin, streptomycin and glutamine.. The cells were then trypsinized, resuspended in Opti-MEM medium (GibcoBRL) and spun down at 1,000 rpm in an IEC HN-SII centrifuge for 10 minutes. Following centrifugation the media were aspirated and the cells were again resuspended in Opti-MEM at a concentration of 18-20 million cells per ml. 400 μl of cell suspension (approximately 7 to 8 million cells) was transferred to a 4 cm gap electroporation cuvette (Bio-Rad). 10-20 μg of the promoter-luciferase construct was added to the cells. In some experiments, a control plasmid pCMVbgal expressing bacterial P-galactosidase from the CMV promoter, was added to the mixture at a ratio of 1:10 for normalization. Electroporations were performed using Bio-Rad Gene Pulser at 0.22 volts, with a capacitance extender set to 960 μFD, providing a τ value of 27 to 30. In preliminary experiments, cell survival and attachment after electroporation was determined to be approximately 33%. Cells were plated in triplicate at an initial density of approximately 50,000 attached cells/well in 12-well plates. After letting the cells settle for a period of 3-6 hours, the media was aspirated and replaced with fresh media with or without 50 μM IPTG. 2 or 3 days later, cells were washed twice with phosphate-buffered saline and collected in 300 μL of Reporter Lysis Buffer (Promega). The lysate was centrifuged briefly at 10,000 g to pellet debris, and 50 μL aliquots were transferred to fresh tubes for use in the Firefly Luciferase assay (Promega). Luciferase activity was measured using a Turner 20/20 luminometer at 52.1% sensitivity with a 5 second delay period and 10-15 second integration time.

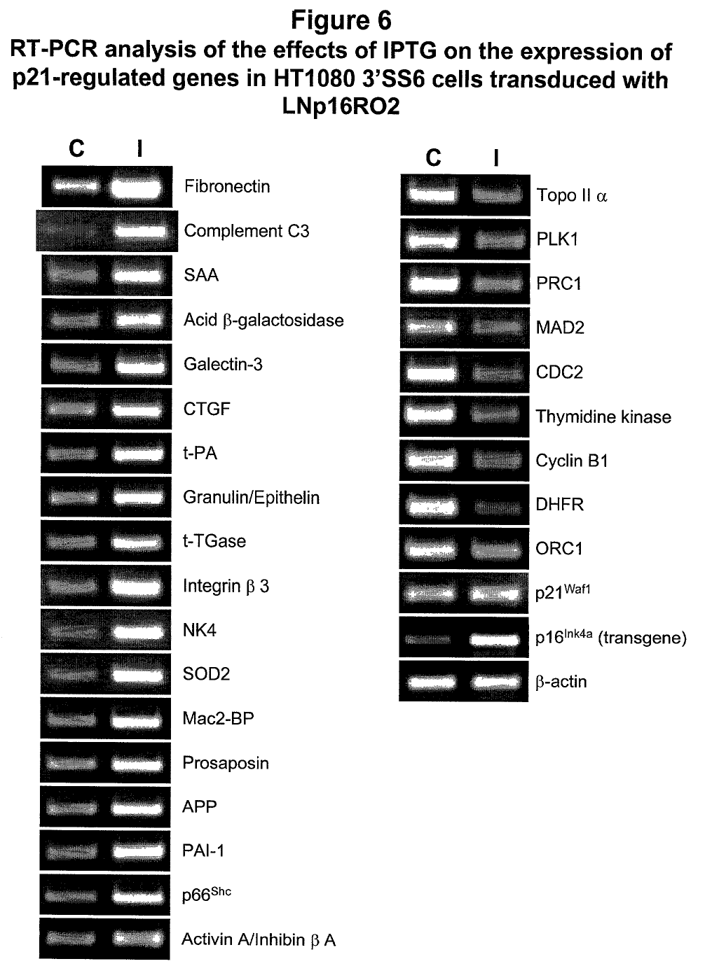

FIG. 7 shows the results of representative experiments. After 2-3 days of p21-induction in transfected cells, expression from promoter constructs of p21-induced genes was increased about 7.0-fold for NK4, 3.7-fold for SAA, 12.5-fold for CC3, 3.0-fold for prosaposin, 2.6-fold for βAPP, and 2.3-fold for t-TGase. These results indicated that p21 up-regulates expression of these genes by regulating their promoters, and that promoter constructs of such genes can be used to assay for p21-mediated regulation of gene expression. Such assays can be used to identify compounds that inhibit p21-mediated gene activation, as described below in Example 9.

TABLE IIIa
Primer sequences

| | | Antisense |
|---|---|---|
| Promoter | Sense primer (5′->3′) | primer (5′->3′) |
| CC3 | GCTAA (SEQ ID NO.21) | AGGGGG (SEQ ID NO.22) |
| | GAGGATATTGACATTAGAC | AGGTGGGTTAGTAG |
| | AGG | |
| NK4 | TGGAG (SEQ ID NO.23) | GCCAAA (SEQ ID NO.24) |
| | CTAGAAGAGCCCGTAGG | AGTTCAAGGAGCCAA |
| SAA | CAGAG (SEQ ID NO.25) | CACTCC (SEQ ID NO.26) |
| | TTGCTGCTATGTCCACCA | TTGTGTGCTCCTCACC |
| βAPP | TTGCT (SEQ ID NO.27) | GCTGCC (SEQ ID NO.28) |
| | CCTTTGGTTCGTTCT | GAGGAAACTGAC |
| t-TGase | CCCAG (SEQ ID NO.29) | TCGGGC (SEQ ID NO.30) |
| | GGAGAAATATCCACTGAAGC | GGGGGCGGTGGCTCCTTCCAC |
| | AAC | T |

TABLE IIIb
PCR conditions

| Pro- | | | | Cy- | Product |
|---|---|---|---|---|---|
| moter | Denaturation | Annealing | Extension | cles | size |
| CC3 | 95°, 1 min | 63°, 1 min | 72°, 1 min 40 sec | 31 | 1018 bp |
| NK4 | 94°, 1 min | 65°, 1 min | 72°, 1 min 40 sec | 32 | 877 bp |
| SAA | 94°, 1 min | 68°, 1 min | 72°, 1 min 40 sec | 32 | 1000 bp |
| βAPP | 94°, 1 min | 62.9°, 1 min | 72°, 1 min 40 sec | 30 | 623 bp |
| t-TGase | 94°, 1 min | 66.5°, 1 min | 72°, 1 min 40 sec | 33 | 1600 bp |

TABLE IIIc
Primer Sequences

| ID | Gene | | | | |
|---|---|---|---|---|---|
| No. | Name | Upstream (Sense, 5′-3′) | | Downstream (Antisense, 5′-3′) | |
| 1 | SAA | cagagttgctgctatgtccacca | (SEQ ID NO.:25) | cactccttgtgtgctcctcacc | (SEQ ID NO.:26) |
| 2 | CC3 | cctaagaggatattgacattagacagg | (SEQ ID NO.:21) | agggggaggtgggttagtag | (SEQ ID NO.:22) |
| 3 | CTGF | gcctcttcagctacctacttcctaa | (SEQ ID NO.:31) | cgaggaggaccacgaagg | (SEQ ID NO.:32) |
| 4 | Integrin | gattggtcttgccctcaacag | (SEQ ID NO.:33) | ccagcacagtcgcccaga | (SEQ ID NO.:34) |
| | B3 | | | | |
| 5 | Activin | tgattccaatgtttttctaaaagg | (SEQ ID NO.:35) | gaatgtctaaagagctcagaagt | (SEQ ID NO.:36) |
| 6 | NK4 | tggagctagaagagcccgtagg | (SEQ ID NO.:23) | gccaaaagttcaaggagccaag | (SEQ ID NO.:24) |
| 7 | Prosa- | ggtttaagcaatttctggcctct | (SEQ ID NO.:37) | cgtctgactctccgcagtctgcaat | (SEQ ID NO.:38) |
| | posin | | | | |
| 8 | Mac2-BP | gtaaaactccctgatgattccttct | (SEQ ID NO.:39) | ctctgcagactggtcctttgac | (SEQ ID NO.:40) |
| 9 | GAL-3 | tgtcttcacaaggtggaagtgg | (SEQ ID NO.:41) | ctggagggcagagcacag | (SEQ ID NO.:42) |
| 10 | MnSOD | taccaaccctaggggtaaaaataaa | (SEQ ID NO.:43) | atgctgctagtgctggtgctac | (SEQ ID NO.:44) |
| 11 | Granulin | gagactaggaagccacttctctttc | (SEQ ID NO.:45) | ctggaatgctgtgttcttttctact | (SEQ ID NO.:46) |
| 12 | p66shc | gtggcagacagggcactc | (SEQ ID NO.:47) | ctcctgagctgcctcaatg | (SEQ ID NO.:48) |
| 14 | Cathep- | ctcccgagtagctgggatta | (SEQ ID NO.:51) | ccacgtgaccaccgcgca | (SEQ ID NO.:52) |
| | sin B | | | | |
| 15 | βAPP | ttgctcctttggttcgttct | (SEQ ID NO.:27) | gctgccgaggaaactgac | (SEQ ID NO.:28) |
| 16 | t-TGase | cccagggagaaatatccactgaagcaac | (SEQ ID NO.:29) | tcgggcgggggcggtggctccttccact | (SEQ ID NO.:30) |
| 17 | Clus- | agccccttgacttctctcct | (SEQ ID NO.:53) | ctcctggcgacgccgcgtt | (SEQ ID NO.:54) |
| | terin | | | | |
| 18 | PSF | aaagtgctgggattagaggcgtga | (SEQ ID NO.:55) | tatgtattgctaagggaagctattggag | (SEQ ID NO.:56) |
| 19 | VEGF-C | gttcttggatcatcaggcaactt | (SEQ ID NO.:57) | gtggaaggaccgggggtgg | (SEQ ID NO.:58) |
| 20 | TIMP-1 | agaaccggtacccatctcaga | (SEQ ID NO.:59) | ctgtacctctggtgtctctct | (SEQ ID NO.:60) |

### EXAMPLE 9

### Production of Cells Stably Transfected with a p21-inducible Reporter Construct

To develop a stably transfected cell line with p21-regulated luciferase expression, the NK4 promoter-luciferase construct, described in Example 8 and termed pLuNK4, was introduced into HT1080 p21-9 cells, which carry IPTG-inducible p21, by cotransfection with pBabePuro carrying puromycin N-acetyltransferase as a selectable marker. Transfection was carried out using LIPOFECTAMTNE 2000 (Life Technologies, Inc., Gaithersburg, Md.), using a 10:1 ratio of pLuNK4 and pBabePuro. Stable transfectants were selected using 1 μg/mL puromycin for 5 days. 54 puromycin-resistant cell lines were isolated and tested for luciferase activity (using a Luciferase Assay System, Promega), in the presence and in the absence of 50 μM IPTG.

This assay was performed as follows. Cells were plated at a density of 40,000 cells/well in 12 well plates in 1 mL of media containing penicillin/streptomycin, glutamine and 10% fetal calf serum (FCS). After attachment, cells were treated with 50 μM IPTG or left untreated for different periods of time. Luciferase activity was then measured as described in Example 8 above. An additional aliquot was removed from the cell lysate to measure protein concentration using the Bio-Rad protein assay kit (Bradford assay). Luciferase activity for each sample was normalized to protein content and expressed as luciferase activity/μg protein. All assays were carried out in triplicate and displayed as a mean and standard deviation.

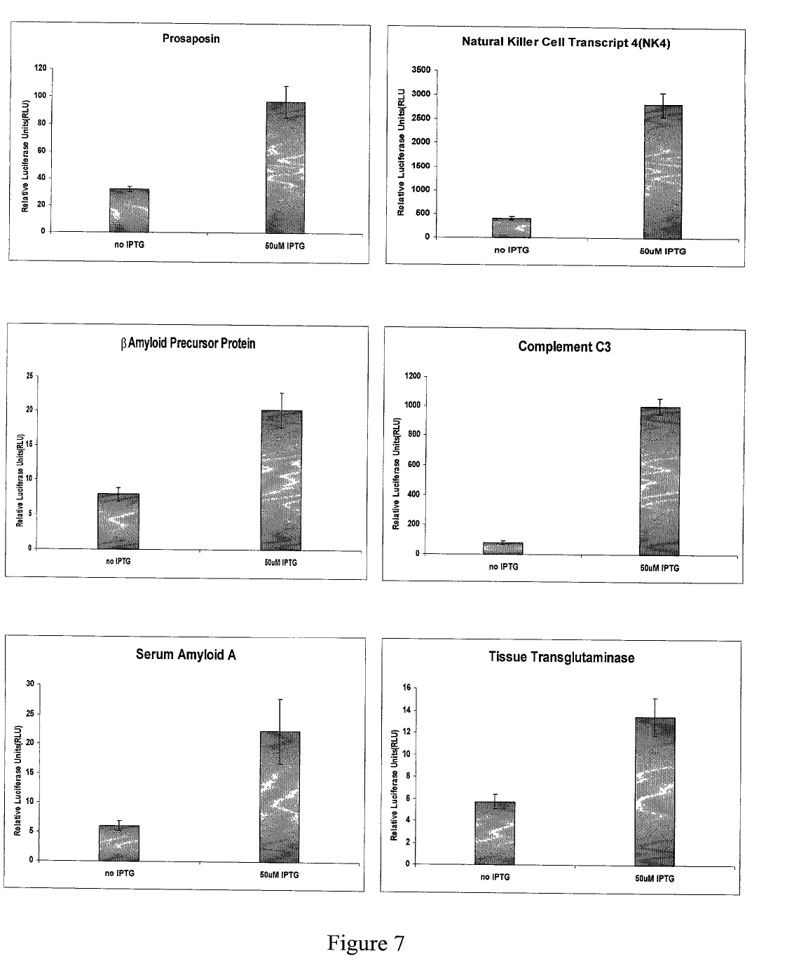

21 of 54 tested cell lines showed measurable luciferase activity, but only one cell line, designated HT1080 LuNK4p21, showed higher luciferase expression in the presence than in the absence of IPTG. The results of assays carried out with p21LuNK4 cell line are shown in FIG. 8A and 8B. FIG. 8A shows the IPTG dose dependence of luciferase expression after 24 hrs of IPTG treatment, and FIG. 8B shows the time course of luciferase expression upon the addition of 50 μM IPTG. This analysis shows that most of the induction can be achieved using as little as 5 μM IPTG and a treatment period as short as 17 hrs.

These results demonstrated that the pLuNK4 reporter construct could be used to produce stably transfected cell lines that were responsive to p21 induction of reporter gene transcription. Such constructs and cells provide a basis for a screening assay for identifying compounds that inhibit p21 -mediated gene activation. The relatively short time required for luciferase induction (about 17 hrs), together with the pronounced (approximately 3-fold) increase in luciferase levels in IPTG-treated cells, should make the LuNK4p21 cell line suitable for high-throughput screening of compounds that would inhibit the inducing effect of p21. Other cell lines with similar (and potentially better) inducibility can also be developed through the methods disclosed herein used to derive LuNK4p21. The results described in Example 8 demonstrate that the same type of screening can also be conducted using transient transfection assays with promoter constructs of p21-inducible genes rather than stably-transfected cell lines. The methods for high-throughput screening based on luciferase expression are well known in the art (see Storz et al., 1999, Analyt. Biochem. 276: 97-104 for a recent example of a transient transfection-based assay and Roos et al., 2000, Virology 273: 307-315 for an example of screening based on a stably transfected cell line). Compounds identified using these cells and assays are in turn useful for developing therapeutic agents that can inhibit or prevent p21 -mediated induction of age-related genes.

### EXAMPLE 10

Use of NFκB and p300/CBP Inhibitors to Inhibit p21-Mediated Induction in Transient Transfection Assays

Examination of promoter sequences of p21 -inducible genes showed that many of these promoters, including NK4, contain known or potential NFκB binding sites. Several p21-induced genes are known to be positively regulated by NFκB, including superoxide dismutase 2 (SOD2) (Jones et al., 1997, Mol. Cell. Biol. 17: 6970-6981), t-TGase (Mirza et al., 1997, Amer. J. Physiol. 272: G281-G288), Alzheimer's β-amyloid precursor protein (APP) (Grilli et al., 1996, J. Biol. Chem. 271: 15002-15007) and the inflammatory protein serum amyloid A (SAA) (Jensen and whitehead, 1998, Biochem J. 334: 489-503). p21 has been previously shown by transient co-transfection experiments to activate NFκB-dependent transcription (Perkins et al., 1997, Science 275: 523-527). This effect of p21 was shown to be due to the stimulation of transcription cofactors p300 and CBP (Perkins et al., 1997, Science 275: 523-527); it is possible that activation of p300/CBP or related transcription cofactors may be responsible for the effect of p21 on some of the upregulated genes. Thus, inhibitors of NFκB or p300/CBP may potentially prevent the induction of transcription by p21.

To determine if IPTG-inducible p21 expression in HT 1080 p21-9 cells stimulates the transcriptional activity of NFκB, we have used transient transfection assays to investigate the effect of p21 induction on luciferase expression from the plasmid pNFkB-Luc, commercially available from Stratagene. This plasmid expresses firefly luciferase from an artificial promoter containing five NFκB consensus sequences. To evaluate the effects of genetic inhibitors of NFκB on luciferase expression from pNFkB-Luc, 20 μg of the latter plasmid were mixed (at a molar ratio 1:2) with a plasmid MAD3 (a.k.a. pRC/βactin-HA-IKKα) that expresses a dominant mutant of IκB kinase α that selectively inhibits NFκB (DiDonato et al., 1996, Mol. Cell. Biol. 16: 1295-1304) (provided by Dr. M. Karin, University of California San Diego). This plasmid is referred to below as IKK. To determine the effect of p300/CBP inhibition on luciferase expression from pNFkB-Luc, the latter plasmid was similarly mixed in another assay with a vector expressing a truncated gene for adenoviral E1A protein with a C-terminal deletion {ΔCR2 (120-140)}. The C-truncated E1A (termed E1AΔC) is known to inhibit p300/CBP and related factors (such as PCAF) but it does not inhibit Rb, the target of the C-terminal domain of E1A (Chakravarti et al., 1999, Cell 96: 393-403). As a negative control, pNFkB-Luc was mixed with a functionally inactive form of E1A with deletions at both the C-terminus and the N-terminus {ΔN(2-36)}, termed E1AΔN/C. The E1AΔC and E1AΔN/C constructs were provided by Dr. V. Ogryzko (NICHHD, NIH). The mixtures of pNFkB-Luc with IKK, E1AΔC or E1AΔN/C were transfected into HT1080 p21 -9 cells by electroporation, as described in Example 8. After electroporation, equal numbers of transfected cells were treated with 50 μM IPTG or untreated for three days (in triplicates). The luciferase activity was measured and normalized to protein content in each sample.

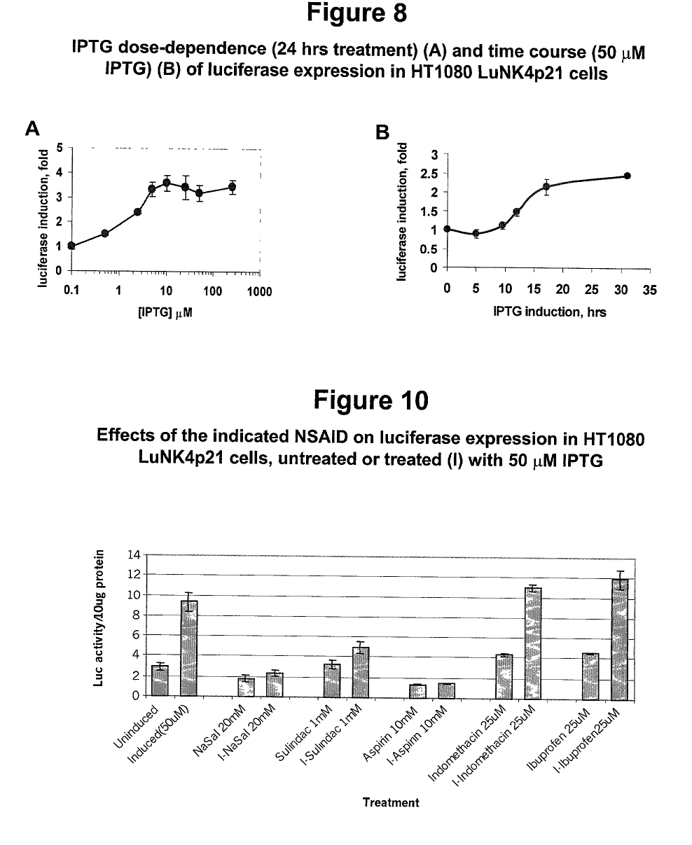

The results of this analysis are shown in FIG. 9A. pNFkB-Luc mixed with the negative control (E1AΔN/C) showed up to 15-fold induction in the presence of IPTG, demonstrating an increase in NFκB transcriptional activity in HT1080 p21-9 cells. Mixing pNFkB-Luc with the IKK inhibitor almost completely abolished luciferase expression in IPTG-treated or untreated cells, demonstrating the efficacy of this inhibitor. E1AΔC had a similar but slightly weaker effect than IKK, demonstrating the efficacy of this p300/CBP inhibitor and the requirement of p300/CBP for NFκB activity in HT1080 p21-9 cells (FIG. 9A).

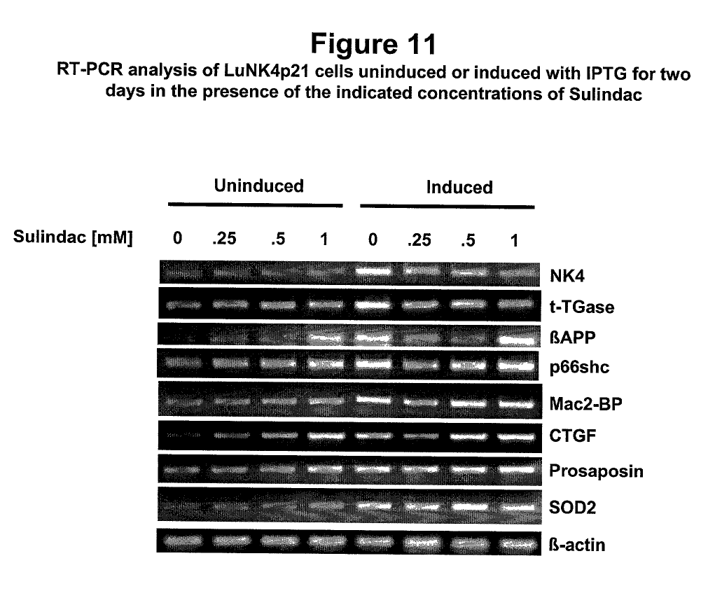

The same analysis was carried out using promoter-luciferase constructs for two p21 -inducible genes, SAA and prosaposin. The results for SAA are shown in FIG. 9B and for prosaposin in FIG. 11C. The effects of IKK and E1AΔC on the SAA promoter were almost identical to those observed with pNFkB-Luc, indicating that the regulation of this promoter by p21 is likely to be mediated through p300/CBP and NFκB (FIG. 9B). This result agrees with the demonstrated role of NFκB in SAA transcription (Jensen and Whitehead, 1998, Biochem J. 334: 489-503). Somewhat different results were obtained with the prosaposin promoter (FIG. 9C). In this experiment, E1AΔC inhibited the promoter function as strongly as it did with SAA (FIG. 9B), but IKK provided only partial inhibition of p21-induced activation of the prosaposin promoter (FIG. 9C). This result suggests that p300/CBP is essential for prosaposin transcription and its induction by p21, the effect of p300/CBP is mediated at least in part by some factors other than NFκB.

### EXAMPLE 11

### Use of Non-Steroidal Anti-Inflammatory Drugs to Inhibit p21-Mediated Gene Induction

The best-studied NFκB inhibitors in clinical use are certain non-steroidal anti-inflammatory drugs (NSAID), such as aspirin, sodium salicylate and sulindac (Kopp and Ghosh, 1994, Science 265: 956-959; Yin et al., 1998, Nature 396: 77-80; Yamamoto et al., 1999, J. Biol. Chem. 274: 27307-27314). The LuNK4p21 cell line described in Example 9 above was used to determine whether the induction of luciferase expression by p21 in this cell line can be inhibited by NSAID with NFκB -inhibitory activity.

Luciferase assays were performed substantially as described in Example 9. Luciferase activity was measured after 16 hrs of incubation with or without 50 μM IPTG, followed by an additional 20 hr treatment in the presence or in the absence of 20 mM sodium salicylate, 1 mM sulindac, or 10 mM aspirin. In addition, two NSAIDs were tested that do not inhibit NFκB: indomethacin and ibuprofen (at 25 μM each) (Yamamoto et al., 1999, ibid.). NSAID concentrations were based on the pharmacologic concentrations of these agents in the serum of patients required for their anti-inflammatory properties (Yin et al., 1998, ibid.).

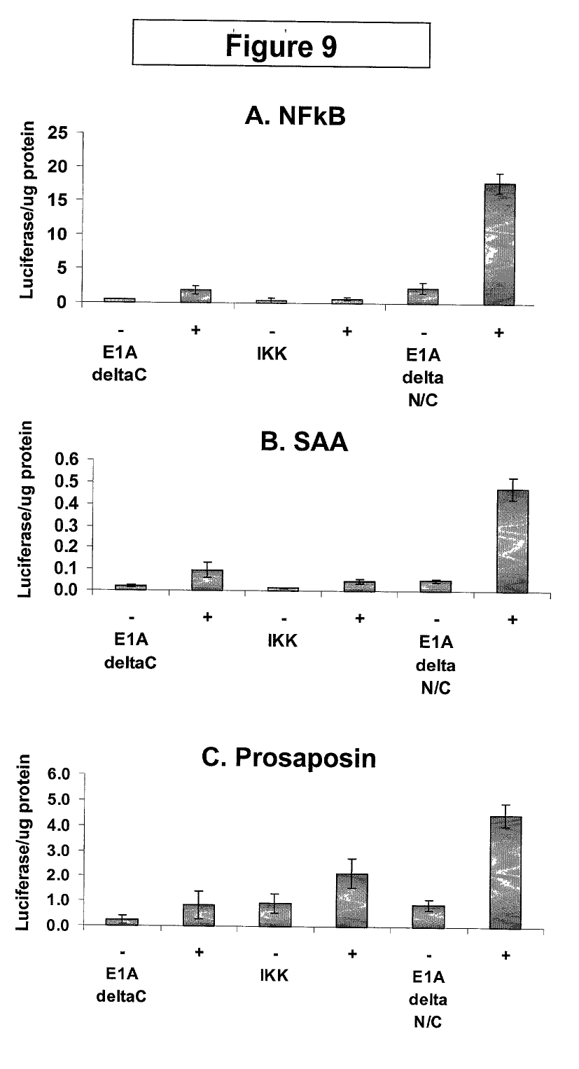

The results of these assays are shown in FIG. 10. IPTG increased luciferase expression approximately 3-4 fold in the absence of NSAID, but this induction was completely or almost completely abolished in the presence of salicylate, sulindac, or aspirin. In contrast, indomethacin and ibuprofen made no significant difference to the induction of luciferase by IPTG.

To determine whether NFκB-inhibiting NSAID inhibited not only the induction of transcription from the NK4 promoter but also RNA expression of the endogenous p21-inducible genes, LuNK4p21 cells were plated at 125,000 cells per well in 6-well plates and were either untreated or treated with 50 μM IPTG for 48 hrs (the period of time required for maximal stimulation of p21 -inducible genes; Chang et al., 2000, Proc. Natl. Acad. Sci. USA 97: 4291-4296), in the presence or in the absence of sulindac, at 250 μM, 500 μM or 1 mM concentrations. After this incubation, RNA was extracted from the cells using Qiagen RNeasy Mini Kit, and relative RNA levels of several p21-inducible genes were determined by reverse transcription-PCR (RT-PCR), essentially as described by Noonan et al. (1990, Proc. Natl. Acad. Sci. USA 87: 7160-7164), except that β-actin rather than β₂-microglobulin was used for cDNA normalization. The sequences of the PCR primers for each of the tested genes are provided in Table IVa. The PCR cycles were as follows: for the 1st cycle, 3 min for denaturation, 2 min for annealing and 2 min for extension, and the rest of cycles, 30 sec for denaturation; 30 sec for annealing; and 1 min for extension. The temperature conditions of the PCR cycles and the sizes of the PCR products are provided in Table IVb.

TABLE IVa
Primer sequences

| GENE | SENSE PRIMER (5′→3′) | ANTISENSE PRIMER (5′→3′) |
|---|---|---|
| NK4 | AGCACCAGGCCATAGAAAGA (SEQ ID NO.:13) | GGTGTCAGCTCCTCCTTGTC (SEQ ID NO.:49) |
| T-TGASE | ACTACAACTCGGCCCATGAC (SEQ ID NO.:50) | GCCAGTTTGTTCAGGTGGTT (SEQ ID NO.:61) |
| BAPP | CTCGTTCCTGACAAGTGCAA (SEQ ID NO.:62) | TGTTCAGAGCACACCTCTCG (SEQ ID NO.:63) |
| P66^{SHC} | GAGGGTGTGGTTCGGACTAA (SEQ ID NO.:64) | GCCCAGAGGTGTGATTTGTT (SEQ ID NO.:65) |
| CTGF | GGAGAGTCCTTCCAGAGCAG (SEQ ID NO.:66) | ATGTCTTCATGCTGGTGCAG (SEQ ID NO.:67) |
| MAC2-BP | ACCATGAGTGTGGATGCTGA (SEQ ID NO.:68) | ACAGGGACAGGTTGAACTGC (SEQ ID NO.:69) |
| GRANULIN | ACCACGGACCTCCTCACTAA (SEQ ID NO.:70) | ACACTGCCCCTCAGCTACAC (SEQ ID NO.:71) |
| PROSAPOSIN | CCAGAGCTGGACATGACTGA (SEQ ID NO.:72) | GTCACCTCCTTCACCAGGAA (SEQ ID NO.:73) |
| SOD2 | CAAATTGCTGCTTGTCCAAA (SEQ ID NO.:74) | CATCCCTACAAGTCCCCAAA (SEQ ID NO.:75) |
| B-ACTIN | GGGAAATCGTGCGTGACATTAAG (SEQ ID NO.:76) | TGTGTTGGCGTACAGGTCTTTG (SEQ ID NO.:77) |

TABLE IVb
PCR temperatures (in ° C.)

| Gene | Denaturation | Annealing | Extension | Cycles | Product size |
|---|---|---|---|---|---|
| NK4 | 94 | 58 | 72 | 24 | 481 |
| t-TGase | 94 | 58 | 72 | 24 | 499 |
| B-APP | 94 | 58 | 72 | 20 | 500 |
| p66^{shc} | 94 | 58 | 72 | 22 | 514 |
| CTGF | 94 | 64 | 72 | 28 | 499 |
| MAC2-BP | 94 | 58 | 72 | 21 | 517 |
| Granulin | 94 | 64 | 72 | 25 | 446 |
| Prosaposin | 94 | 58 | 72 | 21 | 500 |
| SOD2 | 94 | 58 | 72 | 23 | 505 |
| β-actin | 94 | 60 | 72 | 17 | 275 |

The results of the RT-PCR analysis are shown in FIG. 11. For NK4 (the promoter of which was used to drive luciferase expression in LuNK4p21 cells), the addition of sulindac had very little effect on gene expression in the absence of IPTG, but all the concentrations of sulindac produced a dose-dependent decrease in NK4 RNA levels in the presence of IPTG. Very similar results were obtained with t-TGase RNA. With all the other tested genes, sulindac produced a dose-dependent increase in gene expression in the absence of IPTG. As a result of this effect, the highest tested dose of sulindac (1 mM) did not decrease gene expression in the presence of IPTG, but a noticeable decrease in the IPTG effects was observed at lower doses of sulindac. In particular, the effects of IPTG were diminished by 250 and 500 μM sulindac for the APP gene, but only by 250 μM sulindac for p₆₆^{Shc}, CTGF and Mac2-binding protein (Mac2-BP) genes. None of the tested sulindac concentrations produced a significant decrease in IPTG-induced RNA levels of prosaposin or superoxide dismutase 2 (SOD2). The lack of sulindac effect on prosaposin is in agreement with a moderate effect of IKK inhibitor on the prosaposin promoter (see Example 10 above). Hence, a moderate dose of sulindac (250 μM) inhibits the ability of p21 to induce transcription for most of the tested genes.

These results demonstrated that assays for interference with p21-mediated induction of reporter expression from the promoters of p21 -inducible genes are capable of identifying agents that inhibit p21-mediated induction of genes associated with carcinogenesis and age-related diseases. In particular, an agent (sulindac) that was first identified as an effective inhibitor in a promoter-based assay using LuNK4p21 cell line was found to inhibit the induction of several aging-associated genes by p21. These results further demonstrated that NSAIDs that are active as NFκB inhibitors can prevent the induction of aging-associated genes by CDK inhibitors.

Agents that inhibit the induction of transcription by CDK inhibitors may be clinically useful for chemoprevention or slowing down the development of age-related diseases, including Alzheimer's disease, amyloidosis, atherosclerosis and arthritis. In addition, such compounds, through their effects on the expression of secreted growth factors (such as CTGF) may have value in cancer therapy or prevention. In fact, the available clinical data on NSAIDs with NFκB -inhibitory activity support these fields of use. Thus, several NSAID, including sulindac, aspirin and salicylate, were shown to have chemopreventive value in colorectal carcinomas and various other types of cancer and promoted the disappearance of colonic polyps (Lee et al., 1997, “Use of aspirin and other nonsteroidal anti-inflammatory drugs and the risk of cancer development.” in DeVita et al., eds., CANCER. PRINCIPLES & PRACTICE OF ONCOLOGY, Lippincott-Raven: Philadelphia, pp. 599-607). The use of aspirin and other NSAIDs was also shown to decrease the risk of Alzheimer's disease (Stewart etal., 1997, Neurology 48: 626-632). Long-term aspirin therapy was further reported to decrease the incidence of atherosclerosis (Sloop, 1998, Angiology 49: 827-832). Finally, sulindac has been one of the most commonly used drugs with proven clinical efficacy in the treatment of arthritis (Brogden et al., 1978, Drugs 16: 97-114). While some of these beneficial effects of NSAIDs have been attributed to their activity as cyclooxygenase 2 inhibitors (Pennisi, 1998, Science 280: 1191-1192), the results disclosed herein suggest that these clinical activities may also be due to the inhibition of p21-induced gene expression, presumably through the NFκB -inhibitory activity of these compounds. The assays and screening system provided by the instant invention enable one with ordinary skill in the art to test various NSAID derivatives for the improvement in this activity. Furthermore, these results provide the basis for using the general category of NFκB and p300/CBP inhibitors as agents for chemoprevention or treatment of cancer and age-related diseases.

It should be understood that the foregoing disclosure emphasizes certain specific embodiments of the invention and that all modifications or alternatives equivalent thereto are within the spirit and scope of the invention as set forth in the appended claims.

TABLE I
Genes downregulated by p21 induction

| | | Balanced Diff. | Confirmed |
|---|---|---|---|
| Genes | Accession No. | Expr. | by^{a} |
| A. p21-inhibited genes identified by UniGem V array: | | | |
| Associated with mitosis: | | | |
| CDC2 | X05360 | 2.5 | R, W |
| CKsHs1 (CDC2 kinase) | X54941 | 5.5 | R |
| PLK1 (polo-like kinase) | U01038 | 5.1 | R, W |
| XCAP-H condensin homolog | D38553 | 6 | R |
| CENP-A (centromere protein A) | U14518 | 5.3 | R |
| CENP-F (centromere protein F) | U30872 | 2.5 | R |
| MAD2 | U65410 | 6.6 | R, W |
| BUBR1 | AF053306 | 5.9 | R |
| MCAK (mitotic centromere-associated kinesin) | U63743 | 3.8 | R |
| HSET kinesin-like protein | AL021366 | 3.6 | R |
| CHL1 helicase | U75968 | 3.3 | R |
| AIK-1 (aurora/IPL1-related kinase) | D84212 | 4.6 | R |
| AIM-1 (AIK-2; aurora/IPL1-related kinase) | AF004022 | 10.2 | R |
| PRC1 (protein regulating cytokinesis 1) | AF044588 | 12 6 | R, W |
| Citron kinase | H10809 | 2.7 | R |
| Lamin B1 | L37747 | 7 | |
| Lamin B2 | M94362 | 2.7 | |
| LAP-2 (lamin-associated protein 2) | U18271 | 4 6 | R |
| MPP2 (M phase phosphoprotein 2) | U74612 | 3.7 | R |
| MPP5 (M phase phosphoprotein 5) | X98261 | 3.7 | |
| Associated with DNA replication, segregation and | | | |
| chromatin assembly: | | | |
| Thymidine kinase 1 | K02581 | 2.9 | R |
| Thymidylate synthase | X02308 | 3.9 | R |
| Uridine phosphorylase | X90858 | 2.5 | |
| Ribonucleotide reductase M1 | X59543 | 4.6 | R |
| Ribonucleotide reductase M2 | X59618 | 10.7 | R |
| CDC47 homolog (MCM7) | D55716 | 9.6 | R |
| CDC21 homolog (MCM4) | X74794 | 2.7 | R |
| CDC45 homolog (Porc-PI) | AJ223728 | 4.1 | R |
| HsORC1 (origin recognition complex 1) | U40152 | 2.7 | R |
| DNA polymerase α | X06745 | 2.8 | R |
| Replication factor C (37-kD subunit) | M87339 | 2.6 | |
| B-MYB | X13293 | 9.1 | |
| HPV16 E1 protein binding protein | U96131 | 3.7 | |
| Topoisomerase IIα | J04088 | 8.6 | R |
| Chromatin assembly factor-I (p60 subunit) | U20980 | 2.7 | R |
| High-mobility group chromosomal protein 2 | X62534 | 3.7 | R |
| High-mobility group chromosomal protein 1 | D63874 | 3.6 | R |
| Histone H2A.F/Z variant | AA203494 | 2.8 | |
| Associated with DNA repair: | | | |
| XRCC9 | U70310 | 3 6 | |
| RAD54 homolog | X97795 | 5.4 | R |
| HEX1 5′-3′ exonuclease (RAD2 homolog) | AF042282 | 5.2 | R |
| ATP-dependent DNA ligase I | M36067 | 2 5 | R |
| RAD21 homolog | D38551 | 2.9 | R |
| Associated with transcription and RNA processing: | | | |
| Putative transcription factor CA150 | AF017789 | 2.8 | |
| Transcriptional coactivator ALY | AF047002 | 3.3 | |
| WHSC1/MMSET (SET domain protein) | AA401245 | 2.9 | |
| NN8-4AG (SET domain protein) | U50383 | 2.8 | |
| EZH2 (enhancer of zeste homolog 2) | U61145 | 2.8 | |
| PTB-associated splicing factor | X70944 | 2.5 | |
| AU-rich element RNA-binding protein AUF1 | U02019 | 2.8 | |
| U-snRNP-associated cyclophilin | AF016371 | 2.8 | |
| Other genes: | | | |
| 3-phosphoglycerate dehydrogenase | AF006043 | 4.8 | |
| L-type amino acid transporter, subunit LAT1 | M80244 | 4.1 | R |
| Hyaluronan-mediated motility receptor | U29343 | 4 | |
| Phorbolin I (PKC-inducible) | U03891 | 3.9 | |
| PSD-95 binding family protein | D13633 | 3.7 | R |
| HTRIP (TNF receptor component) | U77845 | 3.6 | |
| NAD-dependent methylenetetrahydrofolate dehydrogenase | X16396 | 3.4 | |
| Membrane glycoprotein 4F2 antigen heavy chain | J02939 | 3.2 | |
| Mucin-like protein | D79992 | 3.2 | |
| MAC30 (differentially expressed in meningiomas) | L19183 | 2.9 | |
| P52rIPK (regulator of interferon-induced protein kinase) | AF007393 | 2.8 | |
| Putative phosphoserine aminotransferase | AA192483 | 2.8 | |
| Glucose 6-phosphate translocase | Y15409 | 2.7 | |
| Calcyclin binding protein | AF057356 | 2.6 | |
| Ornithine decarboxylase 1 | X16277 | 2.6 | R |
| Trophinin assisting protein (tastin) | U04810 | 2.5 | |
| Acyl-coenzyme A cholesterol acyltransferase | L21934 | 2.5 | |
| Pinin/SDK3 | Y10351 | 2.5 | |
| Genes with unknown function: | | | |
| EST | AA975298 | 2.7 | |
| EST | AA034414 | 2.5 | |
| EST | AA482549 | 2.5 | |

| Genes | Accession No. | UniGemV result^{b} |
|---|---|---|
| B. p21-inhibited genes identified by RT-PCR: | | |
| Cyclin A1 | U66838 | IS |
| Cyclin B1 | M25753 | IS |
| CDC25A | NM_001789 | A |
| Dihydrofolate reductase | J00140 | 1.5 |
| ING1 | NM_005537 | A |

^{a}Abbreviations: R, RT-PCR; W, western blotting
^{b}Abbreviations: IS, insufficient signal; A, absent from the array

TABLE II
Genes upregulated by p21 induction

| | | | |
|---|---|---|---|
| | Accession | Balanced Diff | |
| Genes | No | Expr | Confirmed by^{a} |

Secreted proteins and proteins associated with extracellular matrix:

| | | | |
|---|---|---|---|
| Fibronectin 1 | X02761 | 5.7 | R |
| Plasminogen activator inhibitor, type I | M14083 | 3.7 | R, N |
| Plasminogen activator, tissue type | M15518 | 2.8 | Z |
| Laminin β2 | X79683 | 2.1 | |
| Desmocollin 2a/bb | X56807 | 3.5 | |
| Podocalyxin-like protein | U97519 | 2 | |
| Activin A (inhibin βA) | J03634 | 2 | R |
| Galectin 3 (Mac-2) | AB006780 | 2.4 | N |
| Mac-2 binding protein | L13210 | 2 | R, N |
| Prosaposin | J03077 | 2.9 | N |
| CTGF (connective tissue growth factor) | M92934 | 3.3 | N |
| Granulin/epithelin | AF055008 | 2.1 | N |
| Cathepsin B | L04288 | 2.4 | N |
| Tissue transglutaminase | M55153 | 2.5 | R, N, W |
| P37NB (slit homolog) | U32907 | 2.1 | |
| Serum amyloid A protein precursor | M26152 | 4 | R, N, W |
| Alzheimer's disease amyloid A4 protein precursor | D87675 | 2 | R, N |
| Complement C3 precursor | K02765 | 5.9 | R, N |
| Testican | X73608 | 2.1 | N |
| Integrin β3 | M35999 | 2.1 | R, N |
| Lysosomal proteins: | | | |
| N-acetylgalactosamine-6-sulfate sulfatase | U06088 | 2.3 | N |
| Acid alpha-glucosidase | X55079 | 2.4 | N |
| Acid lipase A (cholesterol esterase) | X76488 | 2.1 | N |
| Lysosomal pepstatin-insensitive protease (CLN2) | AF017456 | 2.5 | |
| Mitochondrial proteins: | | | |
| Superoxide dismutase 2 | X07834 | 3.5 | R, N, W |
| Metaxin | J03060 | 3.4 | |
| 2,4-dienoyl-CoA reductase | U78302 | 2 | |
| Other genes associated with stress response and | | | |
| signal transduction: | | | |
| Ubiquitin-conjugating enzyme (UbcH8) | AF031141 | 2 | |
| Ubiquitin-specific protease 8 | D29956 | 2 | |
| RTP/Cap43/Drg1/Ndr1 (Inducible by nickel, retinoids, | D87953 | 2.5 | |
| homocysteine and ER stress) | | | |
| C-193 muscle ankyrin-repeat nuclear protein (cytokine- | X83703 | 3 | |
| inducible) | | | |
| LRP major vault protein associated with multidrug resistance | X79882 | 2.2 | N |
| β-arrestin related HHCPA78 homolog (upregulated by | S73591 | 4.1 | N |
| vitamin D3) | | | |
| R-RAS | M14949 | 2.4 | |
| RAB 13 small GTPase | X75593 | 2.2 | |
| P66 SHC (ski oncogene) | U73377 | 2 | N |
| MK-STYX (MAP kinase phosphatase-like protein) | N75168 | 2 | |
| H73 nuclear antigen/MA-3 apoptosis-related/TIS | U96628 | 2.4 | |
| (topoisomerase-inhibitor suppressed) | | | |
| Other genes: | | | |
| Natural killer cells protein 4 | M59807 | 4.4 | R |
| TXK tyrosine kinase (T-cell specific) | L27071 | 3.8 | |
| X-linked PEST-containing transporter | U05321 | 2.1 | |
| AMP deaminase 2 | M91029 | 2 | N |
| FIP2/HYPL huntingtin-interacting protein | AF061034 | 2 | |
| DNASE I homolog | X90392 | 2.5 | N |
| Transcription factor 11 | X77366 | 2 | |
| Histone H2A.2 | L19779 | 2.8 | |
| Histone H2B | AL021807 | 2.4 | |
| Genes with unknown function: | | | |
| 23808 | AF038192 | 2.1 | |
| CGI-147 | AA307912 | 2.1 | N |
| EST | W89120 | 2.8 | |
| EST | AI026140 | 2.5 | |
| EST | AA218982 | 2.4 | |
| EST | W63684 | 2 | |

^{a}Abbreviations: R, RT-PCR; N, northern hybridization; W, western blotting; Z, zymography

We claim:

1. A recombinant expression construct encoding a reporter gene operably linked to a promoter from a mammalian gene induced by a cyclin-dependent kinase inhibitor.

2. A recombinant expression construct according to claim 1, wherein the reporter gene encodes firefly luciferase, Renilla luciferase, chloramphenicol acetyltransferase, beta-galactosidase, green fluorescent protein, or alkaline phosphatase.

3. A recombinant expression construct according to claim 1, wherein the promoter is a promoter from a human gene induced by a CDK inhibitor.

4. A recombinant expression construct according to claim 3, wherein the promoter is a promoter from a human gene identified in Table II

5. A recombinant expression construct according to claim 4, wherein the promoter is a promoter from a serum amyloid A (SEQ ID NO.: 1), complement C3 (SEQ ID NO.: 2), connective tissue growth factor (SEQ ID NO.: 3), integrin β-3 (SEQ ID NO.: 4), activin A (SEQ ID NO.: 5), natural killer cell protein 4 (SEQ ID NO.: 6), prosaposin (SEQ ID NO.: 7), Mac2 binding protein (SEQ ID NO.: 8), galectin-3 (SEQ ID NO.: 9), superoxide dismutase 2 (SEQ ID NO.: 10), granulin/epithelin (SEQ ID NO.: 11), p66^{shc} (SEQ ID NO.: 12), cathepsin B (SEQ ID NO.: 14), β-amyloid precursor protein (SEQ ID NO.: 15), tissue transglutaminase (t-TGase; SEQ ID NO.: 16), clusterin (SEQ ID NO. 17), pro stacyclin stimulating factor (EQ ID NO.: 18), vascular endothelial growth factor-C (SEQ ID NO. 19) and tissue inhibitor of metalloproteinases-1 (SEQ ID NO. 20).

6. A recombinant expression construct according to claim 4, wherein the promoter is apromoter from human natural killer cell protein 4 (SEQ ID NO.: 6), serum amyloid A (SEQ ID NO.: 1), complement C3 (SEQ ID NO.: 2), tissue transglutaminase (SEQ ID NO.: 16), β-amyloid precursor protein (SEQ ID NO.: 15), or prosaposin (SEQ ID NO.: 7).

7. A recombinant expression construct according to claim 4, wherein the recombinant expression construct is pLuNK4.

8. A mammalian cell comprising a recombinant expression construct according to claim 1, 2, 3, 4, 5 or 6.

9. The mammalian cell of claim 8, identified by A.T.C.C. Accession No. PTA 3381 (HT1080 LuNK4p21).

10. A mammalian cell according to claim 8 wherein expression of the recombinant expression construct is modulated by NFkB.

11. A mammalian cell according to claim 8, further comprising a second recombinant expression construct encoding a mammalian CDK inhibitor gene.

12. A mammalian cell according to claim 11, wherein expression of the CDK inhibitor is experimentally-induced in the mammalian cell.

13. The mammalian cell of claim 11, wherein the recombinant expression construct encoding a mammalian CDK inhibitor gene is under the transcriptional control of an inducible promoter, wherein expression of the CDK inhibitor from the recombinant expression construct is mediated by contacting the recombinant cell with an inducing agent that induces transcription from the inducible promoter or by removing an agent that inhibits transcription from such a promoter.

14. The mammalian cell of claim 13, wherein the mammalian CDK inhibitor gene is a human p21 gene or CDK-binding fragment thereof.

15. The mammalian cell of claim 13, wherein the mammalian CDK inhibitor gene is a human p16 gene or CDK-binding fragment thereof.

16. A mammalian cell according to claim 13, further comprising a recombinant expression construct encoding a bacterial lactose repressor, wherein transcription thereof is controlled by a mammalian promoter, wherein the recombinant expression construct encoding a mammalian CDK inhibitor gene comprises a lactose repressor-responsive promoter element and wherein transcription of the CDK inhibitor gene is controlled by said lactose-repressor responsive promoter element, and wherein expression of the CDK inhibitor gene from the recombinant expression construct is mediated by contacting the recombinant cell with a lactose repressor-specific inducing agent.

17. The mammalian cell of claim 8, wherein the cell is a human HT1080 fibrosarcoma cell.

18. The mammalian cell of claim 11, wherein the cell is a human HT1080 fibrosarcoma cell.

19. The mammalian cell of claim 16, wherein the cell is a human HT1080 fibrosarcoma cell.

20. The mammalian cell of claim 11, wherein the second expression construct is LNp21CO3.

21. The mammalian cell of claim 20, identified by A.T.C.C. Accession No. PTA 1664 (p21-9).

22. The mammalian cell of claim 11, wherein the second expression construct is LNp16RO2.

23. The mammalian cell of claim 22, identified by A.T.C.C. Accession No. PTA-2580 (HT1080/LNp16RO2) or clonal derivatives thereof.

24. The mammalian cell of claim 16, wherein the lactose repressor-specific inducing agent is a β-galactoside.

25. A method for identifying a compound that inhibits induction of genes induced by a CDK inhibitor in a mammalian cell, the method comprising the steps of:
(a) culturing a recombinant mammalian cell according to claim 8 under conditions that induce expression of genes induced by a CDK inhibitor in mammalian cells in the presence and absence of a compound;
(b) comparing reporter gene expression in said cell in the presence of the compound with reporter gene expression in said cell in the absence of the compound; and
(c) identifying the compound that inhibits induction of genes induced by a CDK inhibitor if reporter gene expression is lower in the presence of the compound than in the absence of the compound.

26. The method of claim 25, wherein the cell is cultured under conditions that induce expression of a CDK inhibitor in said cell.

27. The method of claim 26, wherein the CDK inhibitor is p21 or p16 or CDK-binding fragments thereof.

28. The method of claim 25, wherein the cell is further comprises a second recombinant expression construct encoding a mammalian CDK inhibitor gene.

29. The method of claim 28, wherein the second recombinant expression construct comprises a mammalian CDK inhibitor gene under the transcriptional control of an inducible promoter, wherein expression of the CDK inhibitor from the recombinant expression construct is mediated by contacting the recombinant cell with an inducing agent that induces transcription from the inducible promoter orby removing an agent that inhibits transcription from such promoter.

30. The method of claim 29, wherein the mammalian CDK inhibitor gene is a human p21 gene or CDK-binding fragment thereof.

31. The method of claim 29, wherein the mammalian CDK inhibitor gene is a human p16 gene or CDK-binding fragment thereof.

32. The method of claim 29, wherein the cell is a human HT1080 fibrosarcoma cell.

33. The method of claim 29, wherein the mammalian cell further comprises a recombinant expression construct encoding a bacterial lactose repressor, wherein transcription thereof is controlled by a mammalian promoter, wherein the recombinant expression construct encoding a mammalian CDK inhibitor gene comprises a lactose repressor-responsive promoter element and wherein transcription of the CDK inhibitor gene is controlled by said lactose-repressor responsive promoter element, and wherein expression of the CDK inhibitor gene from the recombinant expression construct is mediated by contacting the recombinant cell with a lactose repressor-specific inducing agent.

34. A method for identifying a compound that inhibits CDK inhibitor-mediated induction of cellular gene expression, the method comprising the steps of:
(a) producing expression of a CDK inhibitor in a mammalian cell;
(b) assaying the cell in the presence of the compound for changes in expression of cellular genes whose expression is modulated by the CDK inhibitor; and
(c) identifying the compound as an inhibitor of CDK inhibitor-mediated modulation of cellular gene expression if expression of the cellular genes of subpart (b) is changed to a lesser extent in the presence of the compound.

35. The method of claim 34 wherein the CDK inhibitor is p16 or p21.

36. The method of claim 35, wherein the mammalian cell comprises a recombinant expression construct encoding a mammalian CDK inhibitor under the transcriptional control of an inducible heterologous promoter, wherein expression of the CDK inhibitor p21 from the recombinant expression construct is mediated by contacting the recombinant cell with an inducing agent that induces transcription from the inducible promoter or by removing an agent that inhibits transcription from such promoter.

37. The method of claim 36, wherein the CDK inhibitor is p16.

38. The method of claim 36, wherein the CDK inhibitor is p21.

39. The method of claim 34, wherein expression of the cellular gene is induced by p21.

40. The method of claim 34, wherein expression of the cellular gene is induced by p16.

41. The method of claim 39, wherein the cellular gene is identified in Table II.

42. The method of claim 40, wherein the cellular gene is identified in Table II.

43. The method of claim 34, wherein expression of the cellular gene is detected using an immunological reagent.

44. The method of claim 34, wherein expression of the cellular gene is detected by assaying for an activity of the cellular gene product.

45. The method of claim 34, where expression of the cellular gene is detected by hybridization to a complementary nucleic acid.

46. A method for identifying a compound that inhibits CDK inhibitor-mediated induction of cellular gene expression in a mammalian cell, the method comprising the steps of:
(a) treating the mammalian cell in the presence and absence of the compound with an agent or culturing the mammalian cell under conditions that induce senescence;
(b) assaying the mammalian cell for induction of genes that are induced by CDK inhibitor gene expression; and
(c) identifying the compound as an inhibitor of CDK inhibitor-mediated induction of cellular gene expression if genes that are induced by the CDK inhibitor are induced to a lesser extent, in the presence of the compound than in the absence of the compound.

47. The method of claim 46, wherein the CDK inhibitor is p21 or p16.

48. The method of claim 46, wherein the genes are identified in Table II.

49. The method of claim 46, wherein expression of the cellular gene is detected using an immunological reagent.

50. The method of claim 46, wherein expression of the cellular gene is detected by assaying for an activity of the cellular gene product.

51. The method of claim 46, where expression of the cellular gene is detected by hybridization to a complementary nucleic acid.

52. A method for identifying a compound that inhibits CDK inhibitor-mediated induction of cellular gene expression in a mammalian cell, the method comprising the steps of:
(a) contacting a mammalian cell in the presence or absence of the compound with an agent or culturing the mammalian cell under conditions that induce senescence, wherein the cell comprises a reporter gene under the transcriptional control of a promoter for a mammalian gene whose expression is modulated by a CDK inhibitor;
(b) assaying the cell for changes in expression of the reporter gene; and
(c) identifying the compound as an inhibitor of CDK inhibitor-mediated induction of cellular gene expression if expression of the reporter gene is changed to a lesser degree in the presence of the compound than in the absence of the compound.

53. The method of claim 52, wherein the CDK inhibitor is p21 or p16.

54. The method of claim 52, wherein the mammalian gene promoter is a promoter of a mammalian gene identified in Table II.

55. The method of claim 52, wherein expression of the cellular gene is detected using an immunological reagent.

56. The method of claim 52, wherein expression of the cellular gene is detected by assaying for an activity of the cellular gene product.

57. The method of claim 52, where expression of the cellular gene is detected by hybridization to a complementary nucleic acid.

58. A m ethod for inhibiting CDK inhibitor-mediated induction of cellular gene expression, the method comprising the step of contacting the cell with a compound produced according to the method of claim 25.

59. A method for inhibiting CDK inhibitor-mediated induction of cellular gene expression, the method comprising the step of contacting the cell with a compound produced according to the method of claim 34.

60. A method for inhibiting CDK inhibitor-mediated induction of cellular gene expression, the method comprising the step of contacting the cell with a compound produced according to the method of claim 46.

61. A method for inhibiting CDK inhibitor-mediated induction of cellular gene expression, the method comprising the step of contacting the cell with a compound produced according to the method of claim 52.

62. A method for inhibiting CDK inhibitor-mediated induction of cellular gene expression, the method comprising the step of contacting the cell with an effective amount of a compound that inhibits NFKB activity.

63. A method for treating a disease in an animal accompanied by CDK inhibitor induced gene expression, the method comprising the step of administering to the animal an effective amount of a non-steroidal anti-inflammatory drug (NSAID) that inhibits NFkB activity.

64. A method according to claim 63, wherein the disease is cancer other than colon cancer.

65. A method according to claim 63, wherein the disease is renal failure.

66. A method according to claim 63, wherein the disease is Alzheimer's disease and the NSAID is other than aspirin or salicylate.

67. A method according to claim 63, wherein the disease is atherosclerosis and the NSAID is other than aspirin.

68. A method according to claim 63, wherein the disease is arthritis and the NSAID is other than aspirin, sulindac or salicylate.

69. A compound that inhibits genes associated with pathogenic consequences of senescence in a mammalian cell, wherein the compound is produced by a method having the steps of:
(a) treating the mammalian cell in the presence of the compound with an agent or culturing the mammalian cell under conditions that induce senescence;
(b) assaying the mammalian cell for induction of genes that are induced by CDK inhibitor gene expression; and
(c) identifying the compound as an inhibitor of senescence if genes that are induced by the CDK inhibitor are induced to a lesser extent, in the presence of the compound.

70. A compound of claim 69, wherein the CDK inhibitor is p21 or p16.

71. A compound that inhibits production of gene products induced by a CDK inhibitor in a mammalian cell, wherein the compound is produced by a method having the steps of:
(a) treating the mammalian cell in the presence of the compound with an agent or culturing the mammalian cell under conditions that induce expression of a CDK inhibitor;
(b) assaying the mammalian cell for induction of genes that are induced by CDK inhibitor gene expression; and
(c) identifying the compound as an inhibitor of CDK inhibitor induction if genes that are induced by the CDK inhibitor are induced to a lesser extent, in the presence of the compound.

72. A compound of claim 71, wherein the CDK inhibitor is p21 or p16.

73. A method for inhibiting production of anti-apoptotic or mitogenic factors in a mammalian cell, the method comprising the steps of contacting the cell with a compound that inhibits induction of gene expression by a CDK inhibitor.

74. The method of claim 73, wherein the mammalian cell is a stromal fibroblast.

75. The method of claim 73, wherein the compound is an NFκB inhibitor or a p300/CPB inhibitor.

76. A method for treating an animal to prevent or ameliorate the effects of a disease accompanied by CDK inhibitor induced gene expression, the method comprising the steps of administering to an animal in need thereof a therapeutically-effective dose of a pharmaceutical composition of a compound identified according to the method of claims 25, 34, 46, or 52.

77. A method for inhibiting or preventing expression of a gene induced by a CDK inhibitor in a mammalian cell, the method comprising the step of contacting the mammalian cell with an amount of a compound identified according to the method of claims 25, 34, 46, or 52 effective to inhibit or prevent expression of the a gene induced by a CDK inhibitor.

78. A method for selectively inhibiting induction of genes induced by a CDK inhibitor in an animal, comprising administering an NFkB inhibitor to an animal in need of such treatment.

79. A method of claim 78, wherein the NFkB inhibitor is a non-steroidal anti-inflammatory compound.

80. The method of claim 79, wherein the animal is a human.

81. A method for selectively inhibiting induction of genes induced by a CDK inhibitor in an animal, comprising administering to the animal a compound produced by the method of claim 25.

82. The method of claim 81, wherein the animal is a human.

83. A method for selectively inhibiting induction of genes induced by a CDK inhibitor in an animal, comprising administering to the animal a compound produced by the method of claim 34.

84. The method of claim 83, wherein the animal is a human.

85. A method for selectively inhibiting induction of genes induced by a CDK inhibitor in an animal, comprising administering to the animal a compound produced by the method of claim 46.

86. The method of claim 85, wherein the animal is a human.

87. A method for selectively inhibiting induction of genes induced by a CDK inhibitor in an animal, comprising administering to the animal a compound produced by the method of claim 52.

88. The method of claim 87, wherein the animal is a human.

89. A method for selectively inhibiting induction of genes induced by a CDK inhibitor in an animal, comprising administering to the animal a compound produced by the method of claim 69.

90. The method of claim 89, wherein the animal is a human.

91. A method for selectively inhibiting induction of genes induced by a CDK inhibitor in an animal, comprising administering to the animal a compound produced by the method of claims 25, 34, 46, or 52.

92. The method of claim 91, wherein the animal is a human.

93. A method for selectively inhibiting induction of genes induced by a CDK inhibitor in an animal, comprising administering to the animal a compound according to claim 71.

94. The method of claim 93, wherein the animal is a human.


SEQUENCE LISTING (77 sequences)

SEQ ID NO: 1 - DNA, 1200 bp, Homo sapiens
  misc_feature: Human serum amyloid A (SAA) gene, 5′ flank (AN M26698)
ttgcccaggctgggcctcaaatttctgggttcaagcaggcctcctgccttggcctcccaagtagctgggacatatggcacatgccaccatgcctggcccatttctaaattgcttgtttgtttgttattacaaatgcctagcccctcagggtatgaacatggactggagaagaagaaaccagagttgctgctatgtccaccagcctctctgcatgtcctggcctcagcccccctgggctctggtactgacccatctctggccaccatgctcctccataagcctctgcagagctaatctgaccctgttgatgttctcatgagagagtgatctgaatgccccctgaacccctccgtgataatacagcagaccaagagctctcccacccttccctgcctggatgctgggcacgtccccagctgggctgcctatttaacgcaccacactctcattctcccaaggtggggctccaggactaggctggggcagcagaaagtccccctctctacattgtccttggctcaggagccaacttagaaaaagcatttccaaattggctaagccagcggagcagagattttctgtgctgagaaatatcaggacatccagaggggtggaaggaggcttccagggcacacatgagatgtggcaggggtaggctgtccgttttaaagcttaaagctttagacatgaactcacagggacttcagtcagggtcatctgccatgtggcccagcagggcccatcctgaggaaatgaccggtatagtcaggagctggctgaagagctgccctcactccacaccttccagcagcccaggtgccgccatcacggggctcccactggcatctctgcagctgcacttcccccaatgctgaggagcagagctgatctagcaccctgtccattgccaaggcacagcaaacctctcttgttcccataggttacacaactgggataaatgacccgggatgaagaaaccaccggcatccaggaacttgtcttagaccagtttgtaggggaaatgacctgcagggactttccccagggaccacatccagcttttcttccctcccaagagaccagcaaggctcactataaatagcagccacctctccctggcagacagggacccgcagctcagctacagcacagatcaggtgaggagcacacaaggagtgatttttaaaacttactctgttt

SEQ ID NO: 2 - DNA, 1018 bp, Homo sapiens
  misc_feature: Human complement C3 gene,  exon 1 (AN M63423)
gatcaatatgaatatattatacacacagacacacacacagacacacacacacacacacacacaaacaatacaatttaatatcctaagaggatattgacattagacaggtacaaaagctctagaaatgaggactttcctcagtgatgacttttttcaccaccaaagtcactcaggcatcctgacaagggtaagtgaggggagcctccttggaaaataaactcacttggatagtgaactcctgcacatacctcaaagcccatctgaaatgtcccctcctacaggaagttttccctgaccctccaagaagcagagttctatttcactggggaaaacatttcttcttcttcttttttttccctgccctgcacatgagctagaaaacatttcatgaaactgggagtttctgtgctgggctctgtccctcccccattctacttcccctccctcagcatggaagcctctggaagtggggctctgactcccagcctacagagagattcctaggaagtgttcgactgataaacgcatggccaaaagtgaactggggatgaggtccaagacatctgcggtggggggttctccagaccttagtgttcttccactacaaagtgggtccaacagagaaaggtctgtgttcaccaggtggccctgaccctgggagagtccagggcagggtgcagctgcattcatgctgctggggaacatgccctcaggttactcaccccatggacatgttggccccagggactgaaaagcttaggaaatggtattgagaaatctggggcagccccaaaaggggagaggccatggggagaagggggggctgagtgggggaaagcagagccagataaaaagccagctccagcaggcgctgctcactcctccccatcctctccctctgtccctctgtccctctgaccctgcactgtcccagcaccatgggacccacctcaggtcccagcctgctgctcctgctactaacccacctccccctggctctggggagtcccatgtgagt

SEQ ID NO: 3 - DNA, 687 bp, Homo sapiens
  misc_feature: CTGF gene and promoter region (AN X92511)
cgaattttttaggaattcctgctgtttgcctcttcagctacctacttcctaaaaaggatgtatgtcagtggacagaacagggcaaacttattcgaaaaagaaataagaaataattgccagtgtgtttataaatgatatgaatcaggagtggtgcgaagaggatagggaaaaaaaaattctatttggtgctggaaatactgcgcttttttttttcctttttttttttttctgcgagctggagtgtgccagctttttcagacggaggaatgctgagtgtcaaggggtcaggatcaatccggtgtgagttgatgaggcaggaaggtggggaggaatgcgaggaatgtccctgtttgtgtagactccattcagctcattggcgagcgcgccgcccggagcgtataaaagcctcggccgcccgccccaaactcacacaacaactcttccgctgagaggagacagccagtgcgactccaccctccagctcgacggcagccgccccggccgacagccccgagacgacagcccggccggtcccggtccccacctccgaccaccgccagcgctccaggccccgcgctccccgctcgccgccaccgcgccctcccgtccgcccgcagtgccaaccatgaccgccgccagtatgggccccgtccgcgtcgccttcgtggtcctcctcgccctctgcag

SEQ ID NO: 4 - DNA, 584 bp, Homo sapiens
  misc_feature: Intergin beta-3 subunit gene, promoter region (AN L28832)
aagcttgggatgtggtcttgccctcaacaggtaggtagtctaccggaaaaccaaactaaggcaagaaaaaaattagtgaataataaaggactgaaccggttcagagaaggcattcagcagatgtttgccagtcaaatgaattaaagtgtgaatgaatgaaactcgaggtagtgggtgaatgtgtcccaagaatccagcgaaacagggtctcccaggaggcgggactggaagggtccggagaggggccacaggctcctggcctttctaagcacaccaagtgcccagtcgcggacccccgggaccaggatgcgctgacgacccggctggcaggcgggtcctcgtgggcgaggcgagggaggcggcgagagaggagcaatagtttcccaccgctccctctcaggcgcagggtctagagaagcgcgaggggatctagagaagccggaggggaggaagcgcgagtccgcggcccgccccgttgcgtcccacccaccgcgtcccctcccctcccctcccgctgcggaaaagcggccgcgggcggcggcgcccactgtggggcgggcggagcgccgcgggaggcggacgag

SEQ ID NO: 5 - DNA, 760 bp, Homo sapiens
  misc_feature: Activin beta-A gene, regulatory sequence of 5′ upstream region (AN D17357
tgattccaatgtttttctaaaaggtagagtaatcctagccagaggtttcactggctcagtgcatcacccagtagtgtctcagaagccaggaagggctttccattagataatgaattatgaaatgtctcacactggaaaaaccagtcatccgctgagtcatgctgattccaaccaatcccaaacaaagccccagccctcctctgtttcagtggtaccaatgtgtggtgtacaaataagtagtacagtataaaacttcacagtgccaataccatgaagaggagctcagacagctcttaccacatgatacaagagccggctggtggaagagtggggaccagaaaggtaatgctttttaactcttacttctgagctctttacacattcaaagataggaaagctaggaggaattttacaactaattggcatttccaatgtgcattgtgatgtgtacctttttatattattcaggcaggttaatacagcttttaatagtcctagagcatgcaaatagattatatgtttatacaagccactcagcacatatatacaagtacatatgccaaagagaaagctatttttaagagttacattcgcaaacagtaaattcagggaacacacacatactcagatgcagagagaatccaaatattgataagttgcacttatctaaatgctgctattaggactcctgagttgtttagagccattaaacttttggttgtatttcagactttcttgtaaaacttaattgaactgcaaa

SEQ ID NO: 6 - DNA, 1140 bp, Homo sapiens
  misc_feature: NK4 gene, regulatory sequence of 5′ upstream region (AN D17357)
ggagaaacctgaacagaatcccagctccgggccctcagaaggaccccacgctgcccacattgaccttggacctccagcctgcagatcgtgagggaagagacgtcttcgacttagggccccttgtcgtggtacttccttagtttggccccaggaaaccatcccaaaggcaagggcgtggttgtgctcagctgggggaagggggctgggggccgtgaggaggaggtgggaggcccagccaggctggagggtcagaacccgtggagctagaagagcccgtaggggagccccaagattgctgagaccagtgaccttcggccccagatggccttgccttggcccagaagggtcagaaggacctggtcagccaagctcagacagccggcaggatgccttccaccctgcagagggtcctatcttgtcccacaggtagatctacatcaccactagccacccctccaacgtgcacaggcccctgccctcacggcgcccctcttaggtccggcagttcctgcctccttctgatccagaagtttctctggcctctggagccggggcacacctcatgcaaggacagggtccaaattcctttgtccttggatcccacttggctgacgtcaccttcctgtactcagggagtttccccagccagctgtcccgagtctggactttccctctgcccctccccactctcaggctggtggggtggggaaagcagcccattcctgggctcagagactcccaccccagctcagagggagcaggggcccagccagggacggaccctcattcctcccagggaccccagacctctgtctctctcgggtaagtctccatctctgtctgtctctgtctctgtctctgtctctgtctgtttttcacgcactcagcaaggcctcctgccctgagagaggctccgcccactaccccccactttccccataaaaccagctgagtatttgtgccaggaagactgcgtgcagaaggtgactgtctcagtggagctgggtcatctcaggtggggagttggggtccccgaaggtgaggaccctctggggaggagggtgcttctctgagacactttcttttcctcacacctgttcctcgccagcaggccttggctccttgaacttttggccgcca

SEQ ID NO: 7 - DNA, 960 bp, Homo sapiens
  misc_feature: prosaposin gene, 5′-flanking region, exon 1, and partial cds (AN AF057307
ggtttaagcaatttctggcctctgcctcctgaaatagctggaaccaacaggcgagactgccacacgctgactaattttgtatttttagtagagacagggtttcaccatattggccaggctggtcttgaactcctggacctcatgatccgcccgcctcggcctcccaaagtgttaggattacaggagtgagacagcatgcccagcccagacttgcctttgaccaggtgccaccacctgcccccacgtgcccctggccaggactgagccctgtaccctgttacacgactacttattctatgtgaaaccccaagctattctatgtgaaacccgctactacaatgggctaatttttttgtattttttttttgtagagatggggtttcaccacgttgcccaggctagtcttgaaccctccgcccgcctcggcctcccaagtgttgggattacagacgtgtcagccacacgtgcaggccggccaacaatgtggagatttaaaaggtattttacatatataatctctgacctattcaattagtagggcttttcttttatgacctttcccttccctttctccaagttcttcctcactcctcccactatagcccttcctttcgcccctcccattgccccctcctattggcctccccttccggcagcgccctcagaggcgctgagtcagggcgctgttgagctcgggcaggcccggatggggcggggttacgcgcctgcgctctggacggcctttggggcagggcagatttatatctgcgggggatcagctgacgtccgcattgcagactgcggagtcagacggcgctatgtacgccctcttcctcctggccagcctcctgggcacgtcgggtaagccctgggaccctcatcctggggaggaggatttgaccctcgcagcgtccatgtgaccccctcggcctcccaaagtgatgggattacaggcgtgac

SEQ ID NO: 8 - DNA, 3271 bp, Homo sapiens
  misc_feature: 90K (Mac-2 BP) promoter region (AN U91729)
aagcctcccgaatagctgggattaaaggcgcctaccaccatgtttggctaattatttgtatttttttgtagacacggggtttcaccatcttgaccaggctggtcttgaactcctgacctcgtgatctacccacctcagcctcctgaagtgctgggtagtttcttaaaaaggtaaacatatatctaccatatgacccagtaatcctgctcctaggtatttacacaaaataaatacttattttcacacaaagacttgtatccaaatgtttccagcagctttatgcataatagtggaagatggaatgacccaaatgtccatcagtgcaaacatgtattaacagtggtgttctgtccatacagtgggccgccacccagcaaacccaggagccagttactgattgttgagatagcatggatggatctcagaagcactgtggtaagtaaaagaagccacatgcaaaatattaaatactgtatgattccatttagagggaattctagggtccaggagtggtgcctcatgcctgtaatcccagcactttgggaggcagaggcagggcgggatcacctgagttcaggggttcgaggccagcctggccaatgtggagaaaccccttctctactaaaaatacaaaaattagctggccgtggtggtgggcgcacctgtaatcccagctactcgggaggctgaggcaggagaatcacttggacctgagaggcagagattgcagtgagccgagattgttccactgcactccagcctgggcaatggagggagactgtgtcttaaaaaagaagacaaaatagagggaattctaggaaaggcaaccagcagtggcagaagctgagaggtggttgctgggaaggggctgggggaggtggtggctgcagaggggbataagagaattcttaggggtgattgaaacgccctaggtaatgattgttgtcatgataccatcgctacacatttgccaaaactttgcacgtaaattatatgccaagaaagccaatttttaaaaagaaggaaaggatgggtttgaaaccccagttcttcccctaccagctgcacaactttagccgattacgtcgcctcactgagcctctgttttctcatctgtaacagggaatataagagcagctgcttcccatcatggctggaagtattaaatgcattcatttgtggcaaggcttatagtaatgcctggcgaaatccatattagctattatagggagcgttcctcaatttgcggagaggtttggggtagaggcacaaaagatgaccttacaggccagttaaccattctcatctctgaaatgccccgcactttcccttccatgtcttgggagcggcttcctgatgacagcagttctgtccacacgaatctgaggctttcacccagctgtcttctcagagccgagccgctgccccttcccctgcctgtcccctgtcagcgcttccctccaccccatggtcatcgcacaccggaaaggccttgcgagccccaggggagcagatgktyggtgctccgattccacgaggaggcctctgggttttccattttacctgcctggatggcttaggactttcccggactctggggctaaagattcggcacctgagttttaaaacctttcccagcacttcccagagatgccctcccgtcctctgcactcctgtccttccctggccacttgggcagaagtcattagcactgctgagaagggatgatgctggggtttctgtgcactcaggcccttaatccggatgagatttttttaaactccccacagccagttctatttccagctgcacctgcccctggatcttcacaagttcctctggaggggattaggcaaaccgtgcagctgcctaaaacctcacaccttgaaggaaatagtcattgaatgtctgacctctgggctggctgtctcggactctaagctgccagggaaccagggccttccacccagtgggactgcctgggggcttttaaatgcccctgcctgtcccctactcccagagatggtgacttcctgggtctaggcattaggagtttgtaaaactccctgatgattccttctgtccagcccaggctgagaaccactggtcagaggcctgggcacatcccaaggctcatccagaaccatggggtgcaagtgacagaaacaagagcggctgctgattgcctcactgagcagtgaagcccagccttgaccatggattaggccagctggacccaggagctcaggccggaggatgcctgcttccctctgctctgccccaccggccccagcagcctgggcccacatcctctcagtcagaagctggctctcaccggctggctgggctcacagccccaccctgaaaccagcagtgtggcccggggcccccgcaggctcagacagccaggccttgggtggttgaaggccaagagctgggggccctctgggaaccacacagccgggaatgggagggggtgctccccaagggacagttgaggtgccggctttcagtgggaggaaagggaatgggtatgagctggacagagccattatgtcacccagagaggctctgtcccccgccccgctgagggggagacagtaggagagtggccacaggtccagcagtggcgagcacaggctctggggtcaggtgttggagcagggtccagctcctccactggccagctgcatacctggttctcagtgcctccctcccctggggacaggggacagtgccatgcaaccttgtggggcacaggccctctgtgtggtcagcatgccaagagcacagagagggtggatttgcacatgagcagccccctgtgtggtgttcacccagccagcaacgtgctagacccaggaaaagactcggagcgctctgtcagagtccacagccacaccaccaggtgcagactgtctgggcccagagcctctgcttcttcccctcccgtccaccaaacgccagcccctgaccacctggcggcctttccaactgagtgtggctgttagtcctcttgcaggccttgctccagccagactcccaccttgggcctctgccagcctggcactgatagccacaggcagagctgagacaaaagagaggggccctggggagtatcagcagcagccaatcccggaagacatctatgtcaggtggtttctggaaatcgaaagtagactcttttctgaagcatttcctgggatcagcctgaccacgctccatactgggagaggcttctgggtcaaaggaccagtctgcagagggatcc

SEQ ID NO: 9 - DNA, 1403 bp, Homo sapiens
  misc_feature: galectin 3 (LGALS3) gene, exon 1 (AN AF031421)
tccaggccagcagatttgatgtctggtgagggcctgctttctggttcacagagggagccttctggctgtgtcttcacaaggtggaagtggcaaggggactctctccggcctcttttattaaggcaccaatctcattcaccctatgacctaatcacttcccaaggcctccacttcctaatacatcaccgtgagggttaggatttcaacatatgaactttggcgggatataaacattcagactatagcaccctgacagtaaaaatgagataataataacttatctctttcttccaacaaaaagataaggtgaagttaaaaggagggtatatatatatatataatgtgaatttcctgtgtaaaatgtgttaaagagttgtctgattaattgctttataagggaattgctttgagactaggcctattgatctagaataagtagtcaatttgtagtcagttccctagggaatagacattgaaaagatttttggttttgtattctacaaataaagcaacctattaattgaattcctctcagcgaattcttcactcaggtgattctggagagggcgggggacagacgcggccgcagcccaggtcccgggagcgccacggaacctaacggtggcagcggaggtcgcgcccctcagtgcccgcgctctccccgtcgggagcttcctggtcgcccctgcggcggcggctcggggtgtcaggccggcgcggggctcgcccagcctggtccggggagaggactggctgggcaggggcgccgccccgcctcgggagaggcgggccgggcggggctgggagtatttgaggctcggagccaccgccccgccggcgcccgcagcacctcctcgccagcagccgtccggagccagccaacgagcggtgagctgcgcggggcgcgggggacgcggctccggccgggcaggggagagggcgcccgggcgctgcttggggcgcggtccggagagggttcggctccccgggaccgggccggggcgcgcgcggagagccccacagcctgtgctctgccctccaggagcggggcggcgggcagcgatctgggcccggggcagtcgcctttgattatcgagggcgctggcgttcggggaaggttggcagcaccttacgagacccacacacgtccccggggcggcacgggccaccttctgcggagcctcgtgcggcttcgccgccgtcgcacctccgccgcctgcgcctctgcgcgccccagagtaagccccatccggtgacgagccgcagtctggtcaccccagtcccaccaggtcccgctgcgaggggaggcggaggggctcgctcagcaaaccagacggccgctccagtttctctaattggggttggagccccgtcacccttccccagatcacggccgcggggga

SEQ ID NO: 10 - DNA, 859 bp, Homo sapiens
  misc_feature: manganese superoxide dismutase (SOD2) gene (AN S77127)
gatttcactattactagaatcaataataccaaccctaggggtaaaaataaagataaatgtgtgcaaatcctgcctgcagtctcgggcacgtcgtgggtgtccaagaactgttcttaggcagccggtggggacaaagtctgtgtgcctcctgtcctggaataggtcccaaggtcggcttacttgcaaagcaagggtacggcgcaagagtactgaatacgggttggaagggcgctggctctaccctcagctcataggccggctgggcgcggctgaccagcagctaggccccgtcttccctaggaacggccacgggggcctgggagggtatgaatgtctttttgcagtgaggcctctggaccccgcggccccccggcagcgcaaccaaaactcaggggcaggcgccgcagccgcctagtgcagccagatccccgccggcaccctcaggggcggacgggaggcagggccttcgggcgtaccaactccaagggggcaggggccgcctcccttcggccgcgcgccactcaagtacggcagacaggcagcgaggttgccgaggccgaggctagcctgcagcctcctttctcccgtgccctgggcgcggggtgtacggcaagcgcgggcgggcgggacaggcacgcagggcacccccggggttgggcgcggcgggcgcggggcggggcccgcgggggggggggcggggcggcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatg

SEQ ID NO: 11 - DNA, 2877 bp, Homo sapiens
  misc_feature: granulin gene (AN L32588)
cgggaatgcggtaattacgctttgtttttataagtcagattttaatttttattccttaacataacgaaaggtaaaatacataaggcttactaaaagccagataacagtatgcgtatttgcgcgctgatttttgcggtataagatatatactgatatgtatacccgaagtatgtcaaaaagaggtgtgctatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcgcaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggttcaagcgatctcctgcctcggcctcctgagtagggaattacagacctcgttatcgtggcaccttacccttctgatgttaaaaaaaaaaaaaaaaagagcgagagagagagagagaaacatttgtgaagtaggttgttgagtctcagcactattgaccttttgggcaggatacttctttgttgtgggggattgttctgtgtgtcgtgtgatgtttagtgggattgctggcccttacctaccagatcgccagtgtccctccaccctgagttgtgacaacccagattgtctccagacactcctaaatgtccctggcgcaaaattgccgctgctcaagaatcacggactttgacgattagactttgtgatatttgtttcagtctgtttaggttttttttcttctacctgtatttttttctggttctgggtggttgtaattagtaggttattgatcgattcacctaacatttcatgaaagtttcatgtgtgtgtgtgtttcaatagaagcataaactatactccctagtctcaagatagccaggaaggaaaataagcacaaatgtgtcaccagggcacagactagtactaggtcctcagcaggccaggtgtcttatccgctgtctgggtctgctctagctccaggcttagaacccctgccacacgactccacagctcggttggcaccctttccctcctccgacttctgctgcctcgagcttggttagccatccccctgcccctgcctcatcctcagctccagttccttgctcaggctgcagcagtctccatcccctgtgcagacactgccgttcctccacggcccagtatcaggctttccctgggcctctcctctctcctggcccatctcccatcatccatctctgcctggcccaggccctttggcaccaagcaggctgactcttgtcactggctaatctgttctgtggtacattttctctcctcaccctcccatatcaattcctcgaaggcagggcgatctggagactaggaagccacttctctttcgacagcccccaccacagcccagcccgtgccaggcacccagcagctcctgaagcccactggcattgaacatggcattcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgcttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagacc

SEQ ID NO: 12 - DNA, 2040 bp, Homo sapiens
  misc_feature: p66SHC gene, 5′ upstream (2.0 kb) region (AN NT_004524)
tccccggccttgtgctgcttcagtctggccctcgtccctctttaagaggactccatggcaccttcagcctggggtgtggtgggtgccccttcctcctcatcgtcatcagggggccctggggtagagaccgggggcccagtgggggctgactgctcccagaatcgagctagagagaggcggaaggtgtccaggtggccattggagaggtcgaggccagcgggggatgcagcagcggtagagggtggtgggtagtggggtggtgcatcgtccttgcggcgccgtcgggtgcgcacctccaggcagttctggcctttgaggtggcgctgcaggtggtcctccttggcgaaagccttgtggcacaggtggcactcatagggccggtcccctgtgtgcaggtgcatgtggttcttgaggtcgtagctgtgcaggaagcgggctgggcagtgcgggcatgagtaggggcgctctcccgtgtgcttccgcatgtggatcttcagcttgtcgttcctggcacaggcaggggtgaggggcagggaggtgttcaggatgggatccctggctcctcctggtcatcccaccctggccttgtcccatccctgttcctcaccatttgttaccccactattgccccaggagataccctgctagctcacctctagcttaactaggccttgtcattatccactcctctgcttccagcactgccctcctgggcaccgaatcctccatgctccagatccaccccctgctggcttgttttcttctgctctcctcactccagtaacccacccatgccccagtccctccaggaccacctaccaccacctcgggtctcctaacttcttccccatctcctcccaccctgtcccctacccatggcccctgccgggcccttccccccttgctgctcacctggtgaatcgaacaccgcagacctcgcaggcaaagggcttctcgcctgtgtgggtcctcatgtggcgaggcagtttgcctgccccatggatgatcttgtggcagacagggcactcctgaggcatctgggagcggcgtttgcgcaccagcttgtcttggctgtccaggcctggtgccaggttgtcctggtgcagggagcttaggtaggccatcaggtcaggatcgatggcatcctcatctgagcccagctcctctggggacagcgggggcccgccaccctgcgccagcccataggctgggggatataccagctcctcttcttcttcctcaccctcatagggttcgtagctctggggaccctcaggaggggaggcagttcctgtgggagggctgtagctgtcccccggcccactgcccccactgctgcccactctgcccgccacctcctcctcctcataggtcaagggatgggcgggcactgtgggcacctcagggactaggtggtttgctctggcccccttggtttgcaggaaagctttccggggcttgcggctgcggcgggcaacaggccgaggtggcggtggcggaggtggtgggaggggcacctgtggaggactgtcttcaccattgggaactcccagaggccgtggctgtggcaaaggcctccagatactggcgggctcgctcacagtcatcctcgtccgggctgggagcttctagcccactgccctgcagaatctccatgcaagcagcgatgacacacgggatctccagcaggcgggcagcctggagcacagctggcatgttggcgctgctggtggtcagtgtggctgtataggcaaattcaaggagggcgcctagtgcctctggccctacaaagtccagctcacacacaccggcccctgctcccccagtggccgtcccgctacccccggcccccatgacagctccgccaccgccctcagtgaaaagcttcttgaagtagtggctacaggcagctagcacagccctgtgggtgcggtattcaaggccctgcgtccggatggtgaggtcacataggtggcccagctggcgctgctcattgaggcagctcaggagctcactgctgtggtccgggaatggaatccc

SEQ ID NO: 13 - DNA, 20 bp, Homo sapiens
  misc_feature: Analytical sense primer for NK4
agcaccaggccatagaaaga

SEQ ID NO: 14 - DNA, 1774 bp, Homo sapiens
  misc_feature: cathepsin B gene, promoter region (AN AF086639)
ccttatagaggtctgaaatgatttggagtccagagtccatggctgtcaggatatgactagggtgagcaggcagttgggaccaccttgacctccagcctcctggtcctcagttcctcgggtatcccactctgctgggggcttagtgaccatgtttgggctccagagattattttttccttccactcctatccttagtttgttactaaccaggcgggagtacaggcatgtctctgaagacaggctcagggctgtgtgacagctgacgaccaggctgcagggaaccaggtcccatgcagtcctactgccttctttttttttttttttttttttttttttgaggcggagtctcgcttttcgcccaggctggagtgcagtggcacgatctcagctcacgggttcacgccattctcctgcctccgcctcccgagtagctgggactacaggcgcccgccaccacgcccggctaattttttgtatttttactagagacgggtttcaccgtgttagccaggataatcttgatctcctgacctgtgatccgcccgcctcggcctcccaaagtgctgggattacaggcgtgagccactgcacccggctactgccctcttactgtcgccacagcctggataaaatacgattcttctgagccttttttttttttttaatacagagtttcactcttgttgcctaggctggagtgcaatagtgcgatctctggtcaccgcaacctccgctcccgggttcaagcgattctcctgcttcagtctcccgagtagctgggattactgacacgcgccaccacgcccggctagttttgtatttttagtagagacggggtttctccatgttggtcaggatggtctcgaactcccgacctcaggtgactcaccggcctcggcctcccaaaatgctgggattacaggcgtgagccaccgaacccagcccctctgagcctcttgaatacaactggggtcatgtgcctttgcaggtttgtcttaaggattaaagctgtttggggagtgtctggaggagggtgagtcttgagccaacccctgcatctcccttccagggcctcccggtaataaaccccaagtaaatgtgcactttgtccgtcctctcggagcaggtctccgggtactcctgtgccaaaccgatttccgcccccaaggtccttctcctcttagaaatcctgacgcagctcctaggttccttcgcagtgacagccactcttttctatttgtacgtagctgtagtgttttgtgggtacgttctctgaacaacaaagtggcccttctaaaggctgttctgtggggtccacagcctcgccacccccagcctctgcagcggcttctgaatgaatgaaataagcgacggcgccctctccaccaccccacccccgccaactcggcaggcagggatcccaggcgcgggttctggcggaggcggtcccgcgaggcggggggacttttctaggcggggtgggggccttgggaccacctttaggggctttttccccatcccctggccccaattcgcagcgtttcgccacccagggcccgcagggctccaagcccctcttccccagcccgcgcgctcaggcccccgcccgcccccggcggtggccccggaccccgagcggaagggggcggggggtgtgcggggccgggaagcggggagcgcgggcggcggaaggtggcgggagggggtgggggctgggaagcaccgtgcgcgggcggcgggagggcccgggcggggctgcgcggtggtcacgtgg

SEQ ID NO: 15 - DNA, 3804 bp, Homo sapiens
  misc_feature: Promoter DNA for Alzheimer′s disease amyloid A4 precursor protein (PAD, AD-AP, AAP, CVAP)  (AN X12751
ggatcctaacccaatatctgctgtccttataacaagaggagattagggcacagtaagacacagagggaagaccatgtgagaatacagggagaaggtggccatctgcaagccaaggagagaggcctcagaagtaaccaactcagccaacacctcgatttcagacttccagcctcctgaaatgtgaggaaatacatttctggtgtttgatccatccagtctatggtaagttatggcaccctgcagggttcatctggctcagacttaacgattgcttttggtgatatttatagggcacagataacagcctaaacacaagacgacagaaacgcggcccagcagactatgcataaaatagaaatggggtatctggaccaattggagtctgcagtgggatgcggttactaaaacagtcaaatgcaacatgaggctccaggcagagtagtgggcaacatctcccatgttgcagcagtcagagcacacttcgagtactgtaaaaagacacagacaaggcagaacactttagagaatggccaaggtgtggaaggaacgagaaaccatgccattatgcaactgttgaaggaagtgcctgttttaccttgtgaagagaagactctagaggaagaagtagcatgaaaacagctggcaaatttgtaaagatctgaagtgtgcaaaagaattattctgcttggtcactgggcaatacaaggatatctgagtgggagtttaaaggcgggggatgtgagctttaaatgggataagaacattctagtaaccagaaatgcccaaagatagaatgcacagtctggagagccagtgaatatctcacaaatggagacacttgaaactaggatggggatgctgttgtaggaattccagcagacaagtggttgttggttccttccccaactttgtagggttataactagggatgttcctgcgttttctgcttggaggatctgcaagacacctcagggcaggaaatggcattaaatgcagaacagagctagtggctgaaaagcaaaaagccatcaggatctctggagtagtgaaggaaccagagaacatgcaggcaatgtccatcattctgacgcaatcagcagcgataatcatcttcccccaggaacatcttgaccagggaatgtgtcagtgttggtgaatttcaacagtggaaagagaaactgctaaatctaagaactttaatttttataggttatgatctcatctctacaattttgaatttcatgctcaataaaagttccttactctctttttttttttttgagacggagtctcgctctgtcgcccaggctggagtgcagtggcgcgatctcggctcacttcaagctcagctcccgggttcacgccattctcctgcctcagcctcccagtagctgggactacagcgcccgccacgacgcccggctaattttttgtatttttagtagagacggggtttcaccgtgttagccaggatggtgttgatctcctgacctcgtgatccgcccgcctcagcctcccaaagaaaagtccctcactcttaaagttgcctcctccttcccagggctggcttcatgggcatgcaaccctggagagtctcacaggccctgcggtgggaggagccccatgcttggtttaacgctctgccattgccatcttaaaattcttaatttaattttttttctttttttttgaggtggagtctcgctctgtcgcccaggctggagtgcaatggcacaatcttggctcactgcaacctccgcctcccaggttcaagcgattctcctgcctcagcctctggagtagctgggattacaggcaggagtaaccacgctcggctaatttttgcatttttagtagagatgggggtttcaccatgttggccaggctggtctagaactcctgacctcaggtgatctcccaccctgggcctcctaaagtgctgggattacaggcatgagccaccaggcccggccttaaaattcttaataatgtaacaaagggtctcacgtttgcattttgcagtggactctgcaagattgtagcttggaccacgttctcttgcattcagataccttctttttgccttatttgctcatgcagacccggaacaaatacggaattgcggtggtaaatgtggtgcagaaagtgaacaactgggtttgtcctgtcactttaggcttttccctgtgtcccagcttcatgtcacttacttgctattagatttgggagttcattagcttcattttcctgatgtataaataggaataatagtaacagcctctttggcttttgtaggaagtaaatgacatgaagcgtataaacaaatactgcatgacaataaatatttgtccttatttgttgaggacatccaaaggacattcaggggcaaaagtaatccaagagtcaagactgaatgcctagtgcggaaaaagacacacaagacaacatttaggggagctggtacagaaatgacttcccagaagaagtctgtaccccgctgcctgagccatccttcccgggcctcggcacccttgtcagcgcaatgagcaagggagagaaggcagcagtgcagcctcagaagggccagcgcactccctggcttcagtccttcgctccaagccctgtgtggagtgggctgtggcttggtaactaaacgctacttcaggtcaagagcaggggatatatctgggcagttctagagcattctaaactatctggacactaactggacagtggacggtttgtgtttaatccaggagaaagtggcatggcagaaggttcatttctataattcaggacagacacaatgaagaacaagggcagcgtttgaggtcagaagtcctcatttacggggtcgaatacgaatgatctctcctaatttttccttcttccccaactcagatggatgttacatccctgcttaacaacaaaaaaagaccccccgccccgcaaaatccacactgaccaccccctttaacaaaacaaaaccaaaaacaaacaaaaatataagaaagaaacaaaacccaagcccagaaccctgctttcaagaagaagtaaatgggttggccgcttctttgccagggcctgcgccttgctcctttggttcgttctaaagatagaaattccaggttgctcgtgcctgcttttgacgttgggggttaaaaaatgaggttttgctgtctcaacaagcaaagaaaatcctatttcctttaagcttcactcgttctcattctcttccagaaacgcctgccccacctctccaaaccgagagaaaaaacgaaatgcggataaaaacgcaccctagcagcagtcctttatacgacacccccgggaggcctgcggggtcggatgattcaagctcacggggacgagcaggagcgctctcgacttttctagagcctcagcgtcctaggactcacctttccctgatcctgcaccgtccctctcctggccccagactctccctcccactgttcacgaagcccaggtggccgtcggccggggagcggagggggcgcgtggggtgcaggcggcgccaaggcgctgcacctgtgggcgcggggcgagggcccctcccggcgcgagcgggcgcagttccccggcggcgccgctaggggtctctctcgggtgccgagcggggtgggccggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcc

SEQ ID NO: 16 - DNA, 1741 bp, Homo sapiens
  misc_feature: Tissue transglutaminase gene, promoter region and 5′ UTR (AN U13920
aagctttcaccagctggagggagcagtttctgcaacaatctctataaaatggggcaattacgggtcagctgggcccaacactctttgtgggtttgttcactgagactccagccagagcccgtttgacccagggagaaatatccactgaagcaacacgggttgttttccctgagccatatgtcacctaggaatggagacgggggctacttctatcttccaaattcatcaatagatgtagagcttgttccggaatgtacagcttgttctggaatgtagagcttgctccggaatgtagagcttgttttggaaaaagtgccggggaagccccgtgggcctctgtctctccgggaacccttcccgctcacggctcacagtggatccggaagcacaggagaccaagagaccagagataccaggatgagagataggacccctggttgccaggttcgagaagtcctaggctgagtccctggaaagttagtcttgctcctttctggcacacagtggggcctcaagaaagctcagtggatggatggattgagggagggagggaagagaaggccgagggagggagggaagagaaggccgagggagggatggatgaagggatgagtggatggataggtgggggggtaggtgatgcatgggtgagtggatggatgggtagatggatggctgattagatggatggctgattagacagatacaaggatgggtgagatcggaggattatctgggtttgctacaggaagggacatgggtgtgtctgtttttggaggtgtgtctgcatgtctgtacctgagtccatgcctgcatgtgtgtctacctctgagtagccacatctttgtgtgtctacctctgagtagccacatctttgtgtgtctgtgggtgccctctctgattttgggtccacatctgacagaggcattggtgtctaggaggtctgtgtgtgtgccaggtgcctctggacacctgctcatctgtgtccacagatgtgtgtggctcgcggacaaggctacctggctgtgtcagggtgtatctatgtcctggtgtgtgtctgccatacgaatctgaatttgtatccatgtcactgtgtctgcgtggccagccgtgtttggtgaatctgtgggagtgtatctgtgtatgtgtgtgtatcaccacagccctgtcttggtgtgtctgcgtctgctctccgtgtatgtatatctgagtatgtgtgtgagtgtgtgcgtgtgtgtgtgtgtgtgtgtgtgtgtgtgtgtgttgggggtggggaggtgttcttgatcccagatctgacctaagagtccacatctgtgtgtccaggtgcaccccggttccgttgtgtgtttctgtgagggtgctgcgtgtatctgtatctgagtgtgtgtgtccaggtgtctgttctccaaggtctgagactgtgggtccaggtgtgtctgtttcctgggctagttgtgtgtccctgtccgctcccccagggggcgccctcgtccgaccgccgtccctccctcgggctccggtcccctgggtgagccccagcgctggcggcgtgggcccgggactggacaatgggtgtcctcccaggtcgccgccttcccgcggggccccgcccccggcccgccccaaagcgggctataagttagcgccgctctccgcctcggcagtgccagccgccagtggtcgcacttggagggtctcgccgccagtggaaggagccaccgcccccgcccgaccatgg

SEQ ID NO: 17 - DNA, 1440 bp, Homo sapiens
  misc_feature: Clusterin (APOJ, SGP-2, SP-40, TRPM-2) protein gene (AN M63376)
gacctgcaggtcaacggatccattcccgattcctcatcgtccagatggaagaaactgaggcccaagggcaaagtgattagtccgaggtcacccagtgtctaggggcacacctaggactgtaatcagactttcatggacctggtctgggttctcccacttagtcatgggccttgaagattccccgaggctgcctcctgaaaaggactggggtctagtggcccctggacgttgggcaagcaagggactgggcctccatgttgtgcctccatagtcctgatcctgaactggaaaactcagcccctgaccacgcagctctcctttaagcccctttgtttcacatggttttcaaagtctgccacccacagtggggctgcctgtacccgccctgtccacccattgccccagctgtcagccccttgacttctctcctggggcttaaacatccctggctccaaaatgggcagctcactttcttccccaagaagtagctgcacctccagggttcctagatttgcccctccttgccagggggaggggtggctgcgacaggagattctccctgctctcagcagaaggaactccagcagttggagaccagcaaacccctctggacacagatctgatttcctaactgggaaggctcagggcaaaataaaaattcaggtccactggttcaaaaactatgaagaatttcaagaccgtcacagtagcccattaaaccaaacgtggatctgcaagggtcccacagccatgaagcccaccctgcttggttgggttccaaaaagatggggacagtgattgcttaagctctgtggatcaaggaccccggagaggccttctggctctccacatatctgctctgatcactcctaaacacaattctgtttcctccaggcctggcgggtcagtccagggacccccatcagtgtgatgtttccaggagtaggcgtttcaatacttcctgtgctctcttctccagcacaaggcccctctccatcccaccctcattatgtctgactctttactatttaaatgggtcaagagaagtggcgcttgtgtaatgtgaaggttaaggtcagtagggccagggaactgtgagattgtgtcttggactgggacagacagccgggctaaccgcgtgagagggctcccagatggcacgcgagttcaggctcttccctactggaagcgccagcgccgcacctcagggtctctcctggagccagcacagctattcgtggtgatgatgcgcccccccgcgccccagcccggtgctgcaccggcccccacctcccggcttccagaaagctccccttgctttccgcggcattctttgggcgtgagtcatgcaggtttgcagccagccccaaaggtgtgtgcgcgaacggagcgctataaatacggcgcctcccagtgcccacaacgcggcgtcgccaggag

SEQ ID NO: 18 - DNA, 2000 bp, Homo sapiens
  misc_feature: Prostacyclin stimulating factor (PSF, IGFBP-7, mac25) gene, 5′ up stream (2.0 kb) region (AN AC022483
gatgcccagtctcttctcttgggtggcaggtgctgggacctgcaatgtgtattcttggattttagagcttatggtggagggtggagcagtgagtagggcatggatcctcgactcatgtctacagcaaagtggggcagagcaaccatatttagcaccaaatacaagcaggagtggatccaggctttgaagggcctggggcttaaacaatttggggattctctttaagaaaaaaagatataaaattaagtatgtataaataatgaatatttattacttaataaatatttactattaataaataatacacaaatatttattatttattcaaaataaatattcatttggaataagtgaagtaataatagtaatttttcaaatgtagaaatgctggcagatattacaaacatcaacacatttttaaaaactaatatttttattcattatctgtccaacacacctctataaattttttggtaatttttatatgattaataattcttaaaaatatataaaactaatctgaaattaaaatatacagaagagcacttgttttttttttttttttgagatggagttttgctgttgttgcccaggctgcagtgcaatggcgtgatcttggcccactgcaacctccgcctcctgggttccgacaattctcctgcctcggcctcctgagtagctgggatgtcaggcacccaccaccatgtctggctagtttttgtatttttagtagaaacggggtttcactgtgttgcccagcctggtcttgaactcctgacctcaagtgatctgcccaccacagcctccccggctaatttttgtattttctagtagagatggggtttcaccatgttggccaggctggtcttgaactcctgaccttaagtgatctgcccacttcagcctcccaaagtgctgggattagaggcgtgatccactgggcccagcctcagaagagcaattttaaattgtacttgtgttgaactatattataattattaatctaattataattatgtaatcaaattactattacttacattgatttattaatgaatatgtataggagttttgacataagaaaactcctcaggccattttgccatttctgtgtcaatgttgtgtgccttttcgtcaatgaacagacctcgtcagcccaagagcatcagatgtgctaagaggtgatgtgatctgattggatgcataaaatgtgggacttcccacacagatgggcttgctgttggtgatactgctacagtttatgccctacaaatccaggaattgtgaccaatcctattttgtgacattcccatcaaaatatatatgtgtattatgtgttaataattgtgtacactctcctatcaagtatatttctgatagtagcaaacttttgttttaaccaggtatcaatgagaactgaatcttccatttaaaactgtatacctctgatgattggaagcattttctgaagactagcttttggctccagacatttcaaactgtattttccctccattacttacatatatttctggtggtgggcaccgttggacacgttcataccacaatttgacccttggctctgcactttggtgttatgacactagatgagttggctcaatgggattaggaatatttctggaagtcattcctacaccaagagggctggtaatagcctaactaaacataaaagcgactgcaaaccacataaatatatgccactcaatccaaacttcatgtatccccaactcaagttgtccttagtcagatgccaaaaatgcctgccaccaactcatcactactgaatagaacgctgatggtgagaaggtcagagaggaaagacagtgatcttaaacaaatgctgttaaaatacttttattttccaaattgtataaaatcacatggctataggaacatattgttagggctgctcaaggggtgttgcatggggcacatgaatgtaaaacttgatctccaatagcttcccttagcaatacata

SEQ ID NO: 19 - DNA, 1127 bp, Homo sapiens
  misc_feature: Vascular endothelial growth factor C gene, partial cds and 5′ ups tream region (AN AF020393
gttcttggatcatcaggcaactttcaactacacagaccaagggagagaggggacccctccgaggtcccatagggttctctgacatagtgatgacctttttccaaactttgagcagggcgctgggggccaggcgtgcgggagggaggacaagaactcgggagtggccgaggataaagcgggggctccctccaccccacggtgcccagtttctccccgctgcacgtggtccagggtggtcgcatcacctctaaagccggtcccgccaaccgccagccccgggactgaacttgcccctccggccgcccgctccccgcaggggacaggggcggggagggagagatccagaggggggctgggggaggtggggccgccggggaggaggcgagggaaacggggagctccagggagacggcttccgagggagagtgagaggggagggcagcccgggctcggcacgctccctccctcggccgctttctctcacataagcgcaggcagagggcgcgtcagtcatgccctgcccctgcgcccgccgccgccgccgccgccgctcagcccggcgcgctctggaggatcctgcgccgcggcgctcccgggccccgccgccgccagccgccccggcggccctcctcccgcccccggcaccgccgccagcgcccccgccgcagcgcccgcggcccggctcctctcacttcggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatg

SEQ ID NO: 20 - DNA, 800 bp, Homo sapiens
  misc_feature: TIMP-1 (tissue inhibitor of metalloproteinases-1) gene, promoter region (AN D26513
agaaccggtacccatctcagagatttgttgtgagctttgagtgagataaaatatgctgagtgcctggatatcagtaggtgctgtataatatgccggctatttgcctgtgttatttgagaccctggctttgctcctggccacctgagttccagtctcagttctgccatgtattgactctgtgatcctgggtaagtcacttaaccactccgtgcctcagtttccccgattttgtattcctcccctttcacctgccttatctccctccactgctgctacttaatttgtttcctctctgccacccctcaccagcatgtcagacatacaaaatcaaggcatttttgtgtgcttggcacacagtagatgcacaataaatgttgaagggctgaactaatttgggtttgagtcatagggagacttgggggagtgtgggtgattggatagattctggagactttaggggactgggccgggggaaatgcggcctctaagctctcgctgaggcggcttggaaggaatagtgactgacgtggaggtgggggaggtggctggcccggtcgaggcccagggagagggagaggaggcgggtgggagaggaggagggtgtatctcctttcgtcggcccgccccttggcttctgcactgatggtgggtggatgagtaatgcatccaggaagcctggaggcctgtggtttccgcacccgctgccacccccgcccctagcgtggacatttatcctctagcgctcaggccctgccgccatcgccgcagatccagcgcccagagagacaccagaggtacag

SEQ ID NO: 21 - DNA, 27 bp, Homo sapiens
  misc_feature: Sense PCR primer for CC3 promoter (spec Table IIIa)
gctaagaggatattgacattagacagg

SEQ ID NO: 22 - DNA, 20 bp, Homo sapiens
  misc_feature: Antisense PCR primer for CC3 promoter (spec Table IIIa)
agggggaggtgggttagtag

SEQ ID NO: 23 - DNA, 22 bp, Homo sapiens
  misc_feature: Sense primer for NK4 promoter (Table IIIa)
tggagctagaagagcccgtagg

SEQ ID NO: 24 - DNA, 21 bp, Homo sapiens
  misc_feature: Antisense primer for NK4 promoter (Table IIIa)
gccaaaagttcaaggagccaa

SEQ ID NO: 25 - DNA, 23 bp, Homo sapiens
  misc_feature: Sense primer for SAA promoter (Table IIIa)
cagagttgctgctatgtccacca

SEQ ID NO: 26 - DNA, 22 bp, Homo sapiens
  misc_feature: Antisense primer for SAA promoter (Table IIIa)
cactccttgtgtgctcctcacc

SEQ ID NO: 27 - DNA, 20 bp, Homo sapiens
  misc_feature: Sense primer for beta-APP promoter (Table IIIa)
ttgctcctttggttcgttct

SEQ ID NO: 28 - DNA, 18 bp, Homo sapiens
  misc_feature: Antisense primer for beta-APP promoter (Table IIIa)
gctgccgaggaaactgac

SEQ ID NO: 29 - DNA, 28 bp, Homo sapiens
  misc_feature: Sense primer for t-TGase promoter (Table IIIa)
cccagggagaaatatccactgaagcaac

SEQ ID NO: 30 - DNA, 28 bp, Homo sapiens
  misc_feature: Antisense primer for t-TGase promoter (Table IIIa)
tcgggcgggggcggtggctccttccact

SEQ ID NO: 31 - DNA, 25 bp, Homo sapiens
  misc_feature: Sense primer for CTGF promoter
gcctcttcagctacctacttcctaa

SEQ ID NO: 32 - DNA, 18 bp, Homo sapiens
  misc_feature: Antisense primer for CTGF promoter
cgaggaggaccacgaagg

SEQ ID NO: 33 - DNA, 21 bp, Homo sapiens
  misc_feature: Sense primer for integrin B3 promoter
gattggtcttgccctcaacag

SEQ ID NO: 34 - DNA, 18 bp, Homo sapiens
  misc_feature: Antisense primer for integrin B3 promoter
ccagcacagtcgcccaga

SEQ ID NO: 35 - DNA, 24 bp, Homo sapiens
  misc_feature: Sense primer for activin promoter
tgattccaatgtttttctaaaagg

SEQ ID NO: 36 - DNA, 23 bp, Homo sapiens
  misc_feature: Antisense primer for activin promoter
gaatgtctaaagagctcagaagt

SEQ ID NO: 37 - DNA, 23 bp, Homo sapiens
  misc_feature: Sense primer for prosaposin promoter
ggtttaagcaatttctggcctct

SEQ ID NO: 38 - DNA, 25 bp, Homo sapiens
  misc_feature: Antisense primer for prosaposin promoter
cgtctgactctccgcagtctgcaat

SEQ ID NO: 39 - DNA, 25 bp, Homo sapiens
  misc_feature: Sense primer for Mac2-BP promoter
gtaaaactccctgatgattccttct

SEQ ID NO: 40 - DNA, 22 bp, Homo sapiens
  misc_feature: Antisense primer for Mac2-BP promoter
ctctgcagactggtcctttgac

SEQ ID NO: 41 - DNA, 22 bp, Homo sapiens
  misc_feature: Sense primer for GAL-3 promoter
tgtcttcacaaggtggaagtgg

SEQ ID NO: 42 - DNA, 18 bp, Homo sapiens
  misc_feature: Antisense primer for GAL-3 promoter
ctggagggcagagcacag

SEQ ID NO: 43 - DNA, 25 bp, Homo sapiens
  misc_feature: Sense primer for Mn-SOD promoter
taccaaccctaggggtaaaaataaa

SEQ ID NO: 44 - DNA, 22 bp, Homo sapiens
  misc_feature: Antisense primer for Mn-SOD promoter
atgctgctagtgctggtgctac

SEQ ID NO: 45 - DNA, 25 bp, Homo sapiens
  misc_feature: Sense primer for granulin promoter
gagactaggaagccacttctctttc

SEQ ID NO: 46 - DNA, 25 bp, Homo sapiens
  misc_feature: Antisense primer for granulin promoter
ctggaatgctgtgttcttttctact

SEQ ID NO: 47 - DNA, 18 bp, Homo sapiens
  misc_feature: Sense primer for p66shc promoter
gtggcagacagggcactc

SEQ ID NO: 48 - DNA, 19 bp, Homo sapiens
  misc_feature: Antisense primer for p66shc promoter
ctcctgagctgcctcaatg

SEQ ID NO: 49 - DNA, 20 bp, Homo sapiens
  misc_feature: Analytical antisense primer for NK4
ggtgtcagctcctccttgtc

SEQ ID NO: 50 - DNA, 20 bp, Homo sapiens
  misc_feature: Analytical sense primer for t-TGase
actacaactcggcccatgac

SEQ ID NO: 51 - DNA, 20 bp, Homo sapiens
  misc_feature: Sense primer for cathepsin B promoter
ctcccgagtagctgggatta

SEQ ID NO: 52 - DNA, 18 bp, Homo sapiens
  misc_feature: Antisense primer for cathepsin B promoter
ccacgtgaccaccgcgca

SEQ ID NO: 53 - DNA, 20 bp, Homo sapiens
  misc_feature: Sense primer for clusterin promoter
agccccttgacttctctcct

SEQ ID NO: 54 - DNA, 19 bp, Homo sapiens
  misc_feature: Antisense primer for clusterin promoter
ctcctggcgacgccgcgtt

SEQ ID NO: 55 - DNA, 24 bp, Homo sapiens
  misc_feature: Sense primer for PSF promoter
aaagtgctgggattagaggcgtga

SEQ ID NO: 56 - DNA, 28 bp, Homo sapiens
  misc_feature: Antisense primer for PSF promoter
tatgtattgctaagggaagctattggag

SEQ ID NO: 57 - DNA, 23 bp, Homo sapiens
  misc_feature: Sense primer for VEGF-C promoter
gttcttggatcatcaggcaactt

SEQ ID NO: 58 - DNA, 19 bp, Homo sapiens
  misc_feature: Antisense primer for VEGF-C promoter
gtggaaggaccgggggtgg

SEQ ID NO: 59 - DNA, 21 bp, Homo sapiens
  misc_feature: Sense primer for TIMP-1 promoter
agaaccggtacccatctcaga

SEQ ID NO: 60 - DNA, 21 bp, Homo sapiens
  misc_feature: Antisense primer for TIMP-1 promoter
ctgtacctctggtgtctctct

SEQ ID NO: 61 - DNA, 20 bp, Homo sapiens
  misc_feature: Analytical antisense primer for t-TGase
gccagtttgttcaggtggtt

SEQ ID NO: 62 - DNA, 20 bp, Homo sapiens
  misc_feature: Analytical sense primer for APP
ctcgttcctgacaagtgcaa

SEQ ID NO: 63 - DNA, 20 bp, Homo sapiens
  misc_feature: Analytical antisense primer for APP
tgttcagagcacacctctcg

SEQ ID NO: 64 - DNA, 20 bp, Homo sapiens
  misc_feature: Analytical sense primer for p66(shc)
gagggtgtggttcggactaa

SEQ ID NO: 65 - DNA, 20 bp, Homo sapiens
  misc_feature: Analytical antisense primer for p66(shc)
gcccagaggtgtgatttgtt

SEQ ID NO: 66 - DNA, 20 bp, Homo sapiens
  misc_feature: Analytical sense primer for CTFG
ggagagtccttccagagcag

SEQ ID NO: 67 - DNA, 20 bp, Homo sapiens
  misc_feature: Analytical antisense primer for CTGF
atgtcttcatgctggtgcag

SEQ ID NO: 68 - DNA, 20 bp, Homo sapiens
  misc_feature: Analytical sense primer for MAC2-BP
accatgagtgtggatgctga

SEQ ID NO: 69 - DNA, 20 bp, Homo sapiens
  misc_feature: Analytical antisense primer for MAC2-BP
acagggacaggttgaactgc

SEQ ID NO: 70 - DNA, 20 bp, Homo sapiens
  misc_feature: Analytical sense primer for granulin
accacggacctcctcactaa

SEQ ID NO: 71 - DNA, 20 bp, Homo sapiens
  misc_feature: Analytical antisense primer for granulin
acactgcccctcagctacac

SEQ ID NO: 72 - DNA, 20 bp, Homo sapiens
  misc_feature: Analytical sense primer for prosaposin
ccagagctggacatgactga

SEQ ID NO: 73 - DNA, 20 bp, Homo sapiens
  misc_feature: Analytical antisense primer for prosaposin
gtcacctccttcaccaggaa

SEQ ID NO: 74 - DNA, 20 bp, Homo sapiens
  misc_feature: Analytical sense primer for SOD2
caaattgctgcttgtccaaa

SEQ ID NO: 75 - DNA, 20 bp, Homo sapiens
  misc_feature: Analytical antisense primer for SOD2
catccctacaagtccccaaa

SEQ ID NO: 76 - DNA, 23 bp, Homo sapiens
  misc_feature: Analytical sense primer for beta-actin
gggaaatcgtgcgtgacattaag

SEQ ID NO: 77 - DNA, 22 bp, Homo sapiens
  misc_feature: Analytical antisense primer for beta-actin
tgtgttggcgtacaggtctttg